SEQUENCE LISTING (69 sequences)

SEQ ID NO: 1 - protein, 140 aa, Mus musculus
MVLLRSLFILQVLVRMGLTYNFSNCNFTSITKIYCNIIFHDLTGDLKGAKFEQIEDCESKPACLLKIEYYTLNPIPGCPSLPDKTFARRTREALNDHCPGYPETERNDGTQEMAQEVQNICLNQTSQILRLWYSFMQSPE

SEQ ID NO: 2 - DNA, 423 bp, Mus musculus
atggttcttctcaggagcctcttcatcctgcaagtactagtacggatggggctaacttacaacttttctaactgcaacttcacgtcaattacgaaaatatattgtaacataatttttcatgacctgactggagatttgaaaggggctaagttcgagcaaatcgaggactgtgagagcaagccagcttgtctcctgaaaatcgagtactatactctcaatcctatccctggctgcccttcactccccgacaaaacatttgcccggagaacaagagaagccctcaatgaccactgcccaggctaccctgaaactgagagaaatgacggtactcaggaaatggcacaagaagtccaaaacatctgcctgaatcaaacctcacaaattctaagattgtggtattccttcatgcaatctccagaataa

SEQ ID NO: 3 - protein, 159 aa, Homo sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 4 - DNA, 480 bp, Homo sapiens
atgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 5 - protein, 150 aa, Artificial Sequence
  Hybrid Tslp protein (mouse signal P + human isoform 1 ecto + C-ter)
MVLLRSLFILQVLVRMGLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 6 - DNA, 453 bp, Artificial Sequence
  Mouse/Human Tslp mRNA (CDS)  (mouse signal P + human isoform 1 ecto + C-ter)
atggttcttctcaggagcctcttcatcctgcaagtactagtacggatggggctaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaa

SEQ ID NO: 7 - protein, 136 aa, Rattus norvegicus
MVLFRYLFILQVVRLALTYNFSNCNFEMILRIYHATIFRDLLKDLNGILFDQIEDCDSRTACLLKIDHHTFNPVPGCPSLPEKAFALKTKAALINYCPGYSETERNGTLEMTREIRNICLNQTSQILGLWLSCIQS

SEQ ID NO: 8 - DNA, 411 bp, Rattus norvegicus
atggttcttttcaggtacctctttatcctgcaagtggtacggctggcactaacttacaacttttctaactgtaacttcgagatgattttgagaatatatcatgcaacaatttttcgtgacctgcttaaagatttgaatgggatcttgttcgaccaaatcgaggactgtgacagcaggacagcttgtctcctgaaaatcgaccaccataccttcaatcctgtccctggctgcccgtcactccccgagaaagcgttcgctttgaaaacgaaagcggccctcattaactactgcccaggctactctgaaactgagagaaatggtactctggaaatgacacgagaaatcagaaacatctgcctgaatcaaacctcacaaattctaggattgtggctttcctgcattcaatcttga

SEQ ID NO: 9 - DNA, 22 bp, Artificial Sequence
  synthetic oligonucleotide
cagatgcggacatccaaaggat

SEQ ID NO: 10 - DNA, 26 bp, Artificial Sequence
  synthetic oligonucleotide
tactcacaagcatagtgctatgtgca

SEQ ID NO: 11 - DNA, 20 bp, Artificial Sequence
  synthetic oligonucleotide
cccttccctcaagccataac

SEQ ID NO: 12 - DNA, 23 bp, Artificial Sequence
  synthetic oligonucleotide
gcccagtgtactactcaaaggta

SEQ ID NO: 13 - DNA, 25 bp, Artificial Sequence
  synthetic oligonucleotide
tactgcaatcctctttaaaataagc

SEQ ID NO: 14 - DNA, 24 bp, Artificial Sequence
  synthetic oligonucleotide
cccattgtctagatgtgtcacaga

SEQ ID NO: 15 - DNA, 25 bp, Artificial Sequence
  synthetic oligonucleotide
ggctgacaacagatatggatattgg

SEQ ID NO: 16 - DNA, 25 bp, Artificial Sequence
  synthetic oligonucleotide
actgcttggtacagaatgggaatcc

SEQ ID NO: 17 - DNA, 21 bp, Artificial Sequence
  synthetic oligonucleotide
cacggcttcatgtcttagctg

SEQ ID NO: 18 - DNA, 21 bp, Artificial Sequence
  synthetic oligonucleotide
gtgctgagagacagggcattc

SEQ ID NO: 19 - DNA, 26 bp, Artificial Sequence
  synthetic oligonucleotide
tggagaagcacatgcaatcataccgt

SEQ ID NO: 20 - DNA, 21 bp, Artificial Sequence
  synthetic oligonucleotide
ggctgagtggcactatgtttc

SEQ ID NO: 21 - protein, 370 aa, Mus musculus
MAWALAVILLPRLLAAAAAAAAVTSRGDVTVVCHDLETVEVTWGSGPDHHGANLSLEFRYGTGALQPCPRYFLSGAGVTSGCILPAARAGLLELALRDGGGAMVFKARQRASAWLKPRPPWNVTLLWTPDGDVTVSWPAHSYLGLDYEVQHRESNDDEDAWQTTSGPCCDLTVGGLDPVRCYDFRVRASPRAAHYGLEAQPSEWTAVTRLSGAASAGDPCAAHLPPLASCTASPAPSPALAPPLLPLGCGLAALLTLSLLLAALRLRRVKDALLPCVPDPSGSFPGLFEKHHGNFQAWIADAQATAPPARTEEEDDLIHTKAKRVEPEDGTSLCTVPRPPSFEPRGPGGGAMVSVGGATFMVGDSGYMTL

SEQ ID NO: 22 - DNA, 1113 bp, Mus musculus
atggcatgggcactcgcggtcatcctcctgcctcggctccttgcggcggcagcggcggcggcggcggtgacgtcacggggtgatgtcacagtcgtctgccatgacctggagacggtggaggtcacgtggggctcgggccccgaccaccacggcgccaacttgagcctggagttccgttatggcactggcgccctgcaaccctgcccgcgatatttcctgtccggcgctggtgtcacttccgggtgcatcctccccgcggcgagggcggggctgctggagctggcactgcgcgacggaggcggggccatggtgtttaaggctaggcagcgcgcgtccgcctggctgaagccccgcccaccttggaatgtgacgctgctctggacaccagacggggacgtgactgtctcctggcctgcccactcctacctgggcctggactacgaggtgcagcaccgggagagcaatgacgatgaggacgcctggcagacgacctcagggccctgctgtgacttgacagtgggcgggctcgaccccgtacgctgctatgacttccgggttcgggcgtcgccccgggccgcgcactatggcctggaggcgcagcctagcgagtggacagcggtgacaaggctttccggggcagcatccgcgggtgacccctgcgccgcccaccttccccccctagcctcctgtaccgcaagccccgccccatccccggccctggccccgcccctcctgcccctgggctgcggcctagcagcgctgctgacactgtccctgctcctggccgccctgaggcttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtga

SEQ ID NO: 23 - protein, 371 aa, Homo sapiens
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 24 - DNA, 1116 bp, Homo sapiens
atggggcggctggttctgctgtggggagctgccgtctttctgctgggaggctggatggctttggggcaaggaggagcagcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaatttattttaatttccagcctggccatccttctgatggtgtctctcctccttctgtctttatggaaattatggagagtgaagaagtttctcattcccagcgtgccagacccgaaatccatcttccccgggctctttgagatacaccaagggaacttccaggagtggatcacagacacccagaacgtggcccacctccacaagatggcaggtgcagagcaagaaagtggccccgaggagcccctggtagtccagttggccaagactgaagccgagtctcccaggatgctggacccacagaccgaggagaaagaggcctctgggggatccctccagcttccccaccagcccctccaaggcggtgatgtggtcacaatcgggggcttcacctttgtgatgaatgaccgctcctacgtggcgttgtga

SEQ ID NO: 25 - protein, 358 aa, Artificial Sequence
  Hybrid Tslpr protein (mouse signal P + most human ecto +mouse TM+mouse C-ter)
MAWALAVILLPRLLAAAAAAAAVTSRAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKLLPLGCGLAALLTLSLLLAALRLRRVKDALLPCVPDPSGSFPGLFEKHHGNFQAWIADAQATAPPARTEEEDDLIHTKAKRVEPEDGTSLCTVPRPPSFEPRGPGGGAMVSVGGATFMVGDSGYMTL

SEQ ID NO: 26 - DNA, 1077 bp, Artificial Sequence
  Hybrid Tslpr mRNA
atggcatgggcactcgcggtcatcctcctgcctcggctccttgcggcggcagcggcggcggcggcggtgacgtcacgggcagaaggagtacagattcagatcatctacttcaatttagaaaccgtgcaggtgacatggaatgccagcaaatactccaggaccaacctgactttccactacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattacctgaaacccagttccccgaagcacgtgagattttcgtggcatcaggatgcagtgacggtgacgtgttctgacctgtcctacggggatctcctctatgaggttcagtaccggagccccttcgacaccgagtggcagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcgggatgcctgtgcagagacaccaacgcctcccaaaccaaagctgtccaaactcctgcccctgggctgcggcctagcagcgctgctgacactgtccctgctcctggccgccctgaggcttcgcagggtgaaagatgcgctgctgccctgcgtccctgaccccagcggctccttccctggactctttgagaagcatcacgggaacttccaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtga

SEQ ID NO: 27 - protein, 360 aa, Rattus norvegicus
MRAVTWAIVAMLLPRVLGAIPTRTPRTGGVGDTLSVAIVCHDLESVEVTWGPGSAHHGLSANLSLEFRYGNQVPQPCPHYFLLDSVRAGCVLPMGKGLLEVVLREGGGAKLFSRKKKASAWLRPRPPWNVTLSWVGDTVAVSCPSHSYPGLEYEVQHRDDFDPEWQSTSAPFCNLTVGGLDPGRCYDFRVRATPQDFYYGPEARPSKWTGVASLQGVGPTGSCTGPTLPRTPGTPTPPLALACGLAVALLTLVLLLALLRMRRVKEALLPGVPDPRGSFPGLFEKHHGNFQAWIADSQAAVPTVPEQDKDDDVIRPQTKGVETQEDDDVIAPGSPCLGGGALMSVGGASFLMGDSGYTTL

SEQ ID NO: 28 - DNA, 1209 bp, Rattus norvegicus
accctagaactcgcgaccccgccgaggccccgcccctatcatgcgagctgtgacctgggccatcgtggccatgctcctgccgcgggtcttgggggcgattccgacgaggacgccacggacagggggcgtcggtgacaccctctctgttgccattgtttgccatgacctggagagcgtggaagtcacgtggggcccgggctctgcccaccatgggctgtcagccaatctcagcctggagttccggtatggaaaccaggtcccccagccctgcccacactactttctgttggacagcgtcagagcaggctgtgtcctccccatggggaaggggcttctggaggtggtgctgcgtgagggaggcggagccaagctgttctcccggaagaagaaggcatcggcctggctgaggccccgccctccatggaacgtcaccctgagctgggtaggggacactgttgctgtttcctgcccctcccactcttaccctgggctggaatatgaggtgcagcacagagatgacttcgaccctgaatggcagtcgacctctgcaccattctgcaacctgacagtgggcgggctggaccctgggcgctgctacgacttccgggtgcgggcgacgccccaggatttctactatggccccgaggcgcggcccagcaagtggacaggcgtggccagcctgcagggagtgggacccacaggctcctgcactggccccaccctcccgaggacccccgggacccccaccccacctctcgccctggcctgtggccttgcggtggccctgctcaccctggtgctgctcctggccctgctgcggatgcgcagggtgaaggaagccctgctgcctggtgtccccgacccccgcggctccttccctggcctcttcgagaaacatcatgggaacttccaggcttggatcgcagattctcaggctgctgtccctacggtcccagagcaggacaaagatgatgatgtcatccggcctcagaccaagggggtggaaactcaggaggatgatgatgtcattgccccggggtccccatgccttgggggaggggccctgatgtcggtgggcggggcctcgttcctgatgggagacagcggctacaccaccctgtgacaaccctgtgttgacccctgcctggaccctcgttgctgtcacttatgccccgcttcatttgcataaatatgaatttgttaatctgg

SEQ ID NO: 29 - DNA, 21 bp, Artificial Sequence
  Synthetic Oligonucleotide
tgcctcaccgtgaacttcatg

SEQ ID NO: 30 - DNA, 25 bp, Artificial Sequence
  Synthetic Oligonucleotide
cgtctctctgtgtctagcagaagga

SEQ ID NO: 31 - DNA, 23 bp, Artificial Sequence
  Synthetic Oligonucleotide
tcacctgcacggtttctaaattg

SEQ ID NO: 32 - DNA, 18 bp, Artificial Sequence
  Synthetic Oligonucleotide
cagccgcacgtcatgttg

SEQ ID NO: 33 - DNA, 27 bp, Artificial Sequence
  Synthetic Oligonucleotide
tgacagccgccttttcattttgtttca

SEQ ID NO: 34 - DNA, 19 bp, Artificial Sequence
  Synthetic Oligonucleotide
ggacagctttggtttggga

SEQ ID NO: 35 - DNA, 24 bp, Artificial Sequence
  Synthetic Oligonucleotide
gctagctgctcatttgcatattcg

SEQ ID NO: 36 - DNA, 27 bp, Artificial Sequence
  Synthetic Oligonucleotide
agaagcgctttccatattcatgagccc

SEQ ID NO: 37 - DNA, 20 bp, Artificial Sequence
  Synthetic Oligonucleotide
gggcgacacctcatttgcat

SEQ ID NO: 38 - DNA, 22 bp, Artificial Sequence
  Synthetic Oligonucleotide
gggtctgggtaagatgaactca

SEQ ID NO: 39 - DNA, 21 bp, Artificial Sequence
  Synthetic Oligonucleotide
tcggctcctggatgcttgaca

SEQ ID NO: 40 - DNA, 20 bp, Artificial Sequence
  Synthetic Oligonucleotide
catccgggtcaccaatgatg

SEQ ID NO: 41 - protein, 459 aa, Mus musculus
MMALGRAFAIVFCLIQAVSGESGNAQDGDLEDADADDHSFWCHSQLEVDGSQHLLTCAFNDSDINTANLEFQICGALLRVKCLTLNKLQDIYFIKTSEFLLIGSSNICVKLGQKNLTCKNMAINTIVKAEAPSDLKVVYRKEANDFLVTFNAPHLKKKYLKKVKHDVAYRPARGESNWTHVSLFHTRTTIPQRKLRPKAMYEIKVRSIPHNDYFKGFWSEWSPSSTFETPEPKNQGGWDPVLPSVTILSLFSVFLLVILAHVLWKKRIKPVVWPSLPDHKKTLEQLCKKPKTSLNVSFNPESFLDCQIHEVKGVEARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 42 - DNA, 1380 bp, Mus musculus
atgatggctctgggtagagctttcgctatagttttctgcttaattcaagctgtttctggagaaagtggaaatgcccaggatggagacctagaagatgcagacgcggacgatcactccttctggtgccacagccagttggaagtggatggaagtcaacatttattgacttgtgcttttaatgactcagacatcaacacagctaatctggaatttcaaatatgtggggctcttttacgagtgaaatgcctaactcttaacaagctgcaagatatatattttataaagacatcagaattcttactgattggtagcagcaatatatgtgtgaagcttggacaaaagaatttaacttgcaaaaatatggctataaacacaatagttaaagccgaggctccctctgacctgaaagtcgtttatcgcaaagaagcaaatgattttttggtgacatttaatgcacctcacttgaaaaagaaatatttaaaaaaagtaaagcatgatgtggcctaccgcccagcaaggggtgaaagcaactggacgcatgtatctttattccacacaagaacaacaatcccacagagaaaactacgaccaaaagcaatgtatgaaatcaaagtccgatccattccccataacgattacttcaaaggcttctggagcgagtggagtccaagttctaccttcgaaactccagaacccaagaatcaaggaggatgggatcctgtcttgccaagtgtcaccattctgagtttgttctctgtgtttttgttggtcatcttagcccatgtgctatggaaaaaaaggattaaacctgtcgtatggcctagtctccccgatcataagaaaactctggaacaactatgtaagaagccaaaaacgagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatga

SEQ ID NO: 43 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 44 - DNA, 1380 bp, Homo sapiens
atgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 45 - protein, 459 aa, Artificial Sequence
  mouse/human hybrid Il7ra protein
MMALGRAFAIVFCLIQAVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGGWDPVLPSVTILSLFSVFLLVILAHVLWKKRIKPVVWPSLPDHKKTLEQLCKKPKTSLNVSFNPESFLDCQIHEVKGVEARDEVESFLPNDLPAQPEELETQGHRAAVHSANRSPETSVSPPETVRRESPLRCLARNLSTCNAPPLLSSRSPDYRDGDRNRPPVYQDLLPNSGNTNVPVPVPQPLPFQSGILIPVSQRQPISTSSVLNQEEAYVTMSSFYQNK

SEQ ID NO: 46 - DNA, 1380 bp, Artificial Sequence
  mouse/human hybrid Il7ra mRNA
atgatggctctgggtagagctttcgctatagttttctgcttaattcaagctgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggaggatgggatcctgtcttgccaagtgtcaccattctgagtttgttctctgtgtttttgttggtcatcttagcccatgtgctatggaaaaaaaggattaaacctgtcgtatggcctagtctccccgatcataagaaaactctggaacaactatgtaagaagccaaaaacgagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatga

SEQ ID NO: 47 - protein, 457 aa, Rattus norvegicus
MMALGRAFAIVFCLLQAASGESGNAQDGDLEDAEPDDHSFWCHSQLEVDGNQHSLTCAFNDPDIKTTNLEFQICGALLGIDCLTLNKLREMYFIKTSKFLLIGNSSVCVKLGKMDVICKILDISTIVKPEAPSNLKVVYRKEANDFLVTFNTSHSTKKYVTALKHDVAYRPERGESNWTHVYLFHTRTTILQRKLQPKAVYEIKVRSIPNHEYFKGFWSEWSPSSTFETPDSKYQGGWDPVLPSIILLSLFSMVLLVILAHVLWKKRIKPVVWPSLPDHKKTLEQLCKKPKKNLNVSFNPESFLDCQIHEVNGIQARDEVESFLQNDLPPRPGELEKQGHRATVHGANWPSEISGSTPETFRRESPLRCLARNLSTCNTPAFLSSRSPDYREGDGNRSHVYQDLLPSSRNTNGTVPQPFPLQSGILIPVSQGQPISTSSVLNQEEAYVTMSSFYQNK

SEQ ID NO: 48 - DNA, 3124 bp, Rattus norvegicus
agagctgggtttggtctccccctctctcattcacttgcgcacacaagtgtgcttcttctctcttctctctctcagaatgatggctctgggtagagctttcgctatagttttctgcttacttcaagctgcttctggagaaagtggcaatgcccaggatggagatctagaggatgcggaaccagatgatcactccttctggtgccacagccagctggaagtggatggaaatcagcactcactgacgtgtgcttttaatgacccagacatcaaaaccactaatctggaatttcaaatatgtggggctcttctaggcatagattgcctaactcttaataagctacgagagatgtattttataaagacatcaaaattcttactgattggtaacagcagtgtatgtgtgaagcttggaaaaatggatgtaatttgcaaaattttggacataagcacaatagttaaacctgaggcgccttctaacctgaaagtagtttatcgaaaagaagcaaatgactttttggtaacatttaatacatctcactcaacaaagaaatatgtaacagcattaaagcatgatgtggcctaccgcccagaaaggggtgaaagtaactggacgcatgtatatttattccatacaagaacaacaatcctacagagaaaactacaaccaaaagcagtgtatgaaatcaaagtccgatccattcccaatcatgaatacttcaaaggcttctggagtgagtggagtccaagttctacctttgaaacaccagattccaagtatcaagggggatgggatccggttttgccaagtatcatccttctgagtttgttctctatggttttgttggtcattttagcccatgtgctatggaaaaaaaggattaaacctgttgtatggcctagtcttcctgatcataagaaaactctggaacaactatgtaagaagccaaaaaagaatttgaatgtgagtttcaatcctgaaagtttcctggactgccagattcatgaggtgaatggcattcaagccagggatgaagtggaaagctttctgcaaaatgatcttcctccacggcctggggagctggagaagcagggacatagagcaactgtacacggtgcaaactggccatctgagatttcaggcagcacaccagaaacgttccgaagagaatcacccctaagatgcctggctagaaatctaagcacatgcaatacccctgcattcctttcctctaggtcccctgactacagagaaggtgacggaaataggtctcatgtgtatcaagacttgctgccaagctccagaaacacaaatggcactgtccctcaaccatttcctctccagtcaggaatcctgataccagtttctcaaggacagcccatctctacttcttcagtattgaatcaagaagaagcatatgtcactatgtctagcttttaccaaaacaaatgaattataagaaacctgagaccccttccacagaaaaccaaatgatcactgagatggaaagtctggaatgcttgtctccctcatagctcttagaagagaaagtcaacatggacttgctacacatcttcagcattctaagaaatcattttgatctcctagctcaaaagcatttattcaaagcaggaagaatctgctttccccttgttggattagtcatatgagtacaaatgacccaattaaaattgtaaaactcaattaaatgaagagtaaagggaaagatagaaggaggtgaatacaggaagaagagaaggatgtcagtggtgggtctatcattaggacttactatatatccagcagtacacaacggctctcatttcttcctcacaataatactacaatgtgggttcatccattagaattgttattttctttgtcatagatgctgaagttgaaagtggaaatttttaagtaatgtccaggtttttcttccagcaacagatgaagcatgcattccaacttcaaccctccttggccatgaacctgtcctactactgagtatcaaacatcaccactaagtgggtggttacagtcagaatccaaactgggtcattttggaaagggaaagttagaaaaaattaatagcaagcataaactgtatctttcttagagagatgtggatacatggtcacttcacgtaaagtgtctatgaggatgaacatagaggacaaaatacacttatgggagtgaaataccgtgaccatgtgtcaaaggaagtgggagaaagaaaaaaggcaccaagctcatttgattttgttttctttcatttgaaaacgaacccaaaaagtaataagttataagtcaagaagttccagagtcagttatctagaccatgatcttcctgctgctattacccatcggcttccctgtgagatcgtatggggagctatggccaacctacatcagagcaacatttaacagtgagtagatgtctcctcctgtgacaccattacaccttaccccaagttctacagccttggatattgcctaaactacaggaagaaagggctgtgcacacctcagtgattatcccaactgaaactatgtttgtggaagcataaagaagatgggtaagttactcaaatgcaaatgttgattcatgactgcaagccacaattttgaatccctgctgtgtatggccagtctcctaaagaaaacaacaaataactgaaagacaccgtgattgggtgccttagcattaaaattctttgtttcagtgttgacattggttgtttaaatcggtgtgtctcttcggtcatgtattatatctatgcattatattcagataactacaactgctgctaatgcttgattatatactcaggaaccacatgccatgtaacattactggtttgttctgccatttttcctcttgatatttagaaaggaagaccaaaactcttggccagagacagtatgcaaaacagagatgtcaagaactatgtctaaataatgtgaaatacaatgagaaataggtaacaaatttatcaaccaactatgtctggatccagagaatctctagttattcaatttattttctataagcctttgtctctctcttcatccagacttccatgggaatttttgccttcaaataaaagaatgggcaaatttctggaa

SEQ ID NO: 49 - DNA, 27 bp, Artificial Sequence
  synthetic oligonucleotide
gggatcaatactatgggtggtttataa

SEQ ID NO: 50 - DNA, 20 bp, Artificial Sequence
  synthetic oligonucleotide
acctcagtattctcaagaag

SEQ ID NO: 51 - DNA, 24 bp, Artificial Sequence
  synthetic oligonucleotide
ctacacttgggagtgaaatgcatt

SEQ ID NO: 52 - DNA, 21 bp, Artificial Sequence
  synthetic oligonucleotide
ggagggcactcttacactttc

SEQ ID NO: 53 - DNA, 24 bp, Artificial Sequence
  synthetic oligonucleotide
ttggagaatgacttgcctgctgtc

SEQ ID NO: 54 - DNA, 23 bp, Artificial Sequence
  synthetic oligonucleotide
cctctgcttccttgttcttcaca

SEQ ID NO: 55 - DNA, 22 bp, Artificial Sequence
  synthetic oligonucleotide
cagggcaagcaagaatttagca

SEQ ID NO: 56 - DNA, 28 bp, Artificial Sequence
  synthetic oligonucleotide
tgtgggtattaatcaccaggacagaggg

SEQ ID NO: 57 - DNA, 23 bp, Artificial Sequence
  synthetic oligonucleotide
acaagccatttgcagtattgtca

SEQ ID NO: 58 - DNA, 21 bp, Artificial Sequence
  synthetic oligonucleotide
tgggtcagtttggctatccat

SEQ ID NO: 59 - DNA, 30 bp, Artificial Sequence
  synthetic oligonucleotide
tcttttcccagaacaatgaagatgctatgg

SEQ ID NO: 60 - DNA, 22 bp, Artificial Sequence
  synthetic oligonucleotide
tgctttgggtactgtcctgaag

SEQ ID NO: 61 - DNA, 9475 bp, Artificial Sequence
  Targeted Tslp allele, genomic DAN (total 9475 bp)
  misc_feature (1)..(238): Mouse Sequence
  misc_feature (18)..(20): Start Codon
  misc_feature (18)..(21): Coding Exon 1
  misc_feature (239)..(2166): Human Genomic Fragment 1
  misc_feature (2167)..(6610): Puro Self-Deleting Cassette
  misc_feature (2167)..(2172): Nhel
  misc_feature (2173)..(2198): I_Ceu
  misc_feature (2205)..(2238): LoxP1
  misc_feature (6571)..(6604): LoxP2
  misc_feature (6605)..(6610): Xhol
  misc_feature (6611)..(8782): Human Genomic Fragment 2
  misc_feature (8780)..(8782): Stop Codon
  misc_feature (8783)..(9475): Mouse Sequence
cacgttcaggcgacagcatgggtgactatgggctgtgcagggactgggaaggggtggtgagggctgatacccgagcacagtctttgcaggtctggaggctctccccgcttagaggggcagttccacgggaaacagagttggaactgtgtttcagtgaaaatttttcttactgtgtgtctttccagttcttctcaggagcctcttcatcctgcaagtactagtacggatggggctaacttacgacttcactaactgtgactttgagaagattaaagcagcctatctcagtactatttctaaagacctgattacatatatgagtggggtaagtgaagaagcttttttaaaacaaatgtattttcatcagaggagtcggcatacacacactctacaatttaactttgtaggaaagaaaaataatttagaaaaaatcatggccccacattttgtcaaggattcttacaagtgatattcaaatatctaatctaaaatgattatctagaaattggcacattctaagtgtgcagatgctgatgaggagcaggtattgatagacagcgcgttatgcgtcaaaggatgtctatcctttgctaaagtgttactctgactatgctgtaaaaagcaggaggtaagagcttaagaaagaggagtaaaagagataattctcatgagataaactctaaggattgatgctgtgctccaggtctctccagtgttttagatgtttcaggatgctatttattacagaatatggtgtacttggaattttttttaacatacagtagtaatcattttcctgattaacctaatttctagacagagtttgcattcatgaatggccacagtacagatgcggacatccaaaggatggcattattactcacaagcatagtgctatgtgcagttatggcttgagggaagggaggggggaggtcgccctctgagacctgaaccttttggtgtggtttcaagcactaaccagcactatctaatggctatttcactgccttgtcaatgacataggaaaaaggtacctgagtggaaactgttttcagggcacctttaaagcctgggagcaaagggtggagggatgattttccttgtggacttaaaagtctttaccctctttgtcctatttttctttcttccagaccaaaagtaccgagttcaacaacaccgtctcttgtagcaatcgggtgagtagagagttcagtgctgctggctttctccagggagacgccaggcattttggagagggagtatcctgctacgtgcagaactccgagaggtgcctgggctccgggacgccgccgccgggggaaaggggacatctgggctgtcagagcggggctgcgcctagcttgggacaacacttctgttccaatttagggagaggaagtctctatccggaggaaaggcaaattgggaactgggacgagggaacgttgttaggggcaccacctgctggggtccggcgcctccgcgctcgggctcggaattttggcagcctccgccccctggagacttgggaggagcgagcgtgggtgacagtcttttcgcgacgagtgccctccgccaccctcgccacgcccctgctcccccgcggttggttcttccttgctctactcaaccctgacctcttctctctgactctcgacttgtgttccccgctcctccctgaccttcctcccctcccctttcactcaattctcaccaactctttctctctctggtgttttctccttttctcgtaaactttgccgcctatgagcagccacattgccttactgaaatccagagcctaaccttcaatcccaccgccggctgcgcgtcgctcgccaaagaaatgttcgccatgaaaactaaggctgccttagctatctggtgcccaggctattcggaaactcaggtaagcccgaagcctcagacgtttgctgtaccttggggctaacctcaaattaaactggggctttggtgcagaagtcgttctcttatttttatttaggttttatctttcgaagagcaaacgagccgggtaaaagtggtaggatgtcagttagacccacgttgatacccggaatcaaactcacctatttctacggttctgatactgttttggctgaattatggttctaaaccttagggcaatgtttcaagctatgatgagctagctcgctaccttaggaccgttatagttacctagcataacttcgtatagcatacattatacgaagttatctaggccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagatcatcgatgcatggggtcgtgcgctcctttcggtcgggcgctgcgggtcgtggggcgggcgtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatggtttagttcctcaccttgtcgtattatactatgccgatatactatgccgatgattaattgtcaacacgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccatgatcccgggggatccactagttctagtgtttaaactctagccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattagcggccggccgcctaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctaaaatacacaaacaattagaatcagtagtttaacatcattatacacttaaaaattttatatttacctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggaatttaaatcggtacgcaccttcctcttcttcttgggggttcccatggtgctggcttggccgggagctggctcagagcaggggacaccacctgggtcgagccagccaacctgtgagcaggtggaattttgtgggctgtggcctgggagccagcaccctcttcctcttatagatactagtggcccctaggaattatgaagtcaaagaggaccaggacctcacagaccatggccagtgaggacctgtaccatgtccaaatatgggcatgagaggggtgggcagggctttggcatcaggagttgcttgtgtcacagtcaagaagtgacaaagatggcatccacttgagtgttcagttagtcactcagcttaggtgttaagtgccacacacctgcttctaggctaggtcctgatagataacccaaggccaggcaggtgggtgaaacagccacatggatttgaactgtgaaaagcacacatcttcagactgctcagagaatgctgctgagggaacttgaccttttaagaaattatccaacgccccagtgaggcactgacagacaaatccagagggtctcagagttgcaggggggtgggctctagtaaaacattgaggccccatcaagtgcttcaggtataaatgggagccacatggatgcagagcagtgtttggactgagggaggtgttggacattactagacagaaggtggacgtgggtgctgctactggcatgcatataacttcgtatagcatacattatacgaagttatctcgaggtgagacttctatatcagaatgttttgattgctggagcataagagtatggctgctaaaaatgccaattcccaggtactcaacccagaccttcaacattaaaatctcagattatggggctccttaagagattcttgtccagtccaaagtttgagcaacacctcttgttcttatcacttaattattgtgtgcttatttgctaaatgtataattacattatacataaaatctctatcctatgtttgcttaattgcttgtgtgggcgctattgctgtctctttacacatttttgcacatgtagttatctgcatttgaatgctcgtgtagcattaaatatggagatagtgtagtggaaagttaggcacaggaactctggagacaacctgcctgactttgaatcctggccctataacttctgtgaagacttagttaaattacttagcctccgtgtactgtagcttcatgggtaaagtaagtatcatatcagttagtcttatacaggttgtttctgaggattaaattagtcaacacatgtaaatgcagttggaacagtgcctggtacacaacaggcactcaatatttatttcagtcagcaagtagaggatttatcttcatggtgacaagtttaaggaacagagagagacaagtgcagatatgtttgattgctccttattagcctagtggactttatatgtctacagtctaggtagatggacacgactgtcactttttttttttttttttttttgagacagaatctcgctctgttacccaggctggaatgcagtggcacgatctcagctcactgcaacctccatctcctgggttcaagcctcagcctcccgagtagctggaactacaggtgcccgccaccacgcctggctaacttttgtatttttagtagagacggggtttcaccatattggccaggctggtctcgaattcctgaccttgtgatctgcctgcctcggcctcccaaagtgctggaattacaagcatgagccaccatgcccagccaaaactgtcactttctagaggttgaggattgaagccatagcgctgatctgggttgagcttgaattagaaactcaataccagacagccatatgggaaacctatttggcttcatgccttcttatgaaggagaccctggcaaatctgcagatggctacaataaaattcatttaaataagagcacaaacaaaaagctagatcaagttcttggacagcatgtgagaaagggagagtttggagaaatttatttcagtccctcccaagcccaaatggagagtctaagactaataataatgattttgcaggtttttttaagatttgtgcttaataaccctgtgactttattaatttgcataccatgtgtctaggaggcccagtgtactactcaaaggtaattcagataaaggtatatactgcaatcctctttaaaataagccctcagatgtctgtgacacatctagacaatggggcaggggagggggaaggatggggagcaggagcatgcattttgggtccaaaaaatagactaggtttattgaatgatgtctataaacaggtataagatagctcttgcccatgaggaacttgtgatcttgtcagggaggtcttgaaatcagcaatttattcatttacttaatcactcaacaaatattcagtgtttcctatgattaagacactgtattcagtgctatggggaatacctatgatgcaatataaagaaaagcatgttaagtgagagccaagttaaatgacacacactcttaagtactggaagagtttccaaaagcaaggtctgagcaattagtggaggctttttgaaggaggtggtgcttggccttgaagcaaaagtaggtgggtacagaaacaggaaggcattcccctggaaaaggcacatgctagcacatagtaagcaggtgctttggagacacactgaaagatggatttgcatagagaaggcaattaaacctgctctcaacagttactaaagatagtgaaaagtaattttgactattgattcttatattctgcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaattagctttcagcttctgctatgaaaatctctatcttggttttagtggacagaatactaagggtgtgacacttagaggaccactggtgtttattctttaattacagaagggattcttaacttattttttggcatatcgcttttttcagtataggtgctttaaatgggaaatgagcaatagaccgttaatggaaatatctgtactgttaatgaccagcttctgagaagtctttctcacctcccctgcacacaccttactctagggcaaacctaactgtagtaggaagagaattgaaagtagaaaaaaaaaattaaaaccaatgacagcatctaaaccctgtttaaaaggcaaggatttttctacctgtaatgattcttctaacattcctatgctaagattttaccaaagaagaaaatgacagttcgggcagtcactgccatgatgaggtggtctgaaagaagattgtggaatctgggagaaactgctgagatcatattgcaaatccagctgtcaaagggttcagacccaggacagtacaattcgtgagcagatctcaagagccttgcacatctacgagatatatatttaaagttgtagataatgaatttctaatttattttgtgagcacttttggaaatatacatgctactttgtaatgaatacatttctgaataaagtaattctcaagtttg

SEQ ID NO: 62 - DNA, 5109 bp, Artificial Sequence
  Targeted Tslp allele (without cassette), genomic DNA (total 5109 bp)
  misc_feature (1)..(238): Mouse Sequence
  misc_feature (18)..(20): Start Codon
  misc_feature (18)..(21): Coding Exon 1
  misc_feature (2167)..(6610): Deleted Puro Self-Deleting Cassette
  misc_feature (2167)..(2172): Nhel
  misc_feature (2173)..(2198): I_Ceu
  misc_feature (2205)..(2238): LoxP
  misc_feature (2239)..(2244): Xhol
  misc_feature (2245)..(4416): Human Genomic Fragment 2
  misc_feature (4414)..(4416): Stop Codon
  misc_feature (4417)..(5109): Mouse Sequence
cacgttcaggcgacagcatgggtgactatgggctgtgcagggactgggaaggggtggtgagggctgatacccgagcacagtctttgcaggtctggaggctctccccgcttagaggggcagttccacgggaaacagagttggaactgtgtttcagtgaaaatttttcttactgtgtgtctttccagttcttctcaggagcctcttcatcctgcaagtactagtacggatggggctaacttacgacttcactaactgtgactttgagaagattaaagcagcctatctcagtactatttctaaagacctgattacatatatgagtggggtaagtgaagaagcttttttaaaacaaatgtattttcatcagaggagtcggcatacacacactctacaatttaactttgtaggaaagaaaaataatttagaaaaaatcatggccccacattttgtcaaggattcttacaagtgatattcaaatatctaatctaaaatgattatctagaaattggcacattctaagtgtgcagatgctgatgaggagcaggtattgatagacagcgcgttatgcgtcaaaggatgtctatcctttgctaaagtgttactctgactatgctgtaaaaagcaggaggtaagagcttaagaaagaggagtaaaagagataattctcatgagataaactctaaggattgatgctgtgctccaggtctctccagtgttttagatgtttcaggatgctatttattacagaatatggtgtacttggaattttttttaacatacagtagtaatcattttcctgattaacctaatttctagacagagtttgcattcatgaatggccacagtacagatgcggacatccaaaggatggcattattactcacaagcatagtgctatgtgcagttatggcttgagggaagggaggggggaggtcgccctctgagacctgaaccttttggtgtggtttcaagcactaaccagcactatctaatggctatttcactgccttgtcaatgacataggaaaaaggtacctgagtggaaactgttttcagggcacctttaaagcctgggagcaaagggtggagggatgattttccttgtggacttaaaagtctttaccctctttgtcctatttttctttcttccagaccaaaagtaccgagttcaacaacaccgtctcttgtagcaatcgggtgagtagagagttcagtgctgctggctttctccagggagacgccaggcattttggagagggagtatcctgctacgtgcagaactccgagaggtgcctgggctccgggacgccgccgccgggggaaaggggacatctgggctgtcagagcggggctgcgcctagcttgggacaacacttctgttccaatttagggagaggaagtctctatccggaggaaaggcaaattgggaactgggacgagggaacgttgttaggggcaccacctgctggggtccggcgcctccgcgctcgggctcggaattttggcagcctccgccccctggagacttgggaggagcgagcgtgggtgacagtcttttcgcgacgagtgccctccgccaccctcgccacgcccctgctcccccgcggttggttcttccttgctctactcaaccctgacctcttctctctgactctcgacttgtgttccccgctcctccctgaccttcctcccctcccctttcactcaattctcaccaactctttctctctctggtgttttctccttttctcgtaaactttgccgcctatgagcagccacattgccttactgaaatccagagcctaaccttcaatcccaccgccggctgcgcgtcgctcgccaaagaaatgttcgccatgaaaactaaggctgccttagctatctggtgcccaggctattcggaaactcaggtaagcccgaagcctcagacgtttgctgtaccttggggctaacctcaaattaaactggggctttggtgcagaagtcgttctcttatttttatttaggttttatctttcgaagagcaaacgagccgggtaaaagtggtaggatgtcagttagacccacgttgatacccggaatcaaactcacctatttctacggttctgatactgttttggctgaattatggttctaaaccttagggcaatgtttcaagctatgatgagctagctcgctaccttaggaccgttatagttacctagcataacttcgtatagcatacattatacgaagttatctcgaggtgagacttctatatcagaatgttttgattgctggagcataagagtatggctgctaaaaatgccaattcccaggtactcaacccagaccttcaacattaaaatctcagattatggggctccttaagagattcttgtccagtccaaagtttgagcaacacctcttgttcttatcacttaattattgtgtgcttatttgctaaatgtataattacattatacataaaatctctatcctatgtttgcttaattgcttgtgtgggcgctattgctgtctctttacacatttttgcacatgtagttatctgcatttgaatgctcgtgtagcattaaatatggagatagtgtagtggaaagttaggcacaggaactctggagacaacctgcctgactttgaatcctggccctataacttctgtgaagacttagttaaattacttagcctccgtgtactgtagcttcatgggtaaagtaagtatcatatcagttagtcttatacaggttgtttctgaggattaaattagtcaacacatgtaaatgcagttggaacagtgcctggtacacaacaggcactcaatatttatttcagtcagcaagtagaggatttatcttcatggtgacaagtttaaggaacagagagagacaagtgcagatatgtttgattgctccttattagcctagtggactttatatgtctacagtctaggtagatggacacgactgtcactttttttttttttttttttttgagacagaatctcgctctgttacccaggctggaatgcagtggcacgatctcagctcactgcaacctccatctcctgggttcaagcctcagcctcccgagtagctggaactacaggtgcccgccaccacgcctggctaacttttgtatttttagtagagacggggtttcaccatattggccaggctggtctcgaattcctgaccttgtgatctgcctgcctcggcctcccaaagtgctggaattacaagcatgagccaccatgcccagccaaaactgtcactttctagaggttgaggattgaagccatagcgctgatctgggttgagcttgaattagaaactcaataccagacagccatatgggaaacctatttggcttcatgccttcttatgaaggagaccctggcaaatctgcagatggctacaataaaattcatttaaataagagcacaaacaaaaagctagatcaagttcttggacagcatgtgagaaagggagagtttggagaaatttatttcagtccctcccaagcccaaatggagagtctaagactaataataatgattttgcaggtttttttaagatttgtgcttaataaccctgtgactttattaatttgcataccatgtgtctaggaggcccagtgtactactcaaaggtaattcagataaaggtatatactgcaatcctctttaaaataagccctcagatgtctgtgacacatctagacaatggggcaggggagggggaaggatggggagcaggagcatgcattttgggtccaaaaaatagactaggtttattgaatgatgtctataaacaggtataagatagctcttgcccatgaggaacttgtgatcttgtcagggaggtcttgaaatcagcaatttattcatttacttaatcactcaacaaatattcagtgtttcctatgattaagacactgtattcagtgctatggggaatacctatgatgcaatataaagaaaagcatgttaagtgagagccaagttaaatgacacacactcttaagtactggaagagtttccaaaagcaaggtctgagcaattagtggaggctttttgaaggaggtggtgcttggccttgaagcaaaagtaggtgggtacagaaacaggaaggcattcccctggaaaaggcacatgctagcacatagtaagcaggtgctttggagacacactgaaagatggatttgcatagagaaggcaattaaacctgctctcaacagttactaaagatagtgaaaagtaattttgactattgattcttatattctgcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaattagctttcagcttctgctatgaaaatctctatcttggttttagtggacagaatactaagggtgtgacacttagaggaccactggtgtttattctttaattacagaagggattcttaacttattttttggcatatcgcttttttcagtataggtgctttaaatgggaaatgagcaatagaccgttaatggaaatatctgtactgttaatgaccagcttctgagaagtctttctcacctcccctgcacacaccttactctagggcaaacctaactgtagtaggaagagaattgaaagtagaaaaaaaaaattaaaaccaatgacagcatctaaaccctgtttaaaaggcaaggatttttctacctgtaatgattcttctaacattcctatgctaagattttaccaaagaagaaaatgacagttcgggcagtcactgccatgatgaggtggtctgaaagaagattgtggaatctgggagaaactgctgagatcatattgcaaatccagctgtcaaagggttcagacccaggacagtacaattcgtgagcagatctcaagagccttgcacatctacgagatatatatttaaagttgtagataatgaatttctaatttattttgtgagcacttttggaaatatacatgctactttgtaatgaatacatttctgaataaagtaattctcaagtttg

SEQ ID NO: 63 - DNA, 20473 bp, Artificial Sequence
  Targeted Tslpr allele 7558, genomic DNA, total 20473 bp
  misc_feature (1)..(1051): Mouse Sequence
  misc_feature (111)..(113): Start Codon
  misc_feature (111)..(189): Coding Exon 1
  misc_feature (1052)..(5860): Neo Self-Deleting Cassette
  misc_feature (1052)..(1057): Xhol
  misc_feature (1058)..(1091): LoxP1
  misc_feature (5790)..(5823): LoxP2
  misc_feature (5829)..(5824): I_Ceu
  misc_feature (5855)..(5860): Nhel
  misc_feature (5861)..(19603): Human Sequence
  misc_feature (19604)..(20473): Mouse Sequence
  misc_feature (20336)..(20338): Stop Codon
gcccccggcttcccgttttcggctctaagcggcctgggcgccctcgactcggaccggctcggaccgaaccagctgtcaatcactgcagcgtccgcggccccgccggcgacatggcatgggcactcgcggtcatcctcctgcctcggctccttgcggcggcagcggcggcggcggcggtgacgtcacggggtgaggagtgagcgggggcggggctgcctgtcaatcgccgcggtgggcggggcccgagcaagagctaccaagttgcttttcgtcccatcattgcttttcgtcccatcatgaatatgcaaataaggcctctggccctcctaagggcgatcggatagcgcttcgtttgcatattcattttgatctttgcgtatgcatgagccccgccctccccccctacgctcggcgctttctcctcagtaatatgcaaatgagacctaaaccccgccttgacctcattagcatagtgctgccgccacaatctcgctcctcctcctgaatatgcaaataaggcctctgggccgctccttctttgcatattcatatacagctttcccgcttatatgcaaataacgcttcgcccctaccgagttctcactcatcgcttctcatttgcatatccatcgggagatacacatattcatgagcgatgtatttctgtcttccatcccctcatgaatatagaattgatgccctgtccatatggatcactatgcatttgcatattttccccacgatttacatatgcacaagcctcaacgtcttgctccaacgtccctgatcatgaatatgcagatgagacctcagatctccgaaattgaatctgcccgctcctcatcatatactgctattctcatatgcaccagccacaaagtcttccatccttttcccctcatgaatatgcaaagaatgcttccccagtccatctccactctggttcactgcctgctcatttgtgtatccattggtcgctttgcatagtggtgagccccgcttccctaccgctttctttgcgatcatgtatattcaaatgaggctccgacttgtcgagataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggtaaatataaaatttttaagtgtataatgatgttaaactactgattctaattgtttgtgtattttaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggcggccggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctagtaactataacggtcctaaggtagcgagctagccatttctgaagttggtgtgtgtggtgttttgagaaagacagtaaggtggatcgatggggacagtttccgatgccaccttaggtatccacaggggatgtgttgttggatacatgattgatctatacagatacatctgtgcatggcactagtgggtgaacttaccttttgcaaaaacaaaaacaaaagcagccgggcgcggtggctcacgcctgcaatctcagcactttaggtggatgaggcgggcggatcacgaggtcaggagatcgagaccatcctggctaacacagtgaaaccccatctctactaaaagtacaaaaaattatccaggcatggtggcgggcacctgtagccccagctactcgggaggctgaggcaggagaatggcgtgaacccgggaggcggagcttgcagtgagccgagattgcgccactgcagtccagcctgggtgacagagtgagagtctgtctcacacaatacactacactacactacactacactacactacactacactacactacaatacaatgcaatacaatgcaatacaatacgtcaggcgcggtggctcacgcctgtaatcccagtactttgggaggccgaggcgggtggatcatgaggtcaggagatcgagaccatcctggctaacacggtgaaaccccgtctctactaaaaatacaaaaaattagccaggtgtggtgacaggtgcctgtaatcccggctactcgggaggctgaggcaggggaatcgcttgaacccgggaggcggaggttgcagtgagccgagatcgtgccattgtactccagcctgggcaacagagtgagactctgtctcaaaaagaaaaataaatttaaaaaaataaaaaaatagagttgcctcaccgtgaacttcatgcgtctctctgtgtctagcagaaggagtacagattcagatcatctacttcaatttagaaaccgtgcaggtgacatggaatgccagcaaatactccaggaccaacctgactttccactacaggtaagtggcccccagaaagcgaaccccacgcccagggggctcaaaatcgctcttctgagccgtcagcgattgggaatgcttgagaacaaaagtgtaaagcatcagactgtgcaagccggaatgtttgttctgtgaaaccctcagccctcaccacaaccacaatctcattgccccaaaccttcttgccaagggatggctgactcactgtcaaaatcaactacattcttttttttgtttgtttttattttgagtcggagtctcactccgttgccacgctggagtgcagtggcgcaatctcggctcactgcaacctctgcctcccgggttcaagccattcccctgcctcagcctcccgagtagctgggattacaggcacctgccaccatgcctagctaatttttgtatttttagtagagatggtgattcaccatattggtcaggccggtttcaaactcctgacctcatgtgatccacctgcctcagcctcccaaagtgctgggatgacaggcgtgagccacggcatccggactgaaaatcacctagattcttcacgggagattaacagagagttttgttcacccagagctctgggtgaactttaagtcttatctgctaggcagccggggagggaaagactcagtctctgaaacatttcacagccttacccggggctacccccaaccctgtgtctccctgatgtgactttatttttatttttactttggcttagttagacacccagaagtatgcgtacgaacattgcaggaagggtttttccaataaatttaatcatgggtggatgattcttcagtttgataacaaaagagataaacggcaaagcgtacagaacagggaaattttaaaacgaagttatttggaaaataagactcaccgttgggagaccgaggtgggtggatcatctgagatcaggagttccagaccatcctggccaacatggtgaaaccccaactctactaaaaatacaaaaactagccgggcgtggcggcaggtgcctgtaatcccagctactcaggaggctgaggcaagagaatcgcttcaacctgggagacggagttcacggtgagccgagattgcactccagcctggccgacagagcgagactgtctcaaaaacaaaacaaaacaaaacaaaaacctcacaaaaagaaaaaaaaaagtgctggattttgcacgtagttgcagaattcagctcagtttcttcctctgtaaaaggggcagttcttggggggggtgttggctcatgcctgttatcccagcactttgggaggctgaggggggggggggtggatcacctgaggtcaggagttcaagaccagcctggccaacatggtgaaacctcgtctctactaaaaatacaaaaaaattagccgggcgcgggggcgtgcgcctgtaatcccagcaacttgggaggctgaggcaggagaatcgcttgaatccgggaggcagaggttgcagtgggccgagatcacgccattgcactccagcctgggcaacaagaacaaaactctgtttcaaaaaacaaacgaacaaataaaaaaacggccgggcgcggtggctcacgcctgtaatcccagcactctgggaggccgaggcgggtggatcacctgaggtcaggagatcgagaccagcctggtcaacatggcgaaatcccatctctattaaaaacacaaaaattagctgggggtggtggtgcgtgcccgtaatcccagctactcaggaggcagaggcaggagaatcgcttgaacccgggaggcagagattgcaatgagccgagatcgtgccactgcactccagcctcagggacagagcgagacaccatcttaaaaaaaaaaaaaaaaaaaaagccggtgtggtagctcacacctctcattccagcagtttgggaggccaaggtgtatggatcacctgaggtcaggagttccagaccagcctggccccaacatggtgaaaccctgtctccagtaaaactacaaaaattagctgtatatggtggcaggtgcctgtaatcccagctactcaggaggctgagacaggagaattgcttgaacccgggaggcagaggttgcagtgagctgagatcgcgccattgcactccaacctgggcgacaagagcaagaccccatctctaaataaataagacatgcttttttgttttgttgctgaatggtcatcgttttaaaccacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattaccgtaagtattgtaaagccagctcaccatgcttttcagtacttccttcagcttattaccacaaggactgaaaaccaagctcatgcaaatcgccggatccatgattaccgttaactattgagaagcaagctcaccatgcctttcagtacttcctgcagcttattactgtaagtaacgaaaaaccaagctccagccagtcgcgggatcaatgattcccgtaactattgaaaaggaagctcagcatagttttcagtacttccttcagcttattaccgtaagtactgaaaagctcacgtaaatcgtcggatccatgattaccgtaataattgaaaagcaagctcagcacgcttttcactacttccttcagctgattaccgtaagtaccgaaaagcaatctcatgatagtcgctggatcaatgattaccgtaaccactgaaaagcaacccagcctacttttcgctacttccttcaggttattaccataagtactgaaaagctcatgcaaattaccggatccatgattaccataactattgaaaagcaagctcaccatgcttttcgtacttccttcagcttattaccataagtactgataaagcaagctcatgcaagtcactggatcagtgattaccgtagctattgaaaagcaagctcaccatgcttttcagtacttccttcagcttattaccataagtactgaaaagcaagctctagcaagtcgcaggatcagattcccgtaactattgaaaagcagcccagcatccttttcactacttccttcagcttattgccataaataaaaaagctcatgcaaataaccggatccatgattaccgtaactattgaaaagcaagctcaacatgcttttcagtatttccttcagcttattaccataagtactgaaaagcaagctctcgcaagtcgcgggatcaatgattaccataacaattgaaaagcaagctcagcatgcttttcagtacttccttcagctgattatcataagtaccgaaaagcaagctctcgcaagtcgcgggatcaatgattaccgtaacgattgaaaagcaatcccagcacacttttcagtaccccttcatctccttaccttcagttactttcagttaagttctgaaaatcaagctcatggaccattggccactagagcccggtgcccagctcctcactccaagtgggaagaaaccggccttccaggaagttccctcttacacgcacactggttggggattgaatctgcccccagtggggagaccagatgaccccgggagactgtgatttaagggaatgaattaaaggcggggcgcggtggctcacgcctgtaatcccagcactatgggaggccaaggcgggcggatcacttgaggtctggagttcgagaccagcctcaccaacatggtgaaaccccatctctacaaacaaacaaacaaacaaaaaattagccgggctggtggcgcatgcctgtaatcctagctgttcgggaggctgaggcagggaaattgcttgaacctgggaggcagaggctgcagtgagccacgttggtgccactgcactccagcctgggcgatttataaattcattatttaaaaataaataaggcccagggtggtggcttacccctgtaatcccagtactttgggagaccaaggcgggcggatcacttgaggtctggagttcgagaccagccttgccaacgtggtgaaaccccgtctctattaaaaataatttaaaaaaaattagccaggcgtggtggcacacgcttgtaatcccagctactcgggaggctgaggcagggaaattgcttgaacccgggaggcggaggctgcagtgagccaagacggtgccactgcactgcagcctgggcgatttataaatttattatttaaaaataaataaggcccagggcggtggctctcccctgtaatcccagtactttgggaggccaaggcagggggatcacgtgacgtggggagttcgagaccagcctgaccaacatggagaaactccatctctattaaaaatacataattagccgggcttggtggtgcatgcctgtaatcccagctactcgggacgctgaggcaggagaatctcttgaatctgggaggcggagtttgtggtgagccgagatcgcgccattgcactccagcctggacaacaagagtgaaactccatctcaaataaataaattcattaaattaaataagggaattaattagagatgctctctggtgccctgcctacacacacacacacacacacacacacacacacacacacacacacacagagtgagctggaaatactctctcatcctcatcccactcactggatgttctctctctttttttttttttttttgagagagagggtcttcctctgttgcccaggctggttttcttatgtgcgttgagaactggctgttaagtctcgggcgaggaaatgagggacaaatgtagggaaaccctgtttccaaaatgtttattctttatcctagaattctgtaaggctgtgtttcttttttacttttttattttttagaggtaggggaaacggatctgtttgagaatccggtgagaactatgaactctttattcgcgaaattacttacagatactgggtgcgtttcctgggctataataggtcaccacaaactggaggcttaaaacagcagaaatttattctctcccagttttgaagcccatgagtctgagatggagatgtctgagagccgcattccctctggaggttctaagggaggatccttcctgcctctcccagctcctgggggctccaggcatccctgggcttgtggccgcatcactccagtctctgcctccgtctccatgtggccttctcctctgtgtctcctcttctgtctcttacaagggcacctgtcattagatttaggggacaccctactccaggatgatctcacctcaaggtccttcacctaattacatctgcagagagcctatttccaaatccggtctcattccaggtcctgggctttaggatgtggacagatgtttctgggggccactgttccattcagtataattatattcagttccttccagggttctaggggaggctccttcctacctctcccagctcctgggggctccaggcatccctgggcttgtggccccatcactccagtctctgcctccgtctccacgtggcctcctcctctgtgtctgcgtctcctcctctgtctccgagaaggacacctgtcattggatttagaacccttccttctccagtatgacctcatcctaactaactgaatctataatgatcctaactaactgaatctataatgatcctttttttttttttttttttttttgagatagtctcgctctgtcacccaggctggagggcagtggttcaatctcggctcaccgcaacctccgcctccccggttcaagcgattctcctgcctcagcctcactaatagctgggattacaagcgctggccaccatgcctgtatttttagtagagacggggttttagcatattggccaggctggtctcgaactcctgacttcaggtgatccacccgcctcggccttctaaaggatcctatttttaaatacagttccattctgagtttctggggagttgggattttaacatatatttttgtgggggacttaatttagcccgtaacagacacacaggacattttccgcagaattttagagagttccttccttcacaaacctacaggttctcggagctctggtggaagcttctcctataaaagtaatgaggacgggtgaaccccgagacccataaggtattaaggggaggaggggtacaggctagagaaaggggatgaggtcagcctgtcacaatcagctcagagaggagggacgtcgcttccgttatttcttcttctcagtgaaacccagttccccgaagcacgtgagattttcgtggcatcaggatgcagtgacggtgacgtgttctgacctgtcctacggggatctcctctatgaggttcagtaccggagccccttcgacaccgagtggcaggtgagccgggcggccgcgactcagggcgatggtggctgagcgtcccccaggtgcgggctgtgggattcgctgtttcatcagacctcgctcccctctgtctacaccttcctgaattccactctgctgtatcttcctgagagagctctagtccagcttggctttttcatgtttctctctgtgtctctgaggggtctccagagaaagagaaccaataatatgtctgtctgtctgtctatcaatctatttatctatctatctatcaatctatctatatcattctattttatctctacctctatctatgtatctatatcattctatttccctctctttcaatctatcatctatctatctcattctattttatctctatctctgtctttcaatctatctcattctattttatctctatctctgtctttcaatctatcatctattttatctattaactctctttgttctatctatctctctatggctctatccatatctattttatctaccaactctcttttatctatctatgtatggtcaatcttctatatatatcatccctatctctatgtagttcaactatctatcatctctatctaactacccattatctctattctaccatctactatcttatctatgtatttatctatctatgtatgtatctatacatctatcatctatccgtctctatctatctaagtatcatctatctatctactctgtatcatctgtctgtctgtctctctatctatctaatgtatcatctatttatctctctatcatctgtctatgtatcatctatctctctatcattgctctatcatctatgtatcatctatctctctctcatcactctatcatctatgtatcatctatctatctctctatcatctatctatgtatcatctgtctctctctcatcgctctatcatctatgtatcatctatctatctatcatctgtctatgtatcatctatcaactctgtcatctctctgttttatctatgtatctatcatttctttatctgtctacctctagtcctatctctatctgtatctctacacacctgtctctctacacacacacacacacacacagacacacaaacacaggcacacagacacacacagacatgcacagacacacacagacacacacagacacacacaggcacacacacacagtcatgtgctgcctaacgaccttttggtcatcagcagactgcatgtatcacggtagtctcctatgattatcacacagctgtcctatgcagctgtcccaattattttcttctatatcatatttttcctgtaccttctctatgtttagatacacaaatacttacccttgcgttaggtttgtctgcagtattcagtacagtaacgtgctgtatggctgtgtagctgaagagcaatttactatacagcctaggtgtgcagcgggctagaccagctaggtgtgtgtaagtaaactctagaatattctcataatgaagaaatcacctcacaatgaatttcccagaacatgtccccatcgttaagcaatgcatgactgtatatctacctatatgtgtgtgtacacacacacatatacacacacatatgtacgtaccttgtgtacacagcttatatatacatatatacacacgcatatacacacatatagatacacagcttatatacacatatatatacacatgcatatacacacatatagatacacagcttatatacacacatatatacataccttatatatgcatatttatacacatgtatatacacatcttatatacgcacacatatacacacacacacagcttatatacacatatatatacccacacatatatacacagcttatatacacgtatatatatgtatatatgcaccttatatacacagcttatatacacatatttatacacacgtatacacacaccttatataaacacatatatacacacatatacacagcttatataacatatatacacacacatatatacataccttatatatgcatatttatacacatgtacatacacatcttatatacacatatatatacatacatatgtgcacagcttatatacacgtatatatacacatatatatgcaccttatatacacagcttatatacacatatttatacacacgtatacacacaccgtatatacacacacatacacacatatacacagcttatatacacagcttatatacaggtatatatacacacatgtatatacacacatgtatatatacacaaactttatatatatatacacacacagcttatatacaatacatatacacacatatatacacaccttatatacacacatatatacacacacatatatacagagcttatatacacagcttatatacacatatatatacacacgtatatacacacctaatatacacagcttatatatacacatatatacacacatacatatatacacaacacacgtacacagcttatatatacacatatatacacacacatatagatacatagcttatatatacacatatatacacatcttatatacatagcttctatacacacagatatacacacaccttatacacacaacttatatatacacaattatatatacacaccttatatatacagcttatatatacacacatatataaatatacatatagagatatcttattggtacatatatctacaaacatatataaactatatatgtataaactgatacatagaatagaacattcatctgtatctttatatccatttatatctgtaaagatatgtagatacaggctggatgcagtggctcacacctttaatcccagcactttgggaggccgaggagggtggatcacctgaggtcaggagttcaagaccagcctggccaacgtggtgaaacctcatctctactagaaatacaaaaattagccaagcatggtggtgcctgtaatccgagctactcgggaggctgaggcacaagaattgcttgaacccgacaggcagaggttgcagtgagccgagaccgcaccactgcactccagcctgggcaacagagcaagactctgtctcagtaaataaataaataatatattaaaataataataaagtaaatacaggccaggcacagtggctcatgcctgtaatcccagtactgttggaggccaaggcaggaggatcgcttgagcccaggagttgttgaccagcttgggcaacagagtgagaccccatctctttttctttttttagacacagtcccgctctgtcacccaggctggagtgcagtggtgcgatctctgcttgctacaaccttcgcctcccaggttcaagcgattctcctgcctcagcctcccaagtagctgggattacaggcacccgccaccacgcctggctaatttttgtattatcagtagagacggggtttctccatgttggccaggctggtctcgaacttgcgacctcaggtgatccacccgcctcagcctcccaaagcgttgggtttacaggagtgagccactgtgtccggcctcgcggctccattcttgaagtcagcgagactgtgaaccctccggaaggaaaactctggacagacaggttacatcactaatcattgtgtgtgtgtgtgtgtgtgtgtgtgtgtgtgtgtgtgtgtgtgtgtatgtgtatgtttctccagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcggggtaatgcttgttacacggcagtgtcccatagccttgtcaccaggctggagtaggcatagccactgccttcccgggaggtgggagggagggtgtcctgccttcgaggtgggagggagggtgtcctgccttcctgggaggtgggagggagggtgtcctgccttcgaggtgggagggagggtgtcctatcttcccgggaggtgggagggagggtgtcctatcttcccgggaggtgggagggaggctgtcctgccttcgaggtgggagggagggtgtcctgccttcctgggaggtgggagggagggtgtcctgccttcctaggaggtgggagggagggtgtcctgccttcctgggaggtgggagggagggtgtcctgccttcgaggtgggagggagggtgtcctgtcttcccgggaggtgggagggagggtgtcctatcttcccgggaggtgggagggaggctgtcctgccttcgaggtgggagggagggtgtcctgccttcctgggaggtgggagggagggtgtcctgccttcctaggaggtgggagggagggtgtcctgccttcctgggaggtgggagggagggtgtcctgccttcgaggtgggagggagggtgtcctgccttcctgggaggtgggagggagggtgtcctgccttctaggtgggagggagggtgtcctgccttcctgggaggtgggagggagggtgtcctgccttcgaggtgggagggagggtgtcctgccttcctgggaggtgggagggagggtgtcctgccttctaggtgggagggagggtgtcctgccttcctgggaggtgggagggagggtgtcctgccttcgaggtgggagggagggtgtcctgccttcctgggaggtgggagggagggtgtcctgccttcgaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcgaggtgggagggagggtgtcctgccttcccaggagggtgtgtctgagcaagctcagccttctgtcatcctccagggccccatctgaaagtaattcctctccttgtttcattctcgggctgctttcacttgggagagttttctttcttttgtttttttgagaaacattctcatggtgcccaggctggagtgcagtggcgcgatctcggctccctgcaacatctgtctcccacgttcaatcgattctcccgcctcagcctcccgagtgcctgggattacagtgtacacgccaccacacctgattaaatctttttattttttatttttttatttttatttttttttagagggagtctcagtctgtggtccaggctggagttcagtggcacggtcttggctcactgcaacctccgcctcccgagttcaagtgattctcctgcctcagcctcccaagtagctgggactacaggcacgcaccaccaagcccgcctaatgtttgtatttttagtagggatggcgtttccccatattggccaggctggtctcgaactcctgaccttgtgatccgcccgcctcggcctcccaaagtgctgggattaaaggcgtgagccaccgcgcccggcctttgacgtatgttttcagggggcatgatttaaggagcacagccaccaacacacgaaagcaagctctcatatgctgtaaacacctccagcaaccagaagcctccaaaggctacatgttggacctgcccttacttcctcggggtggctgtgggatgtgttgttgtccagagacacggtgtctctttccaggatattttccataggaatgaaaatttgctgacactaacattcattaaaaaaaaaaaataggtccatctctactaaaaatacaaaaattagcccggtgtggtggcggatgcctgtcatcccagctgctcaggaggctgagacaggagaatcgcttgagtccgggagacggaggttgcagtgagctgagatggagccactgcactccagcctgggcaacagagtgaggttctgcctcaaaaaaaaaaaaaaaaaaaagtaaaagatttgctgacacgaatatttacaaaaaacaaaaacaaaacaaaaaacaaacaacgagaaccaagaaaaaaaaacgaacaccaaaaaatgagggagactggttagggatgagatgtaacatcacgttgaaaacgtacagccgcacgtcatgttgaaaactgacagccgccttttcattttgtttcagatgcctgtgcagagacaccaacgcctcccaaaccaaagctgtccaaactcctgcccctgggctgcggcctagcagcgctgctgacactgtccctgctcctggccgccctgaggcttcgcaggtgaggggtctccgaggagtcatggggtcatggtggggtaattgcgatgtctacggcgatgacgtcaccactgtgatgacgtcatgttcgggtcgggggcatggcatgggggtgtcaggtgaaccctgacccctgaccctgaaccccagggtgaaagatgcgctgctgccctgcgtccctgaccccagcggctccttccctggactctttgagaagcatcacgggaacttccaggtgcgcggggggggggggggggtcaggatcgctgtggggggtcacttcctgtccccggagagtgagggcatgaggcagggggatgatgggagtgacaggcgtcacgggccactgcctgactccaggcaccgctcccctccccctccccccacccccttccctccaccccccccctccacacacacaacccctttcgctgcaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgct

SEQ ID NO: 64 - DNA, 15741 bp, Artificial Sequence
  Targeted Tslpr allele 7559 (without cassette), genomic DNA, total 15741 bp
  misc_feature (1)..(1051): Mouse Sequence
  misc_feature (111)..(113): Start Codon
  misc_feature (111)..(189): Coding Exon 1
  misc_feature (1052)..(1128): Deleted Neo Self-Deleting Cassette
  misc_feature (1052)..(1057): 5Xhol
  misc_feature (1058)..(1091): LoxP
  misc_feature (1097)..(1122): I_Ceu
  misc_feature (1123)..(1128): Nhel
  misc_feature (1129)..(14871): Human Sequence
  misc_feature (14872)..(15741): Mouse Sequence
  misc_feature (15604)..(15606): Stop Codon
gcccccggcttcccgttttcggctctaagcggcctgggcgccctcgactcggaccggctcggaccgaaccagctgtcaatcactgcagcgtccgcggccccgccggcgacatggcatgggcactcgcggtcatcctcctgcctcggctccttgcggcggcagcggcggcggcggcggtgacgtcacggggtgaggagtgagcgggggcggggctgcctgtcaatcgccgcggtgggcggggcccgagcaagagctaccaagttgcttttcgtcccatcattgcttttcgtcccatcatgaatatgcaaataaggcctctggccctcctaagggcgatcggatagcgcttcgtttgcatattcattttgatctttgcgtatgcatgagccccgccctccccccctacgctcggcgctttctcctcagtaatatgcaaatgagacctaaaccccgccttgacctcattagcatagtgctgccgccacaatctcgctcctcctcctgaatatgcaaataaggcctctgggccgctccttctttgcatattcatatacagctttcccgcttatatgcaaataacgcttcgcccctaccgagttctcactcatcgcttctcatttgcatatccatcgggagatacacatattcatgagcgatgtatttctgtcttccatcccctcatgaatatagaattgatgccctgtccatatggatcactatgcatttgcatattttccccacgatttacatatgcacaagcctcaacgtcttgctccaacgtccctgatcatgaatatgcagatgagacctcagatctccgaaattgaatctgcccgctcctcatcatatactgctattctcatatgcaccagccacaaagtcttccatccttttcccctcatgaatatgcaaagaatgcttccccagtccatctccactctggttcactgcctgctcatttgtgtatccattggtcgctttgcatagtggtgagccccgcttccctaccgctttctttgcgatcatgtatattcaaatgaggctccgacttgtcgagataacttcgtataatgtatgctatacgaagttatgctagtaactataacggtcctaaggtagcgagctagccatttctgaagttggtgtgtgtggtgttttgagaaagacagtaaggtggatcgatggggacagtttccgatgccaccttaggtatccacaggggatgtgttgttggatacatgattgatctatacagatacatctgtgcatggcactagtgggtgaacttaccttttgcaaaaacaaaaacaaaagcagccgggcgcggtggctcacgcctgcaatctcagcactttaggtggatgaggcgggcggatcacgaggtcaggagatcgagaccatcctggctaacacagtgaaaccccatctctactaaaagtacaaaaaattatccaggcatggtggcgggcacctgtagccccagctactcgggaggctgaggcaggagaatggcgtgaacccgggaggcggagcttgcagtgagccgagattgcgccactgcagtccagcctgggtgacagagtgagagtctgtctcacacaatacactacactacactacactacactacactacactacactacactacaatacaatgcaatacaatgcaatacaatacgtcaggcgcggtggctcacgcctgtaatcccagtactttgggaggccgaggcgggtggatcatgaggtcaggagatcgagaccatcctggctaacacggtgaaaccccgtctctactaaaaatacaaaaaattagccaggtgtggtgacaggtgcctgtaatcccggctactcgggaggctgaggcaggggaatcgcttgaacccgggaggcggaggttgcagtgagccgagatcgtgccattgtactccagcctgggcaacagagtgagactctgtctcaaaaagaaaaataaatttaaaaaaataaaaaaatagagttgcctcaccgtgaacttcatgcgtctctctgtgtctagcagaaggagtacagattcagatcatctacttcaatttagaaaccgtgcaggtgacatggaatgccagcaaatactccaggaccaacctgactttccactacaggtaagtggcccccagaaagcgaaccccacgcccagggggctcaaaatcgctcttctgagccgtcagcgattgggaatgcttgagaacaaaagtgtaaagcatcagactgtgcaagccggaatgtttgttctgtgaaaccctcagccctcaccacaaccacaatctcattgccccaaaccttcttgccaagggatggctgactcactgtcaaaatcaactacattcttttttttgtttgtttttattttgagtcggagtctcactccgttgccacgctggagtgcagtggcgcaatctcggctcactgcaacctctgcctcccgggttcaagccattcccctgcctcagcctcccgagtagctgggattacaggcacctgccaccatgcctagctaatttttgtatttttagtagagatggtgattcaccatattggtcaggccggtttcaaactcctgacctcatgtgatccacctgcctcagcctcccaaagtgctgggatgacaggcgtgagccacggcatccggactgaaaatcacctagattcttcacgggagattaacagagagttttgttcacccagagctctgggtgaactttaagtcttatctgctaggcagccggggagggaaagactcagtctctgaaacatttcacagccttacccggggctacccccaaccctgtgtctccctgatgtgactttatttttatttttactttggcttagttagacacccagaagtatgcgtacgaacattgcaggaagggtttttccaataaatttaatcatgggtggatgattcttcagtttgataacaaaagagataaacggcaaagcgtacagaacagggaaattttaaaacgaagttatttggaaaataagactcaccgttgggagaccgaggtgggtggatcatctgagatcaggagttccagaccatcctggccaacatggtgaaaccccaactctactaaaaatacaaaaactagccgggcgtggcggcaggtgcctgtaatcccagctactcaggaggctgaggcaagagaatcgcttcaacctgggagacggagttcacggtgagccgagattgcactccagcctggccgacagagcgagactgtctcaaaaacaaaacaaaacaaaacaaaaacctcacaaaaagaaaaaaaaaagtgctggattttgcacgtagttgcagaattcagctcagtttcttcctctgtaaaaggggcagttcttggggggggtgttggctcatgcctgttatcccagcactttgggaggctgaggggggggggggtggatcacctgaggtcaggagttcaagaccagcctggccaacatggtgaaacctcgtctctactaaaaatacaaaaaaattagccgggcgcgggggcgtgcgcctgtaatcccagcaacttgggaggctgaggcaggagaatcgcttgaatccgggaggcagaggttgcagtgggccgagatcacgccattgcactccagcctgggcaacaagaacaaaactctgtttcaaaaaacaaacgaacaaataaaaaaacggccgggcgcggtggctcacgcctgtaatcccagcactctgggaggccgaggcgggtggatcacctgaggtcaggagatcgagaccagcctggtcaacatggcgaaatcccatctctattaaaaacacaaaaattagctgggggtggtggtgcgtgcccgtaatcccagctactcaggaggcagaggcaggagaatcgcttgaacccgggaggcagagattgcaatgagccgagatcgtgccactgcactccagcctcagggacagagcgagacaccatcttaaaaaaaaaaaaaaaaaaaaagccggtgtggtagctcacacctctcattccagcagtttgggaggccaaggtgtatggatcacctgaggtcaggagttccagaccagcctggccccaacatggtgaaaccctgtctccagtaaaactacaaaaattagctgtatatggtggcaggtgcctgtaatcccagctactcaggaggctgagacaggagaattgcttgaacccgggaggcagaggttgcagtgagctgagatcgcgccattgcactccaacctgggcgacaagagcaagaccccatctctaaataaataagacatgcttttttgttttgttgctgaatggtcatcgttttaaaccacagattcaacggtgatgaggcctatgaccagtgcaccaactaccttctccaggaaggtcacacttcggggtgcctcctagacgcagagcagcgagacgacattctctatttctccatcaggaatgggacgcaccccgttttcaccgcaagtcgctggatggtttattaccgtaagtattgtaaagccagctcaccatgcttttcagtacttccttcagcttattaccacaaggactgaaaaccaagctcatgcaaatcgccggatccatgattaccgttaactattgagaagcaagctcaccatgcctttcagtacttcctgcagcttattactgtaagtaacgaaaaaccaagctccagccagtcgcgggatcaatgattcccgtaactattgaaaaggaagctcagcatagttttcagtacttccttcagcttattaccgtaagtactgaaaagctcacgtaaatcgtcggatccatgattaccgtaataattgaaaagcaagctcagcacgcttttcactacttccttcagctgattaccgtaagtaccgaaaagcaatctcatgatagtcgctggatcaatgattaccgtaaccactgaaaagcaacccagcctacttttcgctacttccttcaggttattaccataagtactgaaaagctcatgcaaattaccggatccatgattaccataactattgaaaagcaagctcaccatgcttttcgtacttccttcagcttattaccataagtactgataaagcaagctcatgcaagtcactggatcagtgattaccgtagctattgaaaagcaagctcaccatgcttttcagtacttccttcagcttattaccataagtactgaaaagcaagctctagcaagtcgcaggatcagattcccgtaactattgaaaagcagcccagcatccttttcactacttccttcagcttattgccataaataaaaaagctcatgcaaataaccggatccatgattaccgtaactattgaaaagcaagctcaacatgcttttcagtatttccttcagcttattaccataagtactgaaaagcaagctctcgcaagtcgcgggatcaatgattaccataacaattgaaaagcaagctcagcatgcttttcagtacttccttcagctgattatcataagtaccgaaaagcaagctctcgcaagtcgcgggatcaatgattaccgtaacgattgaaaagcaatcccagcacacttttcagtaccccttcatctccttaccttcagttactttcagttaagttctgaaaatcaagctcatggaccattggccactagagcccggtgcccagctcctcactccaagtgggaagaaaccggccttccaggaagttccctcttacacgcacactggttggggattgaatctgcccccagtggggagaccagatgaccccgggagactgtgatttaagggaatgaattaaaggcggggcgcggtggctcacgcctgtaatcccagcactatgggaggccaaggcgggcggatcacttgaggtctggagttcgagaccagcctcaccaacatggtgaaaccccatctctacaaacaaacaaacaaacaaaaaattagccgggctggtggcgcatgcctgtaatcctagctgttcgggaggctgaggcagggaaattgcttgaacctgggaggcagaggctgcagtgagccacgttggtgccactgcactccagcctgggcgatttataaattcattatttaaaaataaataaggcccagggtggtggcttacccctgtaatcccagtactttgggagaccaaggcgggcggatcacttgaggtctggagttcgagaccagccttgccaacgtggtgaaaccccgtctctattaaaaataatttaaaaaaaattagccaggcgtggtggcacacgcttgtaatcccagctactcgggaggctgaggcagggaaattgcttgaacccgggaggcggaggctgcagtgagccaagacggtgccactgcactgcagcctgggcgatttataaatttattatttaaaaataaataaggcccagggcggtggctctcccctgtaatcccagtactttgggaggccaaggcagggggatcacgtgacgtggggagttcgagaccagcctgaccaacatggagaaactccatctctattaaaaatacataattagccgggcttggtggtgcatgcctgtaatcccagctactcgggacgctgaggcaggagaatctcttgaatctgggaggcggagtttgtggtgagccgagatcgcgccattgcactccagcctggacaacaagagtgaaactccatctcaaataaataaattcattaaattaaataagggaattaattagagatgctctctggtgccctgcctacacacacacacacacacacacacacacacacacacacacacacacagagtgagctggaaatactctctcatcctcatcccactcactggatgttctctctctttttttttttttttttgagagagagggtcttcctctgttgcccaggctggttttcttatgtgcgttgagaactggctgttaagtctcgggcgaggaaatgagggacaaatgtagggaaaccctgtttccaaaatgtttattctttatcctagaattctgtaaggctgtgtttcttttttacttttttattttttagaggtaggggaaacggatctgtttgagaatccggtgagaactatgaactctttattcgcgaaattacttacagatactgggtgcgtttcctgggctataataggtcaccacaaactggaggcttaaaacagcagaaatttattctctcccagttttgaagcccatgagtctgagatggagatgtctgagagccgcattccctctggaggttctaagggaggatccttcctgcctctcccagctcctgggggctccaggcatccctgggcttgtggccgcatcactccagtctctgcctccgtctccatgtggccttctcctctgtgtctcctcttctgtctcttacaagggcacctgtcattagatttaggggacaccctactccaggatgatctcacctcaaggtccttcacctaattacatctgcagagagcctatttccaaatccggtctcattccaggtcctgggctttaggatgtggacagatgtttctgggggccactgttccattcagtataattatattcagttccttccagggttctaggggaggctccttcctacctctcccagctcctgggggctccaggcatccctgggcttgtggccccatcactccagtctctgcctccgtctccacgtggcctcctcctctgtgtctgcgtctcctcctctgtctccgagaaggacacctgtcattggatttagaacccttccttctccagtatgacctcatcctaactaactgaatctataatgatcctaactaactgaatctataatgatcctttttttttttttttttttttttgagatagtctcgctctgtcacccaggctggagggcagtggttcaatctcggctcaccgcaacctccgcctccccggttcaagcgattctcctgcctcagcctcactaatagctgggattacaagcgctggccaccatgcctgtatttttagtagagacggggttttagcatattggccaggctggtctcgaactcctgacttcaggtgatccacccgcctcggccttctaaaggatcctatttttaaatacagttccattctgagtttctggggagttgggattttaacatatatttttgtgggggacttaatttagcccgtaacagacacacaggacattttccgcagaattttagagagttccttccttcacaaacctacaggttctcggagctctggtggaagcttctcctataaaagtaatgaggacgggtgaaccccgagacccataaggtattaaggggaggaggggtacaggctagagaaaggggatgaggtcagcctgtcacaatcagctcagagaggagggacgtcgcttccgttatttcttcttctcagtgaaacccagttccccgaagcacgtgagattttcgtggcatcaggatgcagtgacggtgacgtgttctgacctgtcctacggggatctcctctatgaggttcagtaccggagccccttcgacaccgagtggcaggtgagccgggcggccgcgactcagggcgatggtggctgagcgtcccccaggtgcgggctgtgggattcgctgtttcatcagacctcgctcccctctgtctacaccttcctgaattccactctgctgtatcttcctgagagagctctagtccagcttggctttttcatgtttctctctgtgtctctgaggggtctccagagaaagagaaccaataatatgtctgtctgtctgtctatcaatctatttatctatctatctatcaatctatctatatcattctattttatctctacctctatctatgtatctatatcattctatttccctctctttcaatctatcatctatctatctcattctattttatctctatctctgtctttcaatctatctcattctattttatctctatctctgtctttcaatctatcatctattttatctattaactctctttgttctatctatctctctatggctctatccatatctattttatctaccaactctcttttatctatctatgtatggtcaatcttctatatatatcatccctatctctatgtagttcaactatctatcatctctatctaactacccattatctctattctaccatctactatcttatctatgtatttatctatctatgtatgtatctatacatctatcatctatccgtctctatctatctaagtatcatctatctatctactctgtatcatctgtctgtctgtctctctatctatctaatgtatcatctatttatctctctatcatctgtctatgtatcatctatctctctatcattgctctatcatctatgtatcatctatctctctctcatcactctatcatctatgtatcatctatctatctctctatcatctatctatgtatcatctgtctctctctcatcgctctatcatctatgtatcatctatctatctatcatctgtctatgtatcatctatcaactctgtcatctctctgttttatctatgtatctatcatttctttatctgtctacctctagtcctatctctatctgtatctctacacacctgtctctctacacacacacacacacacacagacacacaaacacaggcacacagacacacacagacatgcacagacacacacagacacacacagacacacacaggcacacacacacagtcatgtgctgcctaacgaccttttggtcatcagcagactgcatgtatcacggtagtctcctatgattatcacacagctgtcctatgcagctgtcccaattattttcttctatatcatatttttcctgtaccttctctatgtttagatacacaaatacttacccttgcgttaggtttgtctgcagtattcagtacagtaacgtgctgtatggctgtgtagctgaagagcaatttactatacagcctaggtgtgcagcgggctagaccagctaggtgtgtgtaagtaaactctagaatattctcataatgaagaaatcacctcacaatgaatttcccagaacatgtccccatcgttaagcaatgcatgactgtatatctacctatatgtgtgtgtacacacacacatatacacacacatatgtacgtaccttgtgtacacagcttatatatacatatatacacacgcatatacacacatatagatacacagcttatatacacatatatatacacatgcatatacacacatatagatacacagcttatatacacacatatatacataccttatatatgcatatttatacacatgtatatacacatcttatatacgcacacatatacacacacacacagcttatatacacatatatatacccacacatatatacacagcttatatacacgtatatatatgtatatatgcaccttatatacacagcttatatacacatatttatacacacgtatacacacaccttatataaacacatatatacacacatatacacagcttatataacatatatacacacacatatatacataccttatatatgcatatttatacacatgtacatacacatcttatatacacatatatatacatacatatgtgcacagcttatatacacgtatatatacacatatatatgcaccttatatacacagcttatatacacatatttatacacacgtatacacacaccgtatatacacacacatacacacatatacacagcttatatacacagcttatatacaggtatatatacacacatgtatatacacacatgtatatatacacaaactttatatatatatacacacacagcttatatacaatacatatacacacatatatacacaccttatatacacacatatatacacacacatatatacagagcttatatacacagcttatatacacatatatatacacacgtatatacacacctaatatacacagcttatatatacacatatatacacacatacatatatacacaacacacgtacacagcttatatatacacatatatacacacacatatagatacatagcttatatatacacatatatacacatcttatatacatagcttctatacacacagatatacacacaccttatacacacaacttatatatacacaattatatatacacaccttatatatacagcttatatatacacacatatataaatatacatatagagatatcttattggtacatatatctacaaacatatataaactatatatgtataaactgatacatagaatagaacattcatctgtatctttatatccatttatatctgtaaagatatgtagatacaggctggatgcagtggctcacacctttaatcccagcactttgggaggccgaggagggtggatcacctgaggtcaggagttcaagaccagcctggccaacgtggtgaaacctcatctctactagaaatacaaaaattagccaagcatggtggtgcctgtaatccgagctactcgggaggctgaggcacaagaattgcttgaacccgacaggcagaggttgcagtgagccgagaccgcaccactgcactccagcctgggcaacagagcaagactctgtctcagtaaataaataaataatatattaaaataataataaagtaaatacaggccaggcacagtggctcatgcctgtaatcccagtactgttggaggccaaggcaggaggatcgcttgagcccaggagttgttgaccagcttgggcaacagagtgagaccccatctctttttctttttttagacacagtcccgctctgtcacccaggctggagtgcagtggtgcgatctctgcttgctacaaccttcgcctcccaggttcaagcgattctcctgcctcagcctcccaagtagctgggattacaggcacccgccaccacgcctggctaatttttgtattatcagtagagacggggtttctccatgttggccaggctggtctcgaacttgcgacctcaggtgatccacccgcctcagcctcccaaagcgttgggtttacaggagtgagccactgtgtccggcctcgcggctccattcttgaagtcagcgagactgtgaaccctccggaaggaaaactctggacagacaggttacatcactaatcattgtgtgtgtgtgtgtgtgtgtgtgtgtgtgtgtgtgtgtgtgtgtgtatgtgtatgtttctccagtccaaacaggaaaatacctgcaacgtcaccatagaaggcttggatgccgagaagtgttactctttctgggtcagggtgaaggctatggaggatgtatatgggccagacacatacccaagcgactggtcagaggtgacatgctggcagagaggcgagattcggggtaatgcttgttacacggcagtgtcccatagccttgtcaccaggctggagtaggcatagccactgccttcccgggaggtgggagggagggtgtcctgccttcgaggtgggagggagggtgtcctgccttcctgggaggtgggagggagggtgtcctgccttcgaggtgggagggagggtgtcctatcttcccgggaggtgggagggagggtgtcctatcttcccgggaggtgggagggaggctgtcctgccttcgaggtgggagggagggtgtcctgccttcctgggaggtgggagggagggtgtcctgccttcctaggaggtgggagggagggtgtcctgccttcctgggaggtgggagggagggtgtcctgccttcgaggtgggagggagggtgtcctgtcttcccgggaggtgggagggagggtgtcctatcttcccgggaggtgggagggaggctgtcctgccttcgaggtgggagggagggtgtcctgccttcctgggaggtgggagggagggtgtcctgccttcctaggaggtgggagggagggtgtcctgccttcctgggaggtgggagggagggtgtcctgccttcgaggtgggagggagggtgtcctgccttcctgggaggtgggagggagggtgtcctgccttctaggtgggagggagggtgtcctgccttcctgggaggtgggagggagggtgtcctgccttcgaggtgggagggagggtgtcctgccttcctgggaggtgggagggagggtgtcctgccttctaggtgggagggagggtgtcctgccttcctgggaggtgggagggagggtgtcctgccttcgaggtgggagggagggtgtcctgccttcctgggaggtgggagggagggtgtcctgccttcgaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcctgggaggtgggagggagggtgtcctgccttcgaggtgggagggagggtgtcctgccttcccaggagggtgtgtctgagcaagctcagccttctgtcatcctccagggccccatctgaaagtaattcctctccttgtttcattctcgggctgctttcacttgggagagttttctttcttttgtttttttgagaaacattctcatggtgcccaggctggagtgcagtggcgcgatctcggctccctgcaacatctgtctcccacgttcaatcgattctcccgcctcagcctcccgagtgcctgggattacagtgtacacgccaccacacctgattaaatctttttattttttatttttttatttttatttttttttagagggagtctcagtctgtggtccaggctggagttcagtggcacggtcttggctcactgcaacctccgcctcccgagttcaagtgattctcctgcctcagcctcccaagtagctgggactacaggcacgcaccaccaagcccgcctaatgtttgtatttttagtagggatggcgtttccccatattggccaggctggtctcgaactcctgaccttgtgatccgcccgcctcggcctcccaaagtgctgggattaaaggcgtgagccaccgcgcccggcctttgacgtatgttttcagggggcatgatttaaggagcacagccaccaacacacgaaagcaagctctcatatgctgtaaacacctccagcaaccagaagcctccaaaggctacatgttggacctgcccttacttcctcggggtggctgtgggatgtgttgttgtccagagacacggtgtctctttccaggatattttccataggaatgaaaatttgctgacactaacattcattaaaaaaaaaaaataggtccatctctactaaaaatacaaaaattagcccggtgtggtggcggatgcctgtcatcccagctgctcaggaggctgagacaggagaatcgcttgagtccgggagacggaggttgcagtgagctgagatggagccactgcactccagcctgggcaacagagtgaggttctgcctcaaaaaaaaaaaaaaaaaaaagtaaaagatttgctgacacgaatatttacaaaaaacaaaaacaaaacaaaaaacaaacaacgagaaccaagaaaaaaaaacgaacaccaaaaaatgagggagactggttagggatgagatgtaacatcacgttgaaaacgtacagccgcacgtcatgttgaaaactgacagccgccttttcattttgtttcagatgcctgtgcagagacaccaacgcctcccaaaccaaagctgtccaaactcctgcccctgggctgcggcctagcagcgctgctgacactgtccctgctcctggccgccctgaggcttcgcaggtgaggggtctccgaggagtcatggggtcatggtggggtaattgcgatgtctacggcgatgacgtcaccactgtgatgacgtcatgttcgggtcgggggcatggcatgggggtgtcaggtgaaccctgacccctgaccctgaaccccagggtgaaagatgcgctgctgccctgcgtccctgaccccagcggctccttccctggactctttgagaagcatcacgggaacttccaggtgcgcggggggggggggggggtcaggatcgctgtggggggtcacttcctgtccccggagagtgagggcatgaggcagggggatgatgggagtgacaggcgtcacgggccactgcctgactccaggcaccgctcccctccccctccccccacccccttccctccaccccccccctccacacacacaacccctttcgctgcaggcctggattgcggacgcccaggccacagccccgccagccaggaccgaggaggaagatgacctcatccacaccaaggctaagagggtggagcccgaggacggcacctccctctgcaccgtgccaaggccacccagcttcgagccaagggggccgggaggcggggccatggtgtcagtgggcggggccacgttcatggtgggcgacagcggctacatgaccctgtgaccttgaagtcactgccagtctatacttcaggctgaggtcacttcctgtctttaaataattcaaactcacaaatcctgtgcctgtctgtatgcaaatgtggtcacaaatattcaaataaaatgcaaatgctatgct

SEQ ID NO: 65 - DNA, 26915 bp, Artificial Sequence
  Targeted Il7ra allele, genomic DNA (total 26915 bp)
  misc_feature (1)..(184): Mouse Sequence
  misc_feature (117)..(119): Start Codon
  misc_feature (185)..(310): Human Genomic Fragment 1
  misc_feature (311)..(5528): Hyg Self-Deleting Cassette
  misc_feature (317)..(350): LoxP1
  misc_feature (5458)..(5491): LoxP2
  misc_feature (5529)..(22634): Human Genomic Fragment 2
  misc_feature (22635)..(26915): Mouse Sequence
  misc_feature (25217)..(25219): Stop Codon
acagagctggtttgggtctccctctctctcattcacttgcacatacaagcgtgcttcttctctattctttctctctctctctctctctctctctctctctctctctctctctcagaatgatggctctgggtagagctttcgctatagttttctgcttaattcaagctgtttctggagaaagtggctatgctcaaaatggtgagtcatttctaagttttcttatggattttggattatctgtagcatggtttcaggttattcagttccctaacagacctgagtcaggcactgggtttgaatgcagtttgaggtcgagataacttcgtataatgtatgctatacgaagttatatgcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgaaaaagcctgaactcaccgcgacgtctgtcgagaagtttctgatcgaaaagttcgacagcgtgtccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgattgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagcccgggcgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggtacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagtgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggtaaatataaaatttttaagtgtataatgtgttaaactactgattctaattgtttgtgtattttaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgatctagataagtaatgatcataatcagccatatcacatctgtagaggttttacttgctttaaaaaacctcccacacctccccctgaacctgaaacataaaatgaatgcaattgttgttgttaaacctgccctagttgcggccaattccagctgagcgtgcctccgcaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggaataacttcgtataatgtatgctatacgaagttatgctagtaactataacggtcctaaggtagcgagctagcaatttcccacatattcagtcattttttttaatgtttaaccaccatgacagggggcaggggatcaatactatgggtggtttataagacctcagtattctcaagaaggaatgcatttcactcccaagtgtagatcttaaatgttgaatgattactctgctcttacaaaaagaatgctcatgtagatgctatgactgtacttgtaggaaaatgtccaaagtaattttaccttgtcaggagatcaaactggattcattttgtttgactttttaagaaatcctgaaagcataactttcaggataaggtaatgtacagaagcaatagctttgtcttcagtgaccagtgctatatcctcagcacctaaatcagtggctagaatatagtagacatccaataacttttgaaagtgttttcaaaatactttagttttgagagatttatgtgagattttaagtaaataactgactagagaaagatctaaatgagtttactcattgaaatacactgaattgcctccacaccaacaaattggccatatgtaataattctttttgggatctaaaaaacttagtaccgagaagccaaccctgcccatacataaacacattgtaattataacaaaactaggcagaagcttctaacagcagcaggaggcatgtgggaatttagaccatcaacttgctcctgcaaattaagccctttctctttaagagttaaaaactatttggctatagacaatatcaaacacatcagcctaatgactcagcttatgcattttgagtcatgtaattacgaaggatggaaatccctagaattttctcattaagggaattgtcagagagtttgacattttttacagtatatgactcactttatgggggatgattattattctatgctaaactttgccttggatttccacaaagactgatgggaggcaggaaacataaatcttactctctttcatgtcatctatactcactagttcaccctggtgatcatactatttttaaaatatataagaatgctagttgaaagctgggttttcactccaactttttaagtttcagattttttagaagatgtataattaccctattcacatgattacgtcaaaatacttcccagtttggggtataggaattcacattcagttgctgcttgttgaaagttgtcaattttctgatcatcacaaggatgatcaagagaagaaagggatactttttaaaaatccaaatcatttacactattaatcaactaactccattcagtaggaagaagacttctagatgacactggcttgcctatgatacatattccacacaatttaaatttttatggataaatatgtctagatacctatttaaatatgaataatattaattattgagcatttaaagaataatagattaactcattattcaaaagctctatgtaatttcaaaaccatagtaattataacaccgtcaattgacataaactttttaaagagaagctcaaatgtttcatgtatattttcagaattagaattcttattttaccttttcattacttatttctcagaaaatattatactcatagctaatccctattaaatccttactgtgttctaagctacctctttgtaaatatccattcagtgattgctcatagcacgagtttacatattagaacacatgtcttagagaagttgcctacctgacagaggaccacaggtagagtatccagaatttaaacgcacatctgtccagctctaacaccacaggtcttaaccactgtgtacattaactactcttagccaagaatttttcagctcacgtcatgtagaatattctttttgtaaaatgccatcacattttataagtcattgaagggaatttttcttggttacaaagcaactctgccccataatatctactgaaaagccagtgagctgcttcctaaaacacagccattttaggtgcaggaaacagtgtataaatggctcattgtatattgtatgctttgccagactgagtggcagtgggagtcctttgttatgtgggtgctgacatctgctagagtgtgctgtctctattgaagaatcgtgaagacaaagccgacccacaggatgtctgaatccaaataataatacatgttctgtgtatagaattggtggaagagaaaatgtcaggacagtgtgaggactgccatgtaaggtcagaaccactgcatttagaaagctaccactgcacagggaagaaatctaagtctacaaaattagtgggctgtctctcattatttcgtgctgtcatcagaaggagggccataccctgctgaaactacataaagagcttttgctggtggcagaactgtgaactggatggattctgggaatggccagaaaaacaaatgcctgtggttgtgagcagtgcccacacccatggtctagctagggctgtttgagatttgttgctttgactgaaccaacctgtcattcaactggttggtccattcacagtcagctttattaactttcccattttccctactgagttatttaagtaaagaaagtgctattcggacagcccttggtctctgggacaatcaactgggatttgattttagtatattctgtctccagtgtaaagccttggaagcatctaatttctagtactgatgaaccaaaaatacatggaagcagtcctaggctcacacttgagcactctgagaatggctttgcttactccagattttctcaggtcccagtgggtgtatattttctgacatatttattccagcctcactttctatcatgtaaaacatacatacaaaatgtagatttcattatagggtctacaaaacagcttaagaaaccaaatactatgtgtgacagatcacactttccaaaagtaatagcaaaaaaaaaaaaaatctggttccccactttcttccagcatcctgctagaatctatcagatactgcgtctatagaagaatctataagaacagaagcagtatgtacaacattcacaggaagtttcaccaaatcggagtcctgccagatctaattttttttccctaatcacgtttgtctcagtcagtagcttaagacaatggaaataatcagtgccacttttaattgggatgcctttttaggcaagggaaagtgacctcttaaaaaagcaaaattctgactgcaagatagctatcattgtccttcatttaagacaaaaaaaatactagggagggaataaattatgatttgtaataaagtgaaaagtgagattaggtagcatggggataatggaaataaagtgtctcttctttgaaataatatgaacaatcaatgtaacaaatgtagcagaaaaaactccagtttaaatacagaaaagaatgtgttcaatgcctctggttctttaactcagaaatatttggaggttacttactcattatgatggattttttttttctattggaaaactctgttagcattgagcgtttttgttttttgttttttgttggttggttggttttgaagcatttttcttgtctttgcccttgggcttttcttccttgaatactacataatccattactatttcatgtctgccacagagtctgctattttattaaggtcatgccatatttcaaaaggatgcatttatttgtttcattaacagctgcatgtttgttcctccccaggagacttggaagatgcagaactggatgactactcattctcatgctatagccagttggaagtgaatggatcgcagcactcactgacctgtgcttttgaggacccagatgtcaacatcaccaatctggaatttgaaatatggtgagggatggtggttttaatggttgcttagacatcctctgtctctcttttcatatgctctttttaatagccacaaaagaaagaatatgtggcctaattaacaaatgttaacatctaaggaattcccaaaggcctcctgaaactccttgtccttcaccaaaaacactcatacaaatctcctctcacggttcagctttcagaccctgagactcagtcaaatgatgctctggatcttggggatcccacatccctcccaacttcatatcagaatttaaatcctgcgtctcctacaacacttctcaccaaaaatctgtttgcccaacacgagacaatccagtgtcttcaagttgcatctgagagttaaactgccttgtttccaatcccaataccagtgcttactagttttttgacctagagaaagttatgtaatgtatctatgcctcagtttcctcacctgtaaaatgagataacctgcctcacaggaaggctgtgatggttaaataatttcatcatataaatcattccaaatagtcggccagtgaataacgagtaatggggaagcaacattaaattataattctgtgaatattgacctaacttctaccatcttgacacaatttgacttcagatgatcctctcaatgtaaattttccaaaaatccacaggaataagttggcattttgtttcacaaggtctcacagaaaagacaaaggaaaagagtctggtttgaaagtttactaaaggtctcagggaactttatcttctccttctccttcatccataagtcatctcttgttgccaagggttactatctctggtgatttgagaaactactctagcttgaaattctgacctgaggctatctccaaattcatatccgaatgacctactttttagttagtgtcctagtgagcaaagtaaatcaagatccaccagtagtaatagaaggcttcctacattccatagacactgagacaattctccacagtctatagtccaaacaagccctgaattccagtttttgtcaatttatgggagcttcctgcatctatttatggagtgctttctgctgcagtccttagataaacatgctgttggacttgagtagtgtactgtgttctctgtctgcctctgttcacttccctaacacattttccaggaataaaatatgtcaaaagaacctgaaccagttcgatgtccacaatctaggctggaaatggattgcactaaaacagccataacaactcattcaaacaaggcactcattttcatgggcaaatcactctcccacacggaggtttgactttggcttctttaaccagctggctggtgggctgagtgttcatcctggtttctcttggccaagctgaggttgacctttctgttcactttcattcacaccatatttgaccacttccttgcccactcaaacatacttaccctttaacatatctcttgacttttcctgtcatattgtaatctgtccagagcctcctctatttgggttttccaattggattcagatatttcagttggaaagggactgccttaagaaagaaacgttttcagtggaaaatatatgtatgagctctttaatagatgaactcctggagttcagagcccttaaaaggatgcccagtttcacaagacagccatacggtcatccttgattgtccattgctcattaatttcattctcaaaatcatgggaatgagctgagaataccattttagatcctccttaaattcccaacagtaccagaaacttgctacaggttggggcctgtaattggatatttcacacatactttccttacaaatatattctatactcaagaattgaactaaaagttattgtcctagtttctccacatcccatgtttacctaaaattcagaaatgggaccccgctcccagtctccccttctatatttatttatcaaatcgtgacaacattaccatcttcagatctttccacctgatgtttgtcctaagcttattccctggtatctgtctagcttacccaaaaattcggtttttatttttatcctgttccaagttgggaaagcctatctaccccaacaaggaacacaactccctagtaactttgagacacacacacacatacacacctactctttaaagcctaaacaatcgcacactctaaaagatagcagttaacaaaagtaacgatttgggagaacagttttaaggaatgtccccaaaataatcaatacatttagccagttaattaacttaacatttcttcaccaatctctagttttcatgactgtaggagcttaaccagtcactctcagaccacaataaaccaaaggtgaaagattctgtaacaaaagctagggcactctcccctgcatttaacctcctggccagctcactcgaagccagacaaacaggttcctctttttgtgcagagtccaggaaccattctcgaaaggactcatttgagcacatgcagagaagagtgtacacacatccagttcaccaagggaagccaacacacattgtgggttgtaggtagtaaaaggccttcctagaacacactccttaggatttaaacaaaattacatcggttaatggaaagaattctttcatatacgcaaacttacccagaggaacttttcttctgcccagatcttcacttccaatttgacccagttatacctctttagagctatttggctgagcttaaacagcacataggaaaaacaaattggtaactgtgtttatcacagaagaggaaaattaaatttagggttgggaaaggaaaataaccctatgatattacttttattctacctttacaatgagaatatatacctttgttacttctttaatttttacattatttacttatttttctttgctttcttgtttgattacaatgcattttaggggtaaaatttatgtgtggtaaaatgcacaaaaattaagtgaatttggagaaatgtctatgacctgtagccattccaatggtaaagatatagaacttatttttcccctagaaggatgcttcatgttcctttccagtcaatcttcataccccaggagcaatcataattctcaattctattaccctttggtttttgccagtttctgatagttcttattaatagaatactctttattcttttctgtcttctttcatttaaccagtgtttgtgagagttagccatgttgatgtccatctcatagctcatcttttcaattgctaagtagtaattccactgtatgaatataccacaaatttttaattctttctcttcttgatgaacatttgtgttttttcaagtttgagactattattttttaggttgctgttcacattcttggacaaatcagtttgtgtatatatattttcatttttctggggtataaaacctcagaatggaattgctgtgtcataaggtaagcatgtatctaagtttataagaaaccgcccaacagtttttcaaagtggttataccattctactctccttccagcgatgcatgagagatatacatcatttgcaacgtttgactttgggatagtatctcgttaggtttttaattcgcatttgtcaaataacaaatgttgagcagcttttcatatacttggtcttttgcctgtcttctttgggctagtatctgttaaaagcactgagttatttgtccttttgttattgctggatatgagttctttatacattctgtatacatttcctttgtcagatagatgtattgcatctattttctattctgaagtttgccattttattttcttactggtgcgttttaataagcaagagtttttttttattttgatggagtctaatatatcatttattttcttttatatgtagtgctttttgtatccttgctaagataactttgcctactcccaaagttgggaagatattttctcatgttttcttttaaatgttctacagttttagcctttatatttagtttttttaattattattatactttaagttctagggtacatgtgcacaacgtgtaggtttgttacatatgtatacatgtgccatgttggtgtgctgcaccgattaactcgtcatttacattaggtatatctcctaatgctatccctcccccctccttccacctatgactggccctggtgtgtgatgttccccttcctgtgtccaagtgctcttatcgttcaattcccatctatgagtgagaacatgcagtgtttgattttttgtccttgtgatagtttgctgagaatgatggtttccagcttcatccatgtccctataaaggacatgaactcatccttttttatggctgcatagtattccatggtgtatatgtgccacattttcttaatccagtctatcattgatggacatttggcttggttccaagtctttgctattgtgaatagtgctgcaataatcgtacatgtgcatgtgtctttatagcagcatgatttatactcctttgggtatatacccagtaatgggatggctgggtcaaatagtatttctagctctggatccttgaggactcgccacactgtcttccacaatggttgaactagtttacagtcccaccaacagtgtaaaagtgttcctatttctccacattccctccagcaccttttgtttcctgactttttaatgatcaccattctaactggtgtgagatggtatgtcattgtggttttgatttgcatttctctgatggccattgatggctaatatccagaatctacaatgaactcaaacaaatttacaagaaaaaaacaaacaaccccatcaaaaagtgggcaaaggatatgaacagacacttctcaaaagaagacatttatgcagccaaaagacacatgaaaaaatgctcatcatcaatggccatcagagaaacgcaaatcaaattgtgtttatttgtttctcttgtcttatgcattggctaaaacctcctgtacaccactgaatagaaatggtgaaagtggatattcctgtcgtgtcctggtcttagggaaacaattcatgttcacaatttcagcactaaatatgatattaactataggcttttgtaaatgctctttatcagattgaggaagtgtctttctatttcttatttgctgtgagtttttaacatgaatagatgcattcatgttattaaattatgctttgaatgcattgattgattataaccaggttatttatgtcttctagtctgttaacatggcaaattatattgattaatttttgaatctttaacctgctttggtttcctgagatgtgccctactttataattatgtattaaaattagtgtgttagtattttcttgtgaaagtttgcttatacatttttgagggatatttgtctatcaacttcttttctctaatattttggccaggtttgggtaccaggattaagctagcttcaaaaaataggttgagaagggtcattcctcttccagtttctaaaataatttgtgtcagattgacactatttctttccttatacatttgatagaatttaccagaatataaccatcaagcatagagttttctttggggggaagtttattgataataagtttaatttctttgagagaaatataactgttgaaatattccatttctatgtgggtcagatttactaatttgtgtttataaaaacattttcattacatctaagttattatatacattaaaatagcatttaaaatttccttattatacttttaacatctgcatgttctatagtgatatctcctcttacattccagatattagtaatttatatattttgttttcttaaccactcttgttagggttcaccagccaaaattacctataaaaatccattacgttacccatcaagtatatgtgatattatgtatataaccctttatactatgttatcattttctttaacactttttttaatcaatattttttacagctcttatttcttacatatattcctatggaacatcaaaaaaagcaattactttttaatctaaacaaagtatttgtttttcagtgatcaattataaaaatatagaaatttcccataattttataaatatgtcttgactatttcaggttcaattgcatctaattctaagtaaatcatcactaagtatcatagcagcagaaagccataagattttaattcattatctctcattcctgaacatgcctccactcacccacccacatacctatgaacagagttaaagtcaaacatacatcaatgtgcatatgatactattccactgcatacaggaactcctacctgaatcaagacatatcccctttttattcctacagtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactataggtaagaagttgtatataaaagtatggttgtcacttttgggctacctgaaaacactgtgtctggacattctgtaggttaaaagtagacaaatagtggaaacaactggcaatagataatagctaattccctactgtaaatttttataataaatgaaaagcttgaaatttatactttcctgcagtgaaagaattctgaggatcttcaaacccaggtgtgaaagatagtgtttgtgcaaacctacatgaagtggctaactggagctgggcttcctgtcatccatcacaggtgtcctttccttccttatctgtcctttccttccttacctgtccttctcccaaattccttgtggtcttctccccaaatccccacaacattctgagtaagtttagctaacttatcaagttattttaaaaagcatatatgccttctctattagtcagagttttctacaaaaaaaaaagggaatcaataggaggatagatagatagatcattgataggagagatttctattaagaaattgacttttgtggttgtgggaactggcaatcgcaaaaatccataaggcaagccagtaggctagaaattcaggaaagagtgcagtattgagcctaaattccgcagggcaagaaactcaagcagattttctgtattgtactcttgagacagacttgcttcttcttcagggaacctctgtctttgctctagaggccttctactgatgaggtgatgcccaccacatcacggaaggcaatctactttactcaaagtttactgatttaaatgttaaccatgtcttaaaaatacttttagcattccctattcgctcccccttcaaccctcaaaaagaaaattaaaggtaagagagcaatactcattagagataagaaagagtaagaaacctagctcagctttgtctcagttttgtttcactaagatgataaaatagagaggtaaagcagaagttccatgtgtgaacaattaacttgtgaaaaggcaaatgtagtagaaaagagacattaggcagatggctgtgcatgttggccacacagaagcagcattggccatgaccagtgtgggtcctggttaggggaagagaactggctttgacaacaacagggtatctctgaggttataaaaagttgggttctgatcatttggagatgaggtccctatggatagggcaccatatctaaaggttcaccatttacattgcaaatatacattcagttctctgagagtgagcagagaaggcagaggttctcagtcttctgacaaggtcctggagcatcaggggagagcccattcttacaaaactccacaccagcatgcaagcccttacatgcacataagcactcacaacacaccaagagcctccaggtgacatctgccacctccaaatccccatatcccacatgctcaatgcacttgcagtctccatcccccagcagactgcaaatctgacatgcctcctccgaacggcaagggggagaggtacgtatggtacacacactgctgatggcataggcccctttggaaggggtagtgtgagtctcttggggctatggcaagcacccctggacaagcaggaagagaggtggtggaggcatgtctcacggtagcatctccttctaggtcctaatgggacacttcattaatggaactaccatttaagtgagtttaaactggatgcttctgattgagccccagagccagtgctccactgccaccacctgcaccctcacttccccttgtttaagcatcttccaacccagtaaggctgaagagggaagcatcctgccttcccacttctcttagcagagtagattgatatgattattcagattgtacaagaatctattccctctgaagtattgcttgatgaatgagcccctttttctaatttgctcaaagaaatcatttgagcttgaggaaaactgtccagagggcacgaggaccagccgttgtgatatgtaacaaggtagagaaacaaaagctaaatgaagaagagtgagcctcagaatcaaagaactggatttggatccctttaaaccattttacaggggcctgaatgtaattaacttctctgaaattcagtttccttatcaatatgctggtgataagtgactattgtttgaagacagcataagcaaagcatgcagtacttaggagatgtgttcttccttcaattcctctattattaaaagatgggcacagggcaggggcttcagctcagaaggccttgttgagaatggaatggagagcaggaacaagagagaggggcaaaggcattgccagcattctctgttcggctgttctccacccactgcctttcctcctgcttccctctaagtccagggcattttcccttttgataaacttccccttttacaacccatccaagggtgaaaaacaaagtcattacttttttttcagtacctctaaggcaaagcagcagaaacaggcagtcaccactacgaataagtgactacaacaagagctaggccaaactctgccatgtgggctgcattttattgggccggcaagtaactttaaatcccagctcacactctactgagtgaaagtctgatgaacccgcatcttcttgtgaacaactgcgcctgagatcagtcatgcaagaagtagcacccccacccccagacaactaacttcccaggctgtgaccaacaagcagccaagaggccaggacagggaagtctcaggacctttctaggaaatcaatacctttctctgggtttgttctgcctgaaataataccaatctccctccaacagcttagcatgtgtggagcatttgatactaacagcaaccctgcaaggcaggaaggcagtagggagaggcccaagaggaattcagcattaaggcagtgagactgacagaggggaccccctgaggacattctggaaggtcttagccagggccaggatgcagacccttcatgtcactgtagctgagacgaggtgcaaggttcacagcatataacctaattttattacaagaataaagactcagagtttaaatactcctgctttggggctcattagtaacaagttctccaatattcaaaaggcaaagtggatgtgttttagtgtaaaattaacactagctgctgtaacaaataagcccccaaacatatgatatctcaaacaccgtaggtttatttctcactcacatcagagtcaaaatggatgtttctaacctgcagctggggcttctcccagcagtattaggggcactttccatcttgtggctccaccgtctgtaatgcaggactccaagtggtggaagaggacggagcagaggagtcacacatgggtgtgtgtctggcccagggtggaagtggatgtgcatttcttctgcccacctcactcacaaggccacaccccactgcaagagaggctggagaatgcggactggatttaaacccaagaagaagaaatggttttctgaatagttggccatttactgacacaaaaagggtcaaagtgacttgcagaggagatgaattttaaatactataattatttccttggctgccctttagacagaatttatttctttttcttttccagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacggtatgtagttcaactacattaataaaataaaaacttatgaatgttttctattttgttggcctagtagtgcatttcccctgggagggcccaacaattttgctttcaaaatctaccttctactgaaagaatctcccaatattggccccatgaaaacctggatcttccctgatgcatactcttctagctctggttgttttcttctgctctaattttggtcttcagaatgtttctacattagtgagttggataacaatatagattgaggccaaattaatcctctgtattcaggggcctcaaaaagtgtcatgtctagtgccactttcataggcaaatcaggcaaaatgtatatctgcttatgatcaccaagtcgtagccacattctggcttatgagattcatgggaccagcatgaggtaaagaaaagaggcataatgtttgcctttgttttgtttttattttaaagcccaaggtctttgtttttgaagtaacagcttaatttttacccttcataatcaggagagttacttagatgctctcttcatgatttgttgaggttggaatgatttggcagtccctgaaatttattttggggaggaggtggcagaagagtggagtgtaccaggttatgagatttctcttaacccaccaacctaacttctgttctttctgcacctcagagatgaagaagagatgatgatttctcttcctcaagtccttcttattcttgctgtcctgttttttcaggccaagattggccttgtttgtttgcagtgtgatgcaagatgccacttgcataaatgtaacaactgccccaaaccacctgctccctccttctactcacccaccccacccttgatcctgccatctttcattattcatctgaaaattgcaccaattgaaaagcaacttagtggagaaaggaaggattatgaataaatgctgccaggacaattagttaactaaaaagaaaaatagataaattcaataaatacatgaatttttttgagatggagtcttgctcagtcatccaggttggagtgcaatggcgccatcttggctcactgcaacctccgcctcccgggttcaagcaattctcccatctcagcctcccaagtacctgtgattacaggcacccgccatcatgcccggctaatttttgtatttttgtagagctggggtttcaccatgttggccaggctggtcttgaactcctcacctcaggtgatctgcccacctcagcctcccaaagtgctgggattacaggcataagccaacacgccagccaaaaattgttttaattaaaaaaaattaaactaaatgcctagccaccttcatataacaacaacaaaataccagatgatttaaggaaattatataaaagtgaaactctaaacaaattagaaaaattatagccaaatgtttacataatcttgacatgaagaagaacattctaagcatcaaagctgtagaagaaaagaaaggattgagacatgcaactacataaaaagtggaggtttatatatgtcaacacacacaataatcaaaaatcaaaaatgcaaatttaaaagtaagcttaaattgccacataaacagctgatagatggttagtatcattaatagataaaggactcttataaatcattaaaaaaacaaatatcacaatagaaaaatgagcaaaaaaattgggaaaaatctcataaagtatggaatagataaattcaataaatatatgaaaatgaactaattatcaaataaatacagatataaatagcaatggacttctttttatctgtcaaattgatagagtggtttttttttaatcttaaagataatacactgtgtggtggagacttttgtctctttatcactattcacaatgtaaaatggcgtctttctggagagaaatgattcctgctcactaacctaacctaacctttcatctccccttaatatgtgaaaggatagagagaaaagaagaagatattgaagtgtggaaagggagatcctgggcagtgcctaactcacctgaataagacccatcatttcactctcctccttgaccactcacaacatcctttataagctcagattctgtccctaattttgctgttgactcctttacgtatcagagctccttattctaacaaatacgagacaacttcagagaatgcttatgggactaaaggaatcccaattgaaatgatttgggagatttaggcaacacctcttttcccatcctaagaatgtaactgcactctactctctagcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggtaaggaatggtggtagagtttttgttccctcagagtgctttgcatgtcaaagtgtgggagcaagtgagaggaagattgttgaaactaacctgcaaaataggacacccttggagggcactcttacactttctttggagaatgacttgcctgctgtctttgcgccttttgtgaagaacaaggaagcagagggagtggggtccttattagctgagaattagtacaagccatctgtattcctggaagctgccatacattttgaacaaaatccccacccactacgtccagttaaccaatttagcctgggaccccaatggctgctgtctctaaggcccctttaagaagcacctttattggtgtcaggtatgcaggcaagtgcggctgtcctatgtctccttttccagaaggatgaagatgtctttgggactggaactgagaatgtgtaggaactgagacatctcctccctaaaatttgcaacaggggtgaacatccctctcatcatctcctgctctggcttcttttccttggtagaaagtcaagaagggaagagagcattggtacctttgatgctagatcacgtttacatttcaagtggcagatgctctgggcctggtcacccaagtcaatgcccaagtagctgatgttcttcccactgtcaccgagatcagctcaactctttctctctatcaaagaactgtttctaagaaacaataagtgagacatgttattaagtaaaatcaaactaccctaaatatatacccacactttatgcttactgaatgctaaccgtgatctctccttatattccaggaggatgggatcctgtcttgccaagtgtcaccattctgagtttgttctctgtgtttttgttggtcatcttagcccatgtgctatggaaaaaaaggtgattttctttagtaaacaagagggttatttgtggagccccagaaaagcaggactcaggtaccatctagaaaagtttaaaataacagacttgacatttcaagaattatagtagcaaatatatgccctcctatttttagatcccagtcaatttaccatagttgatttcagaagaggcaaaaatatacaaactagacataagacaaacaatattgcacaaatattaatagtgtgtgtgtgtctgtacacatgtgcatctgtgcattgcatgtgtttgtgtatctgtgtctgtgtatatacacatgtgtgtgtgtgtgtgtgcgtgcgtgtgtgtatatgtgtgtgtgtgtgtgtgtatgtgtgtatgtgtgctgggtctgtctgtttgtttatctgtacatatatggttgtgttcatgtgtgtatacatgcaaatgtgtgctggcctacaggcagccaaactcaacacatattattttattcaattatcttccaccttgtctcttgatgcagggtcttccactaaacatgaaccttaccacccctgtcagattaattggtcagtaaactctgagtatctgtctgcctccaccactccacatatcatagagttacagatacatgttatcatacattgctatccaaactcagttcttcaaaattgtatgacaagaggtttatttattggattatttccctaggccatgacgcccccccccaaaaaaagaattctttaagaagtgttttccaaatattttttccacagcctcctaaaggacttagaggtgattacccatgctaacatggagtctatttgatctcatgactttctgcacacaaattcacatgatttttgtattttgctctgtggtagaaccatgccctgtgaattactcagtgttctaggaagttgccctcggcaattttgtattcctaggaccaaaagtgctctaatttgaaaaacgctaccataaaataattttcttgaatagcttagaacgtattcccaatttccactggaattaaagtaaaacctttacttccagtaaagacagtggataagatgacaataccaacagtgagcataaaagaccaggtctcattgtagctccatataaacaccaatactgcctccctgcaaacctcactcttcgctttagggtactttgcaacataattacattgtctcattcacaatagatcgttagcaaggagctctgttctatccactccttaaaccaagagcatgatactgtcagagaaaataaggtgtttgtccagtaacagaaatgttttcacaatctacctcaaataaggtgaaagagttgtttgtgtgccttctccttctgccgtttgtttcaagtatgaatgttctctggttttaggattaaacctgtcgtatggcctagtctccccgatcataagaaaactctggaacaactatgtaagaagccaaaaacggtaattgcttgaggtggggaaagaaacaccataatgttgaaatcttagtctaagaatgattaagactgacactcaacttacggtcttttatatatcacataaatgaaagtccttttaagactctgaagaataaagccaagatatgccacagggcagggggttggggaaaaatcaatatttacttcaaagttggagtatcacagctcagtcagaagtgaagccaactgtcattttttcacatcgtgtgtcaattttacaagaaagtttcgtaaacgttttagtttcctgaatcaaatgtatagcagcgcctctttgccacgcctctaacgcttctgcctttctctgcagagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatgaattataagaaaacccttccatcgacaaccaaatgatcactgagatggaaagtctggaatgcttgctctcccccgtagctcacagaagagaaagtcaacgtgaccttgctacacatcttcagcattctaagaaatcattttgctcttctagctcagaagcatttgcacaaagcaggaagaatctgttttccctgttgttggattagtcataagagtccatatgacccaattaaaattgcaaaactcagttaagtgaagaaagaaagatagacaaaagaagatagaaggatgtggtgaatgcaggaagaagaaaatgaaagatgtgagtggtgggtctatcattcaaattgactatttatccagcactataccactcttctcatttcttcctcacaataatattacaatgtgggcttatccattataacttttattttctttgtcatagatgctgaagttgaaagtagagattttaagtgatatccaaatttttctttcagctacagatgaggcacacattccaacttcaaccctctcttgccatgaacctgtcctattgttgagtgtcaaacatcaccactaagtggatggttatgtagtccattatccaaactgagtcgttttggaaagaaaaagttagacataattaacagtaagcataaactgtatatgtctaagagagatgtggatggatggtcattttacttaaagtggctatagggatgaacatgaaggacaaagtacatttatgggtgtggcataccatgaccatgtgtcaaaggaagtgggaaaaagaaaaaaaaagcaccaagatcatttgattttgttttgttgttttgtttgaaaacaaactcaagaagcaatgagttagaagccgagaagttccagagtcagttatcaagaccatgattttcctgctgctattatccattggcttctctgtgacattgtaggaggaactatggccaatctacaggagttcaacatttaacagtgaatggagtcctcctatgtgagtcctcctatgtgtggagacaccattaagaactaccccaagttctacatctctggatattgcctgaactacagaaaaagggggctgcgcacaccacaatgagtgccctacctgaaactatgctcacagaaacacaaagaagatgggtaagttattcaaattcaaatgttgatttatgactgcaagtcacaattttgaatccctgctgtgtataaccaatctcctgaagaaaacaacaaataactgaaagatactgtggttgggtgccttagcattaaaattctgtttaagtgttgacattgtttatttggattggagtgtctgtccggtcatgtattgtatccatgcattatattcagataaccacaacagctgctaatgcttgattatattctcagggactgcatgcaatgtaacattactggttggttctgccaattttcctcttggtatttataaaggaaaaccaaaactcttggtcagagacaatatgcaaaacagagatgtcaagtactatgtccaaatactgtgaaatataatgagaaataggtaacaaatttatcaatcaactatgtttggatccagggaatctcaagttattcaattcattctctgtaagcctttgtctctctcttcatccagacttttgccttcaaatacaagcatgcgctattttctggaa

SEQ ID NO: 66 - DNA, 21774 bp, Artificial Sequence
  Targeted Il7ra allele, without cassette (total 21774 bp)
  misc_feature (1)..(184): Mouse Sequence
  misc_feature (117)..(119): Start Codon
  misc_feature (185)..(310): Human Genomic Fragment 1
  misc_feature (311)..(387): Deleted Hyg Self-Deleting Cassette
  misc_feature (317)..(350): LoxP
  misc_feature (388)..(17493): Human Genomic Fragment 2
  misc_feature (17494)..(21774): Mouse Sequence
  misc_feature (20076)..(20078): Stop Codon
acagagctggtttgggtctccctctctctcattcacttgcacatacaagcgtgcttcttctctattctttctctctctctctctctctctctctctctctctctctctctctcagaatgatggctctgggtagagctttcgctatagttttctgcttaattcaagctgtttctggagaaagtggctatgctcaaaatggtgagtcatttctaagttttcttatggattttggattatctgtagcatggtttcaggttattcagttccctaacagacctgagtcaggcactgggtttgaatgcagtttgaggtcgagataacttcgtataatgtatgctatacgaagttatgctagtaactataacggtcctaaggtagcgagctagcaatttcccacatattcagtcattttttttaatgtttaaccaccatgacagggggcaggggatcaatactatgggtggtttataagacctcagtattctcaagaaggaatgcatttcactcccaagtgtagatcttaaatgttgaatgattactctgctcttacaaaaagaatgctcatgtagatgctatgactgtacttgtaggaaaatgtccaaagtaattttaccttgtcaggagatcaaactggattcattttgtttgactttttaagaaatcctgaaagcataactttcaggataaggtaatgtacagaagcaatagctttgtcttcagtgaccagtgctatatcctcagcacctaaatcagtggctagaatatagtagacatccaataacttttgaaagtgttttcaaaatactttagttttgagagatttatgtgagattttaagtaaataactgactagagaaagatctaaatgagtttactcattgaaatacactgaattgcctccacaccaacaaattggccatatgtaataattctttttgggatctaaaaaacttagtaccgagaagccaaccctgcccatacataaacacattgtaattataacaaaactaggcagaagcttctaacagcagcaggaggcatgtgggaatttagaccatcaacttgctcctgcaaattaagccctttctctttaagagttaaaaactatttggctatagacaatatcaaacacatcagcctaatgactcagcttatgcattttgagtcatgtaattacgaaggatggaaatccctagaattttctcattaagggaattgtcagagagtttgacattttttacagtatatgactcactttatgggggatgattattattctatgctaaactttgccttggatttccacaaagactgatgggaggcaggaaacataaatcttactctctttcatgtcatctatactcactagttcaccctggtgatcatactatttttaaaatatataagaatgctagttgaaagctgggttttcactccaactttttaagtttcagattttttagaagatgtataattaccctattcacatgattacgtcaaaatacttcccagtttggggtataggaattcacattcagttgctgcttgttgaaagttgtcaattttctgatcatcacaaggatgatcaagagaagaaagggatactttttaaaaatccaaatcatttacactattaatcaactaactccattcagtaggaagaagacttctagatgacactggcttgcctatgatacatattccacacaatttaaatttttatggataaatatgtctagatacctatttaaatatgaataatattaattattgagcatttaaagaataatagattaactcattattcaaaagctctatgtaatttcaaaaccatagtaattataacaccgtcaattgacataaactttttaaagagaagctcaaatgtttcatgtatattttcagaattagaattcttattttaccttttcattacttatttctcagaaaatattatactcatagctaatccctattaaatccttactgtgttctaagctacctctttgtaaatatccattcagtgattgctcatagcacgagtttacatattagaacacatgtcttagagaagttgcctacctgacagaggaccacaggtagagtatccagaatttaaacgcacatctgtccagctctaacaccacaggtcttaaccactgtgtacattaactactcttagccaagaatttttcagctcacgtcatgtagaatattctttttgtaaaatgccatcacattttataagtcattgaagggaatttttcttggttacaaagcaactctgccccataatatctactgaaaagccagtgagctgcttcctaaaacacagccattttaggtgcaggaaacagtgtataaatggctcattgtatattgtatgctttgccagactgagtggcagtgggagtcctttgttatgtgggtgctgacatctgctagagtgtgctgtctctattgaagaatcgtgaagacaaagccgacccacaggatgtctgaatccaaataataatacatgttctgtgtatagaattggtggaagagaaaatgtcaggacagtgtgaggactgccatgtaaggtcagaaccactgcatttagaaagctaccactgcacagggaagaaatctaagtctacaaaattagtgggctgtctctcattatttcgtgctgtcatcagaaggagggccataccctgctgaaactacataaagagcttttgctggtggcagaactgtgaactggatggattctgggaatggccagaaaaacaaatgcctgtggttgtgagcagtgcccacacccatggtctagctagggctgtttgagatttgttgctttgactgaaccaacctgtcattcaactggttggtccattcacagtcagctttattaactttcccattttccctactgagttatttaagtaaagaaagtgctattcggacagcccttggtctctgggacaatcaactgggatttgattttagtatattctgtctccagtgtaaagccttggaagcatctaatttctagtactgatgaaccaaaaatacatggaagcagtcctaggctcacacttgagcactctgagaatggctttgcttactccagattttctcaggtcccagtgggtgtatattttctgacatatttattccagcctcactttctatcatgtaaaacatacatacaaaatgtagatttcattatagggtctacaaaacagcttaagaaaccaaatactatgtgtgacagatcacactttccaaaagtaatagcaaaaaaaaaaaaaatctggttccccactttcttccagcatcctgctagaatctatcagatactgcgtctatagaagaatctataagaacagaagcagtatgtacaacattcacaggaagtttcaccaaatcggagtcctgccagatctaattttttttccctaatcacgtttgtctcagtcagtagcttaagacaatggaaataatcagtgccacttttaattgggatgcctttttaggcaagggaaagtgacctcttaaaaaagcaaaattctgactgcaagatagctatcattgtccttcatttaagacaaaaaaaatactagggagggaataaattatgatttgtaataaagtgaaaagtgagattaggtagcatggggataatggaaataaagtgtctcttctttgaaataatatgaacaatcaatgtaacaaatgtagcagaaaaaactccagtttaaatacagaaaagaatgtgttcaatgcctctggttctttaactcagaaatatttggaggttacttactcattatgatggattttttttttctattggaaaactctgttagcattgagcgtttttgttttttgttttttgttggttggttggttttgaagcatttttcttgtctttgcccttgggcttttcttccttgaatactacataatccattactatttcatgtctgccacagagtctgctattttattaaggtcatgccatatttcaaaaggatgcatttatttgtttcattaacagctgcatgtttgttcctccccaggagacttggaagatgcagaactggatgactactcattctcatgctatagccagttggaagtgaatggatcgcagcactcactgacctgtgcttttgaggacccagatgtcaacatcaccaatctggaatttgaaatatggtgagggatggtggttttaatggttgcttagacatcctctgtctctcttttcatatgctctttttaatagccacaaaagaaagaatatgtggcctaattaacaaatgttaacatctaaggaattcccaaaggcctcctgaaactccttgtccttcaccaaaaacactcatacaaatctcctctcacggttcagctttcagaccctgagactcagtcaaatgatgctctggatcttggggatcccacatccctcccaacttcatatcagaatttaaatcctgcgtctcctacaacacttctcaccaaaaatctgtttgcccaacacgagacaatccagtgtcttcaagttgcatctgagagttaaactgccttgtttccaatcccaataccagtgcttactagttttttgacctagagaaagttatgtaatgtatctatgcctcagtttcctcacctgtaaaatgagataacctgcctcacaggaaggctgtgatggttaaataatttcatcatataaatcattccaaatagtcggccagtgaataacgagtaatggggaagcaacattaaattataattctgtgaatattgacctaacttctaccatcttgacacaatttgacttcagatgatcctctcaatgtaaattttccaaaaatccacaggaataagttggcattttgtttcacaaggtctcacagaaaagacaaaggaaaagagtctggtttgaaagtttactaaaggtctcagggaactttatcttctccttctccttcatccataagtcatctcttgttgccaagggttactatctctggtgatttgagaaactactctagcttgaaattctgacctgaggctatctccaaattcatatccgaatgacctactttttagttagtgtcctagtgagcaaagtaaatcaagatccaccagtagtaatagaaggcttcctacattccatagacactgagacaattctccacagtctatagtccaaacaagccctgaattccagtttttgtcaatttatgggagcttcctgcatctatttatggagtgctttctgctgcagtccttagataaacatgctgttggacttgagtagtgtactgtgttctctgtctgcctctgttcacttccctaacacattttccaggaataaaatatgtcaaaagaacctgaaccagttcgatgtccacaatctaggctggaaatggattgcactaaaacagccataacaactcattcaaacaaggcactcattttcatgggcaaatcactctcccacacggaggtttgactttggcttctttaaccagctggctggtgggctgagtgttcatcctggtttctcttggccaagctgaggttgacctttctgttcactttcattcacaccatatttgaccacttccttgcccactcaaacatacttaccctttaacatatctcttgacttttcctgtcatattgtaatctgtccagagcctcctctatttgggttttccaattggattcagatatttcagttggaaagggactgccttaagaaagaaacgttttcagtggaaaatatatgtatgagctctttaatagatgaactcctggagttcagagcccttaaaaggatgcccagtttcacaagacagccatacggtcatccttgattgtccattgctcattaatttcattctcaaaatcatgggaatgagctgagaataccattttagatcctccttaaattcccaacagtaccagaaacttgctacaggttggggcctgtaattggatatttcacacatactttccttacaaatatattctatactcaagaattgaactaaaagttattgtcctagtttctccacatcccatgtttacctaaaattcagaaatgggaccccgctcccagtctccccttctatatttatttatcaaatcgtgacaacattaccatcttcagatctttccacctgatgtttgtcctaagcttattccctggtatctgtctagcttacccaaaaattcggtttttatttttatcctgttccaagttgggaaagcctatctaccccaacaaggaacacaactccctagtaactttgagacacacacacacatacacacctactctttaaagcctaaacaatcgcacactctaaaagatagcagttaacaaaagtaacgatttgggagaacagttttaaggaatgtccccaaaataatcaatacatttagccagttaattaacttaacatttcttcaccaatctctagttttcatgactgtaggagcttaaccagtcactctcagaccacaataaaccaaaggtgaaagattctgtaacaaaagctagggcactctcccctgcatttaacctcctggccagctcactcgaagccagacaaacaggttcctctttttgtgcagagtccaggaaccattctcgaaaggactcatttgagcacatgcagagaagagtgtacacacatccagttcaccaagggaagccaacacacattgtgggttgtaggtagtaaaaggccttcctagaacacactccttaggatttaaacaaaattacatcggttaatggaaagaattctttcatatacgcaaacttacccagaggaacttttcttctgcccagatcttcacttccaatttgacccagttatacctctttagagctatttggctgagcttaaacagcacataggaaaaacaaattggtaactgtgtttatcacagaagaggaaaattaaatttagggttgggaaaggaaaataaccctatgatattacttttattctacctttacaatgagaatatatacctttgttacttctttaatttttacattatttacttatttttctttgctttcttgtttgattacaatgcattttaggggtaaaatttatgtgtggtaaaatgcacaaaaattaagtgaatttggagaaatgtctatgacctgtagccattccaatggtaaagatatagaacttatttttcccctagaaggatgcttcatgttcctttccagtcaatcttcataccccaggagcaatcataattctcaattctattaccctttggtttttgccagtttctgatagttcttattaatagaatactctttattcttttctgtcttctttcatttaaccagtgtttgtgagagttagccatgttgatgtccatctcatagctcatcttttcaattgctaagtagtaattccactgtatgaatataccacaaatttttaattctttctcttcttgatgaacatttgtgttttttcaagtttgagactattattttttaggttgctgttcacattcttggacaaatcagtttgtgtatatatattttcatttttctggggtataaaacctcagaatggaattgctgtgtcataaggtaagcatgtatctaagtttataagaaaccgcccaacagtttttcaaagtggttataccattctactctccttccagcgatgcatgagagatatacatcatttgcaacgtttgactttgggatagtatctcgttaggtttttaattcgcatttgtcaaataacaaatgttgagcagcttttcatatacttggtcttttgcctgtcttctttgggctagtatctgttaaaagcactgagttatttgtccttttgttattgctggatatgagttctttatacattctgtatacatttcctttgtcagatagatgtattgcatctattttctattctgaagtttgccattttattttcttactggtgcgttttaataagcaagagtttttttttattttgatggagtctaatatatcatttattttcttttatatgtagtgctttttgtatccttgctaagataactttgcctactcccaaagttgggaagatattttctcatgttttcttttaaatgttctacagttttagcctttatatttagtttttttaattattattatactttaagttctagggtacatgtgcacaacgtgtaggtttgttacatatgtatacatgtgccatgttggtgtgctgcaccgattaactcgtcatttacattaggtatatctcctaatgctatccctcccccctccttccacctatgactggccctggtgtgtgatgttccccttcctgtgtccaagtgctcttatcgttcaattcccatctatgagtgagaacatgcagtgtttgattttttgtccttgtgatagtttgctgagaatgatggtttccagcttcatccatgtccctataaaggacatgaactcatccttttttatggctgcatagtattccatggtgtatatgtgccacattttcttaatccagtctatcattgatggacatttggcttggttccaagtctttgctattgtgaatagtgctgcaataatcgtacatgtgcatgtgtctttatagcagcatgatttatactcctttgggtatatacccagtaatgggatggctgggtcaaatagtatttctagctctggatccttgaggactcgccacactgtcttccacaatggttgaactagtttacagtcccaccaacagtgtaaaagtgttcctatttctccacattccctccagcaccttttgtttcctgactttttaatgatcaccattctaactggtgtgagatggtatgtcattgtggttttgatttgcatttctctgatggccattgatggctaatatccagaatctacaatgaactcaaacaaatttacaagaaaaaaacaaacaaccccatcaaaaagtgggcaaaggatatgaacagacacttctcaaaagaagacatttatgcagccaaaagacacatgaaaaaatgctcatcatcaatggccatcagagaaacgcaaatcaaattgtgtttatttgtttctcttgtcttatgcattggctaaaacctcctgtacaccactgaatagaaatggtgaaagtggatattcctgtcgtgtcctggtcttagggaaacaattcatgttcacaatttcagcactaaatatgatattaactataggcttttgtaaatgctctttatcagattgaggaagtgtctttctatttcttatttgctgtgagtttttaacatgaatagatgcattcatgttattaaattatgctttgaatgcattgattgattataaccaggttatttatgtcttctagtctgttaacatggcaaattatattgattaatttttgaatctttaacctgctttggtttcctgagatgtgccctactttataattatgtattaaaattagtgtgttagtattttcttgtgaaagtttgcttatacatttttgagggatatttgtctatcaacttcttttctctaatattttggccaggtttgggtaccaggattaagctagcttcaaaaaataggttgagaagggtcattcctcttccagtttctaaaataatttgtgtcagattgacactatttctttccttatacatttgatagaatttaccagaatataaccatcaagcatagagttttctttggggggaagtttattgataataagtttaatttctttgagagaaatataactgttgaaatattccatttctatgtgggtcagatttactaatttgtgtttataaaaacattttcattacatctaagttattatatacattaaaatagcatttaaaatttccttattatacttttaacatctgcatgttctatagtgatatctcctcttacattccagatattagtaatttatatattttgttttcttaaccactcttgttagggttcaccagccaaaattacctataaaaatccattacgttacccatcaagtatatgtgatattatgtatataaccctttatactatgttatcattttctttaacactttttttaatcaatattttttacagctcttatttcttacatatattcctatggaacatcaaaaaaagcaattactttttaatctaaacaaagtatttgtttttcagtgatcaattataaaaatatagaaatttcccataattttataaatatgtcttgactatttcaggttcaattgcatctaattctaagtaaatcatcactaagtatcatagcagcagaaagccataagattttaattcattatctctcattcctgaacatgcctccactcacccacccacatacctatgaacagagttaaagtcaaacatacatcaatgtgcatatgatactattccactgcatacaggaactcctacctgaatcaagacatatcccctttttattcctacagtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactataggtaagaagttgtatataaaagtatggttgtcacttttgggctacctgaaaacactgtgtctggacattctgtaggttaaaagtagacaaatagtggaaacaactggcaatagataatagctaattccctactgtaaatttttataataaatgaaaagcttgaaatttatactttcctgcagtgaaagaattctgaggatcttcaaacccaggtgtgaaagatagtgtttgtgcaaacctacatgaagtggctaactggagctgggcttcctgtcatccatcacaggtgtcctttccttccttatctgtcctttccttccttacctgtccttctcccaaattccttgtggtcttctccccaaatccccacaacattctgagtaagtttagctaacttatcaagttattttaaaaagcatatatgccttctctattagtcagagttttctacaaaaaaaaaagggaatcaataggaggatagatagatagatcattgataggagagatttctattaagaaattgacttttgtggttgtgggaactggcaatcgcaaaaatccataaggcaagccagtaggctagaaattcaggaaagagtgcagtattgagcctaaattccgcagggcaagaaactcaagcagattttctgtattgtactcttgagacagacttgcttcttcttcagggaacctctgtctttgctctagaggccttctactgatgaggtgatgcccaccacatcacggaaggcaatctactttactcaaagtttactgatttaaatgttaaccatgtcttaaaaatacttttagcattccctattcgctcccccttcaaccctcaaaaagaaaattaaaggtaagagagcaatactcattagagataagaaagagtaagaaacctagctcagctttgtctcagttttgtttcactaagatgataaaatagagaggtaaagcagaagttccatgtgtgaacaattaacttgtgaaaaggcaaatgtagtagaaaagagacattaggcagatggctgtgcatgttggccacacagaagcagcattggccatgaccagtgtgggtcctggttaggggaagagaactggctttgacaacaacagggtatctctgaggttataaaaagttgggttctgatcatttggagatgaggtccctatggatagggcaccatatctaaaggttcaccatttacattgcaaatatacattcagttctctgagagtgagcagagaaggcagaggttctcagtcttctgacaaggtcctggagcatcaggggagagcccattcttacaaaactccacaccagcatgcaagcccttacatgcacataagcactcacaacacaccaagagcctccaggtgacatctgccacctccaaatccccatatcccacatgctcaatgcacttgcagtctccatcccccagcagactgcaaatctgacatgcctcctccgaacggcaagggggagaggtacgtatggtacacacactgctgatggcataggcccctttggaaggggtagtgtgagtctcttggggctatggcaagcacccctggacaagcaggaagagaggtggtggaggcatgtctcacggtagcatctccttctaggtcctaatgggacacttcattaatggaactaccatttaagtgagtttaaactggatgcttctgattgagccccagagccagtgctccactgccaccacctgcaccctcacttccccttgtttaagcatcttccaacccagtaaggctgaagagggaagcatcctgccttcccacttctcttagcagagtagattgatatgattattcagattgtacaagaatctattccctctgaagtattgcttgatgaatgagcccctttttctaatttgctcaaagaaatcatttgagcttgaggaaaactgtccagagggcacgaggaccagccgttgtgatatgtaacaaggtagagaaacaaaagctaaatgaagaagagtgagcctcagaatcaaagaactggatttggatccctttaaaccattttacaggggcctgaatgtaattaacttctctgaaattcagtttccttatcaatatgctggtgataagtgactattgtttgaagacagcataagcaaagcatgcagtacttaggagatgtgttcttccttcaattcctctattattaaaagatgggcacagggcaggggcttcagctcagaaggccttgttgagaatggaatggagagcaggaacaagagagaggggcaaaggcattgccagcattctctgttcggctgttctccacccactgcctttcctcctgcttccctctaagtccagggcattttcccttttgataaacttccccttttacaacccatccaagggtgaaaaacaaagtcattacttttttttcagtacctctaaggcaaagcagcagaaacaggcagtcaccactacgaataagtgactacaacaagagctaggccaaactctgccatgtgggctgcattttattgggccggcaagtaactttaaatcccagctcacactctactgagtgaaagtctgatgaacccgcatcttcttgtgaacaactgcgcctgagatcagtcatgcaagaagtagcacccccacccccagacaactaacttcccaggctgtgaccaacaagcagccaagaggccaggacagggaagtctcaggacctttctaggaaatcaatacctttctctgggtttgttctgcctgaaataataccaatctccctccaacagcttagcatgtgtggagcatttgatactaacagcaaccctgcaaggcaggaaggcagtagggagaggcccaagaggaattcagcattaaggcagtgagactgacagaggggaccccctgaggacattctggaaggtcttagccagggccaggatgcagacccttcatgtcactgtagctgagacgaggtgcaaggttcacagcatataacctaattttattacaagaataaagactcagagtttaaatactcctgctttggggctcattagtaacaagttctccaatattcaaaaggcaaagtggatgtgttttagtgtaaaattaacactagctgctgtaacaaataagcccccaaacatatgatatctcaaacaccgtaggtttatttctcactcacatcagagtcaaaatggatgtttctaacctgcagctggggcttctcccagcagtattaggggcactttccatcttgtggctccaccgtctgtaatgcaggactccaagtggtggaagaggacggagcagaggagtcacacatgggtgtgtgtctggcccagggtggaagtggatgtgcatttcttctgcccacctcactcacaaggccacaccccactgcaagagaggctggagaatgcggactggatttaaacccaagaagaagaaatggttttctgaatagttggccatttactgacacaaaaagggtcaaagtgacttgcagaggagatgaattttaaatactataattatttccttggctgccctttagacagaatttatttctttttcttttccagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacggtatgtagttcaactacattaataaaataaaaacttatgaatgttttctattttgttggcctagtagtgcatttcccctgggagggcccaacaattttgctttcaaaatctaccttctactgaaagaatctcccaatattggccccatgaaaacctggatcttccctgatgcatactcttctagctctggttgttttcttctgctctaattttggtcttcagaatgtttctacattagtgagttggataacaatatagattgaggccaaattaatcctctgtattcaggggcctcaaaaagtgtcatgtctagtgccactttcataggcaaatcaggcaaaatgtatatctgcttatgatcaccaagtcgtagccacattctggcttatgagattcatgggaccagcatgaggtaaagaaaagaggcataatgtttgcctttgttttgtttttattttaaagcccaaggtctttgtttttgaagtaacagcttaatttttacccttcataatcaggagagttacttagatgctctcttcatgatttgttgaggttggaatgatttggcagtccctgaaatttattttggggaggaggtggcagaagagtggagtgtaccaggttatgagatttctcttaacccaccaacctaacttctgttctttctgcacctcagagatgaagaagagatgatgatttctcttcctcaagtccttcttattcttgctgtcctgttttttcaggccaagattggccttgtttgtttgcagtgtgatgcaagatgccacttgcataaatgtaacaactgccccaaaccacctgctccctccttctactcacccaccccacccttgatcctgccatctttcattattcatctgaaaattgcaccaattgaaaagcaacttagtggagaaaggaaggattatgaataaatgctgccaggacaattagttaactaaaaagaaaaatagataaattcaataaatacatgaatttttttgagatggagtcttgctcagtcatccaggttggagtgcaatggcgccatcttggctcactgcaacctccgcctcccgggttcaagcaattctcccatctcagcctcccaagtacctgtgattacaggcacccgccatcatgcccggctaatttttgtatttttgtagagctggggtttcaccatgttggccaggctggtcttgaactcctcacctcaggtgatctgcccacctcagcctcccaaagtgctgggattacaggcataagccaacacgccagccaaaaattgttttaattaaaaaaaattaaactaaatgcctagccaccttcatataacaacaacaaaataccagatgatttaaggaaattatataaaagtgaaactctaaacaaattagaaaaattatagccaaatgtttacataatcttgacatgaagaagaacattctaagcatcaaagctgtagaagaaaagaaaggattgagacatgcaactacataaaaagtggaggtttatatatgtcaacacacacaataatcaaaaatcaaaaatgcaaatttaaaagtaagcttaaattgccacataaacagctgatagatggttagtatcattaatagataaaggactcttataaatcattaaaaaaacaaatatcacaatagaaaaatgagcaaaaaaattgggaaaaatctcataaagtatggaatagataaattcaataaatatatgaaaatgaactaattatcaaataaatacagatataaatagcaatggacttctttttatctgtcaaattgatagagtggtttttttttaatcttaaagataatacactgtgtggtggagacttttgtctctttatcactattcacaatgtaaaatggcgtctttctggagagaaatgattcctgctcactaacctaacctaacctttcatctccccttaatatgtgaaaggatagagagaaaagaagaagatattgaagtgtggaaagggagatcctgggcagtgcctaactcacctgaataagacccatcatttcactctcctccttgaccactcacaacatcctttataagctcagattctgtccctaattttgctgttgactcctttacgtatcagagctccttattctaacaaatacgagacaacttcagagaatgcttatgggactaaaggaatcccaattgaaatgatttgggagatttaggcaacacctcttttcccatcctaagaatgtaactgcactctactctctagcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggtaaggaatggtggtagagtttttgttccctcagagtgctttgcatgtcaaagtgtgggagcaagtgagaggaagattgttgaaactaacctgcaaaataggacacccttggagggcactcttacactttctttggagaatgacttgcctgctgtctttgcgccttttgtgaagaacaaggaagcagagggagtggggtccttattagctgagaattagtacaagccatctgtattcctggaagctgccatacattttgaacaaaatccccacccactacgtccagttaaccaatttagcctgggaccccaatggctgctgtctctaaggcccctttaagaagcacctttattggtgtcaggtatgcaggcaagtgcggctgtcctatgtctccttttccagaaggatgaagatgtctttgggactggaactgagaatgtgtaggaactgagacatctcctccctaaaatttgcaacaggggtgaacatccctctcatcatctcctgctctggcttcttttccttggtagaaagtcaagaagggaagagagcattggtacctttgatgctagatcacgtttacatttcaagtggcagatgctctgggcctggtcacccaagtcaatgcccaagtagctgatgttcttcccactgtcaccgagatcagctcaactctttctctctatcaaagaactgtttctaagaaacaataagtgagacatgttattaagtaaaatcaaactaccctaaatatatacccacactttatgcttactgaatgctaaccgtgatctctccttatattccaggaggatgggatcctgtcttgccaagtgtcaccattctgagtttgttctctgtgtttttgttggtcatcttagcccatgtgctatggaaaaaaaggtgattttctttagtaaacaagagggttatttgtggagccccagaaaagcaggactcaggtaccatctagaaaagtttaaaataacagacttgacatttcaagaattatagtagcaaatatatgccctcctatttttagatcccagtcaatttaccatagttgatttcagaagaggcaaaaatatacaaactagacataagacaaacaatattgcacaaatattaatagtgtgtgtgtgtctgtacacatgtgcatctgtgcattgcatgtgtttgtgtatctgtgtctgtgtatatacacatgtgtgtgtgtgtgtgtgcgtgcgtgtgtgtatatgtgtgtgtgtgtgtgtgtatgtgtgtatgtgtgctgggtctgtctgtttgtttatctgtacatatatggttgtgttcatgtgtgtatacatgcaaatgtgtgctggcctacaggcagccaaactcaacacatattattttattcaattatcttccaccttgtctcttgatgcagggtcttccactaaacatgaaccttaccacccctgtcagattaattggtcagtaaactctgagtatctgtctgcctccaccactccacatatcatagagttacagatacatgttatcatacattgctatccaaactcagttcttcaaaattgtatgacaagaggtttatttattggattatttccctaggccatgacgcccccccccaaaaaaagaattctttaagaagtgttttccaaatattttttccacagcctcctaaaggacttagaggtgattacccatgctaacatggagtctatttgatctcatgactttctgcacacaaattcacatgatttttgtattttgctctgtggtagaaccatgccctgtgaattactcagtgttctaggaagttgccctcggcaattttgtattcctaggaccaaaagtgctctaatttgaaaaacgctaccataaaataattttcttgaatagcttagaacgtattcccaatttccactggaattaaagtaaaacctttacttccagtaaagacagtggataagatgacaataccaacagtgagcataaaagaccaggtctcattgtagctccatataaacaccaatactgcctccctgcaaacctcactcttcgctttagggtactttgcaacataattacattgtctcattcacaatagatcgttagcaaggagctctgttctatccactccttaaaccaagagcatgatactgtcagagaaaataaggtgtttgtccagtaacagaaatgttttcacaatctacctcaaataaggtgaaagagttgtttgtgtgccttctccttctgccgtttgtttcaagtatgaatgttctctggttttaggattaaacctgtcgtatggcctagtctccccgatcataagaaaactctggaacaactatgtaagaagccaaaaacggtaattgcttgaggtggggaaagaaacaccataatgttgaaatcttagtctaagaatgattaagactgacactcaacttacggtcttttatatatcacataaatgaaagtccttttaagactctgaagaataaagccaagatatgccacagggcagggggttggggaaaaatcaatatttacttcaaagttggagtatcacagctcagtcagaagtgaagccaactgtcattttttcacatcgtgtgtcaattttacaagaaagtttcgtaaacgttttagtttcctgaatcaaatgtatagcagcgcctctttgccacgcctctaacgcttctgcctttctctgcagagtctgaatgtgagtttcaatcccgaaagtttcctggactgccagattcatgaggtgaaaggcgttgaagccagggacgaggtggaaagttttctgcccaatgatcttcctgcacagccagaggagttggagacacagggacacagagccgctgtacacagtgcaaaccgctcgcctgagacttcagtcagcccaccagaaacagttagaagagagtcacccttaagatgcctggctagaaatctgagtacctgcaatgcccctccactcctttcctctaggtcccctgactacagagatggtgacagaaataggcctcctgtgtatcaagacttgctgccaaactctggaaacacaaatgtccctgtccctgtccctcaaccattgcctttccagtcgggaatcctgataccagtttctcagagacagcccatctccacttcctcagtactgaatcaagaagaagcgtatgtcaccatgtctagtttttaccaaaacaaatgaattataagaaaacccttccatcgacaaccaaatgatcactgagatggaaagtctggaatgcttgctctcccccgtagctcacagaagagaaagtcaacgtgaccttgctacacatcttcagcattctaagaaatcattttgctcttctagctcagaagcatttgcacaaagcaggaagaatctgttttccctgttgttggattagtcataagagtccatatgacccaattaaaattgcaaaactcagttaagtgaagaaagaaagatagacaaaagaagatagaaggatgtggtgaatgcaggaagaagaaaatgaaagatgtgagtggtgggtctatcattcaaattgactatttatccagcactataccactcttctcatttcttcctcacaataatattacaatgtgggcttatccattataacttttattttctttgtcatagatgctgaagttgaaagtagagattttaagtgatatccaaatttttctttcagctacagatgaggcacacattccaacttcaaccctctcttgccatgaacctgtcctattgttgagtgtcaaacatcaccactaagtggatggttatgtagtccattatccaaactgagtcgttttggaaagaaaaagttagacataattaacagtaagcataaactgtatatgtctaagagagatgtggatggatggtcattttacttaaagtggctatagggatgaacatgaaggacaaagtacatttatgggtgtggcataccatgaccatgtgtcaaaggaagtgggaaaaagaaaaaaaaagcaccaagatcatttgattttgttttgttgttttgtttgaaaacaaactcaagaagcaatgagttagaagccgagaagttccagagtcagttatcaagaccatgattttcctgctgctattatccattggcttctctgtgacattgtaggaggaactatggccaatctacaggagttcaacatttaacagtgaatggagtcctcctatgtgagtcctcctatgtgtggagacaccattaagaactaccccaagttctacatctctggatattgcctgaactacagaaaaagggggctgcgcacaccacaatgagtgccctacctgaaactatgctcacagaaacacaaagaagatgggtaagttattcaaattcaaatgttgatttatgactgcaagtcacaattttgaatccctgctgtgtataaccaatctcctgaagaaaacaacaaataactgaaagatactgtggttgggtgccttagcattaaaattctgtttaagtgttgacattgtttatttggattggagtgtctgtccggtcatgtattgtatccatgcattatattcagataaccacaacagctgctaatgcttgattatattctcagggactgcatgcaatgtaacattactggttggttctgccaattttcctcttggtatttataaaggaaaaccaaaactcttggtcagagacaatatgcaaaacagagatgtcaagtactatgtccaaatactgtgaaatataatgagaaataggtaacaaatttatcaatcaactatgtttggatccagggaatctcaagttattcaattcattctctgtaagcctttgtctctctcttcatccagacttttgccttcaaatacaagcatgcgctattttctggaa

SEQ ID NO: 67 - protein, 7 aa, Artificial Sequence
  synthetic peptide
AAAVTSR

SEQ ID NO: 68 - protein, 4 aa, Artificial Sequence
  synthetic peptide
QGGA

SEQ ID NO: 69 - DNA, 126 bp, Artificial Sequence
  synthetic oligonucleotide
ctatgctcaaaatggtgagtcatttctaagttttcttatggattttggattatctgtagcatggtttcaggttattcagttccctaacagacctgagtcaggcactgggtttgaatgcagtttgag

(19) United States
(12) Patent Application Publication
(10) Pub. No.: US 2022/0192165 A1
(43) Pub. Date: Jun. 23, 2022

(54) NON-HUMAN ANIMALS HAVING A HUMANIZED TSLP GENE, A HUMANIZED TSLP RECEPTOR GENE, AND/OR A HUMANIZED IL7RA GENE

(71) Applicant: Regeneron Pharmaceuticals, Inc., Tarrytown, NY (US)

(72) Inventors: Yajun Tang, White Plains, NY (US); Susannah Brydges, Scarsdale, NY (US); Subhashini Srivatsan, Wilton, CT (US); Davor Frleta, Forest Hills, NY (US); Cagan Gurer, Chappaqua, NY (US); Andrew J. Murphy, Croton-on-Hudson, NY (US)

(21) Appl. No.: 17/555,550

(22) Filed: Dec. 20, 2021

Related U.S. Application Data
(60) Provisional application No. 63/128,258, filed on Dec. 21, 2020.

(51) Int. Cl. A01K 67/027; A61K 49/00; C12N 15/85; G01N 33/50; C12N 15/89

(52) U.S. Cl. CPC A01K 67/0278; A61K 49/0008; C12N 15/8509; G01N 33/5088; C12N 15/89; G01N 2500/10; A01K 2217/052; A01K 2227/105; A01K 2267/0331; A01K 2267/0387; A01K 2207/15

(57) ABSTRACT

Disclosed herein are rodents (such as, but not limited to, mice and rats) genetically modified to comprise a humanized Tslp gene, a humanized Tslpr gene, a humanized 117ra gene, or a combination thereof. Compositions and methods for making such genetically modified rodents, as well as methods of using such genetically modified rodents as an animal model for diseases such as allergic diseases and cancer are provided.

### CROSS REFERENCE TO RELATED APPLICATION

This application claims the benefit of priority from U.S. Provisional Application No. 63/128,258, filed Dec. 21, 2020, the entire contents of which are incorporated herein by reference.

### INCORPORATION BY REFERENCE OF SEQUENCE LISTING

The Sequence Listing in the ASCII text file, named as 37301_10589US01_SequenceListing of 192 KB, created on Dec. 2, 2021 and submitted to the United States Patent and Trademark Office via EFS-Web, is incorporated herein by reference.

### BACKGROUND

Thymic stromal lymphopoietin (TSLP) acts through a heterodimer receptor composed of a chain specific for TSLP (referred to as “TSLPR” or “Tslpr”) and the IL7 receptor a chain, and is implicated in allergic diseases and certain cancer. Effective in vivo systems are desired for gaining a better understanding of pathogenesis of allergic diseases and cancer and for development of therapeutics.

### SUMMARY

In some embodiments, disclosed herein is a genetically modified rodent animal comprising a humanized Tslp gene in its genome, wherein the humanized Tslp gene comprises a rodent Tslp nucleic acid sequence and a human TSLP nucleic acid sequence, wherein the humanized Tslp gene encodes a humanized Tslp polypeptide comprising a mature protein sequence substantially identical to the mature protein sequence of a human TSLP protein.

In some embodiments, the humanized Tslp polypeptide comprises a mature protein sequence identical to the mature protein sequence of a human TSLP protein.

In some embodiments, the humanized Tslp protein comprises a signal peptide substantially identical to the signal peptide of a rodent Tslp protein. In some embodiments, the humanized Tslp protein comprises a signal peptide identical to the signal peptide of a rodent Tslp protein, e.g., an endogenous rodent Tslp protein.

In some embodiments, the human TSLP nucleic acid sequence in a humanized Tslp gene encodes at least a substantial portion of the mature protein sequence of a human TSLP protein. In some embodiments, the human TSLP nucleic acid sequence encodes the mature protein sequence of a human TSLP protein, e.g., amino acids 29-159 of a human TSLP protein (e.g., the human TSLP protein as set forth in SEQ ID NO: 3). In some embodiments, the human TSLP nucleic acid sequence comprises exon 1 from the codon encoding the first amino acid of the mature protein sequence, through the STOP codon in exon 4, of a human TSLP gene.

In some embodiments, the rodent Tslp nucleic acid sequence in a humanized Tslp gene comprises exonic sequences of a rodent Tslp gene (e.g., an endogenous rodent Tslp gene) that encode a rodent Tslp signal peptide. In some embodiments, the rodent animal is a mouse, and the rodent nucleic acid sequence in a humanized Tslp gene comprises exon 1, and a 5′ portion of exon 2 coding for signal peptide amino acids, of a mouse Tslp gene. In some embodiments, the rodent Tslp nucleic acid sequence in a humanized Tslp gene also comprises the 3′ UTR of a rodent Tslp gene (e.g., an endogenous rodent Tslp gene).

In some embodiments, the rodent animal is a mouse, and the humanized Tslp gene comprises (i) exon 1, and a 5′ portion of exon 2 coding for signal peptide amino acids, of a mouse Tslp gene, and (ii) exon 1 from the codon encoding the first amino acid of the mature protein sequence, through the STOP codon in exon 4, of a human TSLP gene. In some embodiments, the humanized Tslp gene further comprises the 3′ UTR of a mouse Tslp gene. In various embodiments, a mouse Tslp gene is an endogenous mouse Tslp gene.

In some embodiments, a humanized Tslp gene is operably linked to a rodent Tslp promoter, such as an endogenous rodent Tslp promoter.

In some embodiments, a humanized Tslp gene is located at a locus other than an endogenous rodent Tslp locus. In some embodiments, a humanized Tslp gene is located at an endogenous rodent Tslp locus.

In some of the embodiments where a humanized Tslp gene is located at an endogenous rodent Tslp locus, the humanized Tslp gene is formed as a result of replacement of a rodent Tslp genomic DNA at an endogenous rodent Tslp locus with a human TSLP nucleic acid. In some embodiments, the humanized Tslp gene is formed as a result of replacement of a rodent genomic DNA comprising exonic sequences encoding at least a substantial portion of the mature protein sequence of the endogenous rodent Tslp protein, with a human TSLP nucleic acid which encodes at least a substantial portion of the mature protein sequence of a human TSLP protein. In some embodiments, a humanized Tslp gene is formed as a result of replacement of a rodent genomic DNA comprising exonic sequences encoding the mature protein sequence of the endogenous rodent Tslp protein, with a human TSLP nucleic acid which encodes the mature protein sequence of a human TSLP protein. In some embodiments, the rodent animal is a mouse, and the mouse genomic DNA being replaced comprises exon 2 from the codon encoding the first amino acid of the mature mouse Tslp protein through the STOP codon in exon 5 of the endogenous mouse Tslp gene, and the human genomic DNA comprises exon 1 from the codon encoding the first amino acid of the mature human TSLP protein through the STOP codon in exon 4 of a human TSLP gene.

In some embodiments, the rodent animal is homozygous for a humanized Tslp gene. In some embodiments, the rodent animal is heterozygous for a humanized Tslp gene.

In some embodiments, a humanized Tslp polypeptide is expressed in a rodent animal from a humanized Tslp gene.

In some embodiments, a rodent animal further comprises in its genome, a humanized Tslpr gene, a humanized Il7ra gene, or a combination thereof.

In some embodiments, the rodent is a mouse or a rat.

In some embodiments, disclosed herein is an isolated rodent tissue or cell, whose genome comprises a humanized Tslp gene described herein. In some embodiments, the rodent cell is a rodent embryonic stem cell. In some embodiments, the rodent cell is an egg or a sperm. In some embodiments, an isolated rodent tissue or cell is a mouse tissue or cell, or a rat tissue or cell.

In some embodiments, disclosed herein is a rodent embryo comprising a rodent embryonic stem cell which comprises a humanized Tslp gene described herein.

In some embodiments, disclosed herein is a method of making a genetically modified rodent. In some embodiments, the method comprises modifying a rodent genome to comprise a humanized Tslp gene, wherein the humanized Tslp gene comprises a rodent Tslp nucleic acid sequence and a human TSLP nucleic acid sequence, and encodes a humanized Tslp polypeptide comprising a mature protein sequence substantially identical with the mature protein sequence of a human TSLP protein; and making a rodent comprising the modified rodent genome.

In some embodiments, modifying a rodent genome comprises the steps of introducing a nucleic acid molecule comprising a human TSLP nucleic acid sequence into the genome of a rodent embryonic stem (ES) cell, obtaining a rodent ES cell in which the human TSLP nucleic acid sequence has been integrated into an endogenous Tslp locus to replace a rodent Tslp genomic DNA thereby forming an humanized Tslp gene, and generating a rodent animal from the obtained rodent ES cell. In some embodiments, the human TSLP nucleic acid sequence encodes at least a substantial portion of the mature protein sequence of a human TSLP protein. In some embodiments, the nucleic acid molecule introduced into the ES cell further comprises a 5′ homology arm and a 3′ homology arm flanking the human TSLP nucleic acid sequence, and wherein the 5′ and 3′ homology arms are homologous to nucleic acid sequences at the endogenous rodent locus flanking the rodent Tslp genomic DNA to be replaced. In some embodiments, the humanized Tslp gene is operably linked to a rodent Tslp promoter, e.g., an endogenous rodent Tslp promoter at the endogenous rodent Tslp locus.

In some embodiments of the method, the rodent is a mouse or a rat.

In some embodiments, disclosed herein is a targeting nucleic acid construct, comprising a human TSLP nucleic acid sequence to be integrated into a rodent Tslp gene at an endogenous rodent Tslp locus, flanked by a 5′ nucleotide sequence and a 3′ nucleotide sequence that are homologous to nucleotide sequences at the rodent Tslp locus, wherein integration of the human TSLP nucleic acid sequence into the rodent Tslp gene results in a replacement of a rodent Tslp genomic DNA with the human TSLP nucleic acid sequence thereby forming a humanized Tslp gene, and wherein the human TSLP nucleic acid sequence encodes at least a substantial portion of the mature protein sequence of a human TSLP protein. In some embodiments of a targeting nucleic acid, the rodent is a mouse or a rat.

In some embodiments, disclosed herein is an in vitro method for generating a genetically modified rodent cell, comprising introducing into a rodent cell a targeting vector comprising a human TSLP nucleic sequence that encodes at least a substantial portion of the mature protein sequence of a human TSLP protein, flanked by rodent homology arms that mediate integration of the human TSLP nucleotide sequence into an endogenous rodent Tslp locus, which results in replacement of a rodent Tslp genomic DNA with the human TSLP nucleic acid sequence to form a humanized Tslp gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

In some embodiments, disclosed herein is a genetically modified rodent animal comprising a humanized Tslpr gene in its genome, wherein the humanized Tslpr gene comprises a rodent Tslpr nucleic acid sequence and a human TSLPR nucleic acid sequence, wherein the humanized Tslpr gene encodes a humanized Tslpr polypeptide comprising an ectodomain substantially identical to the ectodomain of a human TSLPR protein.

In some embodiments, the humanized Tslpr protein comprises a transmembrane-cytoplasmic sequence substantially identical to the transmembrane-cytoplasmic sequence of a rodent Tslpr protein (e.g., an endogenous rodent Tslpr protein). In some embodiments, the humanized Tslpr protein comprises a transmembrane-cytoplasmic sequence identical to the transmembrane-cytoplasmic sequence of a rodent Tslpr protein (e.g., an endogenous rodent Tslpr protein).

In some embodiments, the humanized Tslpr protein comprises a signal peptide substantially identical to the signal peptide of a rodent Tslpr protein. In some embodiments, the humanized Tslpr protein comprises a signal peptide identical to the signal peptide of a rodent Tslpr protein (e.g., an endogenous rodent Tslpr protein).

In some embodiments, the human TSLPR nucleic acid sequence in a humanized Tslpr gene encodes at least a substantial portion of the ectodomain of the human TSLPR protein. In some embodiments, the human TSLPR nucleic acid sequence in a humanized Tslpr gene encodes amino acids 29-231 of a human TSLPR (e.g., the human TSLPR as set forth in SEQ ID NO: 23). In some embodiments, the human TSLPR nucleic acid sequence comprises exon 2 through the codon encoding the last ectodomain amino acid in exon 6 of a human TSLPR gene.

In some embodiments, the rodent Tslpr nucleic acid sequence in a humanized Tslpr gene comprises exonic sequences of a rodent Tslpr gene that encode at least a substantial portion of the transmembrane-cytoplasmic sequence of a rodent Tslpr protein (e.g., an endogenous rodent Tslpr protein). In some embodiments, the rodent animal is a mouse, and the rodent Tslpr nucleic acid sequence comprises exon 6 from the codon encoding the first amino acid of the transmembrane domain through exon 8 of a mouse Tslpr gene (e.g., an endogenous mouse Tslpr gene).

In some embodiments, the rodent Tslpr nucleic acid sequence in a humanized Tslpr gene comprises exonic sequences of a rodent Tslpr gene that encode the signal peptide of a rodent Tslpr protein (e.g., an endogenous rodent Tslpr protein). In some embodiments, the rodent animal is a mouse, and the rodent nucleic acid sequence comprises exon 1 of a mouse Tslpr gene (e.g., an endogenous mouse Tslpr gene).

In some embodiments, the rodent animal is a mouse, and the humanized Tslpr gene comprises (i) exon 1 of a mouse Tslpr gene (e.g., an endogenous mouse Tslpr gene), (ii) exon 2 through the codon encoding the last amino acid of the ectodomain in exon 6 of a human TSLPR gene; and (iii) exon 6 from the codon encoding the first amino acid of the transmembrane domain through exon 8 of the mouse Tslpr gene.

In some embodiments, a humanized Tslpr gene is operably linked to a rodent Tslpr promoter, such as an endogenous rodent Tslpr promoter.

In some embodiments, a humanized Tslpr gene is located at a locus other than an endogenous rodent Tslpr locus. In some embodiments, a humanized Tslpr gene is located at an endogenous rodent Tslpr locus.

In some of the embodiments where a humanized Tslpr gene is located at an endogenous rodent Tslpr locus, the humanized Tslpr gene is formed as a result of replacement of a rodent Tslpr genomic DNA at an endogenous rodent Tslpr locus with a human TSLPR nucleic acid. In some embodiments, a humanized Tslpr gene is formed as a result of replacement of a rodent genomic DNA comprising exonic sequences encoding at least a substantial portion of the ectodomain of the endogenous rodent Tslpr protein, with the human TSLPR nucleic acid which encodes at least a substantial portion of the ectodomain of the human TSLPR protein. In some embodiments, the rodent animal is a mouse, and wherein the mouse genomic DNA being replaced comprises exon 2 through the codon encoding the last amino acid of the ectodomain in exon 6 of the endogenous mouse Tslpr gene, and the human genomic DNA comprises exon 2 through the codon encoding the last amino acid of the ectodomain in exon 6 of a human TSLPR gene.

In some embodiments, a rodent animal is heterozygous for a humanized Tslpr gene. In some embodiments, a rodent animal is homozygous for a humanized Tslpr gene.

In some embodiments, a humanized Tslpr polypeptide is expressed in a rodent animal from a humanized Tslpr gene.

In some embodiments, a rodent animal further comprises in its genome, a humanized Tslp gene, a humanized Il7ra gene, or a combination thereof.

In some embodiments, the rodent is a mouse or a rat.

In some embodiments, disclosed herein is an isolated rodent tissue or cell, whose genome comprises a humanized Tslpr gene described herein. In some embodiments, the rodent cell is a rodent embryonic stem cell. In some embodiments, the rodent cell is an egg or a sperm. In some embodiments, an isolated rodent tissue or cell is a mouse tissue or cell, or a rat tissue or cell.

In some embodiments, disclosed herein is a rodent embryo comprising a rodent embryonic stem cell which comprises a humanized Tslpr gene described herein.

In some embodiments, disclosed herein is a method of making a genetically modified rodent. In some embodiments, the method comprises modifying a rodent genome to comprise a humanized Tslpr gene, wherein the humanized Tslpr gene comprises a rodent Tslpr nucleic acid sequence and a human TSLPR nucleic acid sequence, and encodes a humanized Tslpr polypeptide comprising an ectodomain substantially identical with the ectodomain of a human TSLPR protein; and making a rodent comprising the modified rodent genome.

In some embodiments, modifying a rodent genome comprises the steps of introducing a nucleic acid molecule comprising a human TSLPR nucleic acid sequence into the genome of a rodent embryonic stem (ES) cell, obtaining a rodent ES cell in which the human TSLPR nucleic acid sequence has been integrated into an endogenous Tslpr locus to replace a rodent Tslpr genomic DNA thereby forming a humanized Tslpr gene, and generating a rodent animal from the obtained rodent ES cell. In some embodiments, the human TSLPR nucleic acid sequence encodes at least a substantial portion of the ectodomain of a human TSLPR protein. In some embodiments, the nucleic acid molecule introduced into the ES cell further comprises a 5′ homology arm and a 3′ homology arm flanking the human TSLPR nucleic acid sequence, and wherein the 5′ and 3′ homology arms are homologous to nucleic acid sequences at the endogenous rodent locus flanking the rodent Tslpr genomic DNA to be replaced. In some embodiments, the humanized Tslpr gene is operably linked to a rodent Tslpr promoter, e.g., an endogenous rodent Tslp promoter at the endogenous rodent Tslpr locus.

In some embodiments of the method, the rodent is a mouse or a rat.

In some embodiments, disclosed herein is a targeting nucleic acid construct, comprising a human TSLPR nucleic acid sequence to be integrated into a rodent Tslpr gene at an endogenous rodent Tslpr locus, flanked by a 5′ nucleotide sequence and a 3′ nucleotide sequence that are homologous to nucleotide sequences at the rodent Tslpr locus, wherein integration of the human TSLPR nucleic acid sequence into the rodent Tslpr gene results in a replacement of a rodent Tslpr genomic DNA with the human TSLPR nucleic acid sequence thereby forming a humanized Tslpr gene, and wherein the human TSLPR nucleic acid sequence encodes at least a substantial portion of the ectodomain of a human TSLPR protein. In some embodiments of a targeting nucleic acid, the rodent is a mouse or a rat.

In some embodiments, disclosed herein is an in vitro method for generating a genetically modified rodent cell, comprising introducing into a rodent cell a targeting vector comprising a human TSLPR nucleic sequence that encodes at least a substantial portion of the ectodomain of a human TSLPR protein, flanked by rodent homology arms that mediate integration of the human TSLPR nucleotide sequence into an endogenous rodent Tslpr locus, which results in replacement of a rodent Tslpr genomic DNA with the human TSLPR nucleic acid sequence to form a humanized Tslpr gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

In some embodiments, disclosed herein is a genetically modified rodent animal comprising a humanized Il7ra gene in its genome, wherein the humanized Il7ra gene comprises a rodent Il7ra nucleic acid sequence and a human IL7RA nucleic acid sequence, wherein the humanized Il7ra gene encodes a humanized Il7ra polypeptide comprising an ectodomain substantially identical to the ectodomain of a human IL7RA protein.

In some embodiments, the humanized Il7ra protein comprises a transmembrane-cytoplasmic sequence substantially identical to the transmembrane-cytoplasmic sequence of a rodent Il7ra protein (e.g., an endogenous rodent Il7ra protein). In some embodiments, the humanized Il7ra protein comprises a transmembrane-cytoplasmic sequence identical to the transmembrane-cytoplasmic sequence of a rodent Il7ra protein (e.g., an endogenous rodent Il7ra protein).

In some embodiments, the humanized Il7ra protein comprises a signal peptide substantially identical to the signal peptide of a rodent Il7ra protein. In some embodiments, the humanized Il7ra protein comprises a signal peptide identical to the signal peptide of a rodent Il7ra protein (e.g., an endogenous rodent Il7ra protein).

In some embodiments, the human IL7RA nucleic acid sequence in a humanized Il7ra gene encodes at least a substantial portion of the ectodomain of the human IL7RA protein. In some embodiments, the human IL7RA nucleic acid sequence in a humanized Il7ra gene encodes amino acids 21-236 of a human IL7RA protein (e.g., the human IL7RA protein as set forth in SEQ ID NO: 43). In some embodiments, the human IL7RA nucleic acid sequence comprises from the codon in exon 1 encoding the first amino acid of the mature protein through exon 5 of a human IL7RA gene.

In some embodiments, the rodent Il7ra nucleic acid sequence in a humanized Il7ra gene comprises sequences of a rodent Il7ra gene that encode at least a substantial portion of the transmembrane-cytoplasmic sequence of a rodent Il7ra protein (e.g., an endogenous rodent Il7ra protein). In some embodiments, the rodent animal is a mouse, and the rodent Il7ra nucleic acid sequence comprises exon 6 through exon 8 of a mouse Il7ra gene (e.g., an endogenous mouse Il7ra gene).

In some embodiments, the rodent Il7ra nucleic acid sequence in a humanized Il7ra gene comprises a portion of exon 1 of a rodent Il7ra gene that encode the signal peptide of a rodent Il7ra protein (e.g., an endogenous rodent Il7ra protein). In some embodiments, the rodent animal is a mouse, and the rodent nucleic acid sequence comprises a portion of exon 1 of a mouse Il7ra gene (e.g., an endogenous mouse Il7ra gene) that includes both the 5′ UTR and encodes the signal peptide of the mouse Il7ra.

In some embodiments, the rodent animal is a mouse, and the humanized Il7ra gene comprises (i) a portion of exon 1 of a mouse Il7ra gene (e.g., an endogenous mouse Il7ra gene) that encodes the signal peptide of the mouse Il7ra protein; (ii) exon 1 from the codon encoding the first amino acid of the mature protein through exon 5 of a human IL7RA gene; and (iii) exon 6 through exon 8 of the mouse Il7ra gene.

In some embodiments, a humanized Il7ra gene is operably linked to a rodent Il7ra promoter, such as an endogenous rodent Il7ra promoter.

In some embodiments, a humanized Il7ra gene is located at a locus other than an endogenous rodent Il7ra locus. In some embodiments, a humanized Il7ra gene is located at an endogenous rodent Il7ra locus.

In some of the embodiments where a humanized Il7ra gene is located at an endogenous rodent Il7ra locus, the humanized Il7ra gene is formed as a result of replacement of a rodent Il7ra genomic DNA at an endogenous rodent Il7ra locus with a human IL7RA nucleic acid. In some embodiments, a humanized Il7ra gene is formed as a result of replacement of a rodent genomic DNA comprising exonic sequences encoding at least a substantial portion of the ectodomain of the endogenous rodent Il7ra protein, with the human IL7RA nucleic acid which encodes at least a substantial portion of the ectodomain of the human IL7RA protein. In some embodiments, the rodent animal is a mouse, and wherein the mouse genomic DNA being replaced comprises from the codon in exon 1 encoding the first amino acid of the mature protein sequence through exon 5 of the endogenous mouse Il7ra gene, and the human genomic DNA comprises from the codon in exon 1 encoding the first amino acid of the mature protein sequence through exon 5 of a human IL7RA gene.

In some embodiments, a rodent animal is heterozygous for a humanized Il7ra gene. In some embodiments, a rodent animal is homozygous for a humanized Il7ra gene.

In some embodiments, a humanized Il7ra polypeptide is expressed in a rodent animal from a humanized Il7ra gene.

In some embodiments, a rodent animal further comprises in its genome, a humanized Tslp gene, a humanized Tslpr gene, or a combination thereof.

In some embodiments, the rodent is a mouse or a rat.

In some embodiments, disclosed herein is an isolated rodent tissue or cell, whose genome comprises a humanized Il7ra gene described herein. In some embodiments, the rodent cell is a rodent embryonic stem cell. In some embodiments, the rodent cell is an egg or a sperm. In some embodiments, an isolated rodent tissue or cell is a mouse tissue or cell, or a rat tissue or cell.

In some embodiments, disclosed herein is a rodent embryo comprising a rodent embryonic stem cell which comprises a humanized Il7ra gene described herein.

In some embodiments, disclosed herein is a method of making a genetically modified rodent. In some embodiments, the method comprises modifying a rodent genome to comprise a humanized Il7ra gene, wherein the humanized Il7ra gene comprises a rodent Il7ra nucleic acid sequence and a human IL7RA nucleic acid sequence, and encodes a humanized Il7ra polypeptide comprising an ectodomain substantially identical with the ectodomain of a human IL7RA protein; and making a rodent comprising the modified rodent genome.

In some embodiments, modifying a rodent genome comprises the steps of introducing a nucleic acid molecule comprising a human IL7RA nucleic acid sequence into the genome of a rodent embryonic stem (ES) cell, obtaining a rodent ES cell in which the human IL7RA nucleic acid sequence has been integrated into an endogenous Il7ra locus to replace a rodent Il7ra genomic DNA thereby forming a humanized Il7ra gene, and generating a rodent animal from the obtained rodent ES cell. In some embodiments, the human IL7RA nucleic acid sequence encodes at least a substantial portion of the ectodomain of a human IL7RA protein. In some embodiments, the nucleic acid molecule introduced into the ES cell further comprises a 5′ homology arm and a 3′ homology arm flanking the human IL7RA nucleic acid sequence, and wherein the 5′ and 3′ homology arms are homologous to nucleic acid sequences at the endogenous rodent locus flanking the rodent Il7ra genomic DNA to be replaced. In some embodiments, the humanized Il7ra gene is operably linked to a rodent Il7ra promoter, e.g., an endogenous rodent Il7ra promoter at the endogenous rodent Il7ra locus.

In some embodiments of the method, the rodent is a mouse or a rat.

In some embodiments, disclosed herein is a targeting nucleic acid construct, comprising a human IL7RA nucleic acid sequence to be integrated into a rodent Il7ra gene at an endogenous rodent Il7ra locus, flanked by a 5′ nucleotide sequence and a 3′ nucleotide sequence that are homologous to nucleotide sequences at the rodent Il7ra locus, wherein integration of the human IL7RA nucleic acid sequence into the rodent Il7ra gene results in a replacement of a rodent Il7ra genomic DNA with the human IL7RA nucleic acid sequence thereby forming a humanized Il7ra gene, and wherein the human IL7RA nucleic acid sequence encodes at least a substantial portion of the ectodomain of a human IL7RA protein. In some embodiments of a targeting nucleic acid, the rodent is a mouse or a rat.

In some embodiments, disclosed herein is an in vitro method for generating a genetically modified rodent cell, comprising introducing into a rodent cell a targeting vector comprising a human IL7RA nucleic sequence that encodes at least a substantial portion of the ectodomain of a human IL7RA protein, flanked by rodent homology arms that mediate integration of the human IL7RA nucleotide sequence into an endogenous rodent Il7ra locus, which results in replacement of a rodent Il7ra genomic DNA with the human IL7RA nucleic acid sequence to form a humanized Il7ra gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

In some embodiments, rodents disclosed herein comprise one or more additional genetic modifications in their genome such as a humanized Sirpα gene, a disruption in an endogenous RAG2 gene, a disruption in an endogenous IL-2RG gene, a humanized Tpo gene, and a humanized GM-CSF/IL-3 locus. A rodent can be heterozygous or homozygous for any of such additional genetic modifications.

In some embodiments, rodents disclosed herein comprise in their genome a humanized Tslp gene and a humanized Sirpα gene, and are homozygous null for both RAG2 and IL-2RG genes. In some such embodiments, a rodent further comprises in its genome a humanized Tpo gene and/or a humanized GM-CSF/IL-3 locus. A rodent can be heterozygous or homozygous for a humanized gene.

In some embodiments, rodents disclosed herein comprise in their genome a humanized Tslp gene, a humanized Tslpr gene, and a humanized Sirpα gene, and are homozygous null for both RAG2 and IL-2RG genes. In some such embodiments, a rodent further comprises in its genome a humanized Tpo gene and/or a humanized GM-CSF/IL-3 locus. Rodents can be homozygous or heterozygous for a humanized gene.

In some embodiments, rodents disclosed herein comprise in their genome a humanized Tslp gene, a humanized Tslpr gene, a humanized Il7ra gene, and a humanized Sirpα gene, and are homozygous null for both RAG2 and IL-2RG genes. In some such embodiments, a rodent further comprises in its genome a humanized Tpo gene and/or a humanized GM-CSF/IL-3 locus. Rodents can be homozygous or heterozygous for a humanized gene.

In some embodiments, a genetically modified rodent animal comprising a humanized Tslp gene, a humanized Tslpr gene, a humanized Il7ra gene, or a combination thereof, optionally with one or more additional genetic modifications, as disclosed herein, is used in the preparation of a rodent animal model of allergic diseases (e.g., airway or skin inflammation) or cancer.

In some embodiments, disclosed herein is a method of testing a candidate agent for treating an allergic condition, the method comprising inducing an allergic condition in a genetically modified rodent animal disclosed herein, administering a candidate agent to the rodent animal; and determining whether the candidate agent inhibits the allergic condition in the rodent animal.

In some embodiments, disclosed herein is a method of testing a candidate agent for treating cancer, the method comprising engrafting human cancer cells in a genetically modified rodent animal disclosed herein, administering a candidate agent to the rodent animal; and determining whether the candidate agent inhibits the growth of the cancer cells in the rodent animal. In some embodiments, the cancer is a Th2 driven cancer, including e.g., breast cancer, lung cancer, and pancreatic cancer.

### BRIEF DESCRIPTION OF THE DRAWINGS

The file of this patent or application contains at least one drawing executed in color. Copies of this patent with color drawing(s) will be provided by the Patent and Trademark Office upon request and payment of the necessary fee.

The accompanying drawings, which are incorporated in and constitute a part of this specification, illustrate several embodiments and together with the description illustrate the disclosed compositions and methods.

FIG. 1A depicts an exemplary embodiment of a strategy for humanization of a mouse Tslp locus. The mouse Tslp gene and the human TSLP gene are represented by horizontal lines, with their exons being represented by boxes placed above the lines. A contiguous mouse Tslp genomic fragment of 3486 bp at an endogenous mouse Tslp locus, which begins from the codon in exon 2 encoding amino acid 20 through the STOP codon in exon 5, is replaced by a human TSLP genomic fragment of 4100 bp that begins from the codon in human TSLP exon 1 encoding amino acid 29 through the STOP codon in exon 4. The replacement results in a mouse-human hybrid (“humanized”) Tslp protein that includes the mouse Tslp signal peptide and the human mature TSLP polypeptide. See also FIGS. 1D and 1F.

FIG. 1B depicts an exemplary embodiment of a strategy for humanization of a mouse TSLP locus, not to scale, of a nucleic acid comprising a mouse Tslp locus, and a targeting nucleic acid construct comprising a humanized mouse Tslp locus wherein a mouse genomic fragment of 3.49 kB at a mouse Tslp locus has been replaced with a humanization fragment comprising a Human Genomic Fragment 1 of 1.9 kb (from the codon in exon 1 encoding amino acid 29 through 257 bp after the 3′ end of exon 3 of human TLSP), a Floxed HUb-Puro cassette of 4.4 kb (inserted in human TLSP intron 3), and a Human Genomic Fragment 2 of 2.2 kb (from 258 bp after the end of exon 3 through the STOP codon in exon 4 of human TLSP). The humanization fragment in the targeting construct is flanked by a mouse 5′ homology arm of 114.3 kb and a mouse 3′ homology arm of 65.3 kb. The targeting nucleic acid construct comprising the humanized mouse Tslp locus can be introduced into a mouse embryonic stem cell for targeted insertion into the mouse Tslp gene in the mouse genome. The locations of primers and probes used in human gain of allele and mouse loss of allele assays to confirm correct targeting are also indicated. The sequences of the primers and probes are set forth in Table 6.

FIG. 1C depicts an exemplary embodiment of a strategy for humanization of a mouse TSLP allele, not to scale, of the humanized Tslp allele designated as MAID #7466 resulting from the replacement of the mouse Tslp genomic fragment described above for FIG. 1B, with the humanization fragment which comprises the Human Genomic Fragment 1, the Floxed HUb-Puro cassette, and the Human Genomic Fragment 2. After the Floxed HUb-Puro cassette has been removed, the humanized Tslp allele is designated as MAID #7467.

FIG. 1D shows exemplary embodiments of the protein sequences of mouse Tslp (SEQ ID NO: 1), human TSLP isoform 1 (SEQ ID NO: 3) (in bold italics), and humanized (hybrid) Tslp (SEQ ID NO: 5) (with the human portion in bold italics). The signal peptide is underlined in each protein sequence.

FIG. 1E shows exemplary embodiments of the mRNA sequences of mouse Tslp (SEQ ID NO: 2), human TSLP isoform 1 (SEQ ID NO: 4) (in bold italics), and humanized (hybrid) Tslp (SEQ ID NO: 6) (with the human portion in bold italics). The portion encoding the signal peptide is underlined in each mRNA.

FIG. 1F shows alignment of exemplary embodiments of mouse Tslp (“mTslp”, SEQ ID NO: 1), human TSLP isoform 1 (“hTSLP”, SEQ ID NO: 3), and humanized (hybrid) Tslp (SEQ ID NO: 5) protein sequences. The signal peptides of the proteins are boxed. The junctions between the mouse and human sequences in forming the humanized (hybrid) Tslp protein are indicated by the arrows at the 5′ (N-terminal) and the 3′ (C-terminal) of the molecules. The triangles represent the location of human intron 3 (2429 bp), into which the Floxed HUb-Puro cassette is inserted.

FIG. 1G shows, in exemplary embodiments, that mice heterozygous for Tslp humanization as described n Example 1 expressed mature human TSLP protein in mouse serum (middle), with mice without Tslp humanization as a negative control (left) and normal human serum as a positive control (right). Each dot represents one mouse.

FIG. 2A depicts an exemplary embodiment of a strategy for humanization of a mouse Tslpr locus. The mouse Tslpr gene and the human TSLPR gene are represented by horizontal lines, with their exons being represented by boxes placed above the lines. A contiguous mouse Tslpr genomic fragment of 2362 bp at an endogenous mouse Tslpr locus, which begins from intron 1 at 328 bp before exon 2 and ends at the 47th bp in exon 6, is replaced by a human TSLPR genomic fragment of 13743 bp that begins from intron 1 at 909 bp before exon 2 and ends at the 47th bp in exon 6. The replacement results in a deletion of amino acids 27 to 243 of mouse Tslpr, but preserves the mouse Tslpr signal peptide (amino acid 1-19), the first 7 amino acids of the mouse Tslpr mature protein, and the mouse Tslpr transmembrane domain (amino acids 244-264) and intracellular domain, and inserts a substantial portion of the human TSLPR ectodomain (beginning at amino acid 27, and ending at amino acid 231, just before the human transmembrane domain of amino acids 232-252). See also FIG. 2D.

FIG. 2B depicts an exemplary embodiment of a strategy for humanization of a mouse Tslpr locus, not to scale, of a nucleic acid comprising a mouse Tslpr locus, and a targeting nucleic acid construct comprising a humanized mouse Tslpr locus wherein a mouse genomic fragment of 2.36 kB at a mouse Tslpr locus has been replaced with a humanization fragment comprising Floxed HUb-Neo cassette of 4.8 kb and a human genomic fragment of 13.7 kb (comprising a 3′ portion of intron 1, exon 2 through the 47th bp in exon 6 of human TSLPR). The humanization fragment in the targeting construct is flanked by a mouse 5′ homology arm of 29.1 kb (up to 328 bp before exon 2 of mouse Tslpr) and a mouse 3′ homology arm of 133.2 kb (from the 48^{th} bp in exon 6 through exon 8 of mouse Tslpr, followed by mouse 3′ genomic sequence). The targeting nucleic acid construct comprising the humanized mouse Tslpr locus can be introduced into a mouse embryonic stem cell for targeted insertion into the mouse genome. The locations of primers and probes used in human gain of allele and mouse loss of allele assays to confirm correct targeting are also indicated. The sequences of the primers and probes are set forth in Table 9.

FIG. 2C depicts an exemplary embodiment of a strategy for humanization of a mouse TSLP allele, not to scale, of the humanized Tslpr allele designated as MAID #7558 resulting from the replacement of the mouse Tslpr genomic fragment described above for FIG. 2B, with the humanization fragment which comprises the Floxed HUb-Neo cassette of 4.8 kb and the human TSLPR genomic fragment of 13743 bp. After the Floxed HUb-Neo cassette has been removed, the humanized Tslpr allele is designated as MAID #7559.

FIG. 2D shows exemplary embodiments of the protein sequences of mouse Tslpr (SEQ ID NO: 21), human TSLPR (SEQ ID NO: 23) (in bold italics), and humanized (hybrid) Tslpr (SEQ ID NO: 25) (with the human portion in bold italics). The signal peptide (“SP”) and transmembrane segment (“TM”) are underlined in each protein sequence.

FIG. 2E shows exemplary embodiments of the mRNA sequences of mouse Tslpr (SEQ ID NO: 22), human TSLPR (SEQ ID NO: 24) (in bold italics), and humanized (hybrid) Tslpr (SEQ ID NO: 26) (with the human portion in bold italics). The portions encoding the signal peptide and the transmembrane segment, respectively, are underlined in each mRNA.

FIG. 2F shows alignment of exemplary embodiments of mouse Tslpr (“mTslpr”, SEQ ID NO: 21), human TSLPR (“hTSLPR”, SEQ ID NO: 23), and humanized (hybrid) Tslpr (SEQ ID NO: 25) protein sequences. The signal peptides of the proteins are boxed with dashed lines. The transmembrane domains are boxed with solid lines. The junctions between the mouse and human sequences in forming the humanized (hybrid) Tslpr are indicated by triangles at the 5′ (in intron 1) and the 3′ (in exon 6) of the molecules.

FIG. 3A depicts an exemplary embodiment of a strategy for humanization of a mouse Il7ra locus. The mouse Il7ra gene and the human IL7RA gene are represented by horizontal lines, with their exons being represented by boxes placed above the lines. A contiguous mouse Il7ra genomic fragment of 19235 bp at an endogenous mouse Il7ra locus, which begins from the 69^{th} bp in the coding sequence of exon 1 through a 5′ portion of intron 5, is replaced by a human IL7RA genomic fragment of 17232 bp that includes from the 69^{th} bp in the coding sequence of exon 1 through a 5′ portion of intron 5. The replacement results in a mouse-human hybrid (“humanized”) Tslp protein that includes the mouse IL7ra signal peptide, an ectodomain that is substantially human (except for the last two amino acids, Gly-Trp), and the transmembrane and intracellular domains of mouse Il7ra. See also FIG. 3D.

FIG. 3B depicts an exemplary embodiment of a strategy for humanization of a mouse IL7ra locus, not to scale, of a nucleic acid comprising a mouse IL7ra locus, and a targeting nucleic acid construct comprising a humanized mouse IL7ra locus wherein a mouse genomic fragment of 19.2 kB at a mouse IL7ra locus has been replaced with a humanization fragment comprising a Human Genomic Fragment 1 of 126 bp (including the last 14 bp in exon 1 and the first 112 bp in intron 1 of human IL7RA), a Floxed HUb-Hyg cassette of 5.2 kb (inserted in human IL7RA intron 1), and a Human Genomic Fragment 2 of 17106 bp (including a 3′ portion of intron 1, exon 2 through exon 5, and a 5′ portion of intron 5 of human IL7RA). The humanization fragment in the targeting construct is flanked by a mouse 5′ homology arm of 48.8 kb and a mouse 3′ homology arm of 124.3 kb. The targeting nucleic acid construct comprising the humanized mouse Il7ra locus can be introduced into a mouse embryonic stem cell for targeted insertion into the mouse genome. The locations of primers and probes used in human gain of allele and mouse loss of allele assays to confirm correct targeting are also indicated. The sequences of the primers and probes are set forth in Table 12.

FIG. 3C depicts an exemplary embodiment of a strategy for humanization of a mouse Il7ra allele, not to scale, of the humanized Il7ra allele designated as MAID #7266 resulting from the replacement of the mouse IL7ra genomic fragment, described above for FIG. 3B, with the humanization fragment which comprises the Human Genomic Fragment 1 (126 bp), the Floxed HUb-Hyg cassette, and the Human Genomic Fragment 2 (17106 bp). After the Floxed HUb-hyg cassette has been removed, the humanized Il7ra allele is designated as MAID #7267.

FIG. 3D shows the protein sequences of mouse Il7ra (SEQ ID NO: 41), human IL7RA (SEQ ID NO: 43) (in bold italics), and humanized (hybrid) Il7ra (SEQ ID NO: 45) (with the human portion in bold italics). The signal peptide and transmembrane domain are underlined in each protein sequence.

FIG. 3E shows exemplary embodiments of the coding sequences (CDS) of mouse Il7ra (SEQ ID NO: 42), human IL7RA (SEQ ID NO: 44) (in bold italics), and humanized (hybrid) Il7ra (SEQ ID NO: 46) (with the human portion in bold italics). The portion of the human IL7RA sequence that is used in the humanization is underlined. The portion of the hybrid Il7ra sequence having the human origin is also underlined.

FIG. 3F shows alignment of exemplary embodiments of mouse Il7ra (SEQ ID NO: 41, top) and human IL7RA (SEQ ID NO: 43, bottom) protein sequences. The signal peptide and the transmembrane segment (in a box) of the proteins are indicated. The junctions between the mouse and human sequences in forming a humanized (hybrid) Il7ra described in FIGS. 3A-3E are indicated by a vertical line at the N-terminal (the “5′ junction”) and a line near the C-terminus of the ectodomain (the “3′ junction”). The amino acids of the ectodomain involved in the humanization are highlighted (beginning from the amino acid immediately after the 5′ junction and ending at the 3′ junction). The triangles represent junctions of exons in the coding sequences.

FIG. 4A-4E. Mice were sensitized i.p. to either saline and alum (alum only) or ovalbumin and alum (Ova-alum) on day 0 and day 14, followed by 4 consecutive intranasal challenges with Ova on day 21 through 24. On day 25, lung tissue and serum were collected for further analysis. 4A. Assessment of lung cellular infiltrates by flow cytometry. Cell frequencies of lung tissue eosinophils are plotted as frequency of total live cells. 4B. Assessment of Muc5ac mRNA expression levels measured by real-time qPCR and expressed relative to β2m (β2-microglobulin) control mRNA expression. ELISA analysis of serum Ova-specific IgE (4C) and Ova-specific IgG1 (4D). Each point represents a single mouse. Symbols represent statistical significance compared to saline control (*). 4E depicts the experimental scheme: Mice were sensitized i.p. to either saline and alum (alum only) or ovalbumin and alum (Ova-alum) on day 0 and day 14, followed by 4 consecutive intranasal challenges with Ova on day 21 through 24. On day 25, lung tissue and serum were collected for further analysis of the parameters shown in 4A-4D.

### DETAILED DESCRIPTION

Disclosed herein are rodents (such as, but not limited to, mice and rats) genetically modified to comprise a humanized Tslp gene, a humanized Tslpr gene, a humanized Il7ra gene, or a combination thereof. The rodents disclosed herein can be used as, e.g., but not limited to, a model for Th2-driven allergic diseases, or as a model for inflammatory Th2-driven cancer. Compositions and methods for making such genetically modified rodents, as well as methods of using such genetically modified rodents for testing candidate therapeutic agents for the treatment of allergy or cancer are provided and further described below.

### Tslp Humanized Rodents

Thymic stromal lymphopoietic (TSLP) is a member of the 4-helix bundle cytokine family and a distant paralog of interleukin-7 (IL-7). TSLP was initially discovered in the culture supernant of a mouse thymic stromal cell line, and has been shown to act as a growth factor for T cells and B cells. See, e.g., Tsilingiri et al., Cell Mol. Gastroenterology & Hepatology 2017; 3: 174-182, which is hereby incorporated by reference in its entirety. TSLP expressing cells include epithelial cells, keratinocytes, fibroblasts, stromal cells, dendritic cells, mast cells and basophils. See, Tsilingiri et al. (2017), supra. Two TSLP isoforms exist in human: the long TSLP isoform (isoform 1) which is expressed at low/undetectable level at steady state, and upregulated during inflammation in several tissues and is a hallmark of exacerbated Th2 responses in multiple Th2-related diseases (e.g., but not limited to, atopic dermatitis, asthma, allergic responses, and certain types of cancer); and the short TSLP isoform (isoform 2) which is constitutively expressed from a separate promoter and mediates certain immune homeostatic functions in the gut and the thymus. See, Tsilingiri et al. (2017), supra. Unless specifically indicated, the exon numberings of a human TSLP gene are based on exons encoding the long isoform (human TSLP protein isoform 1).

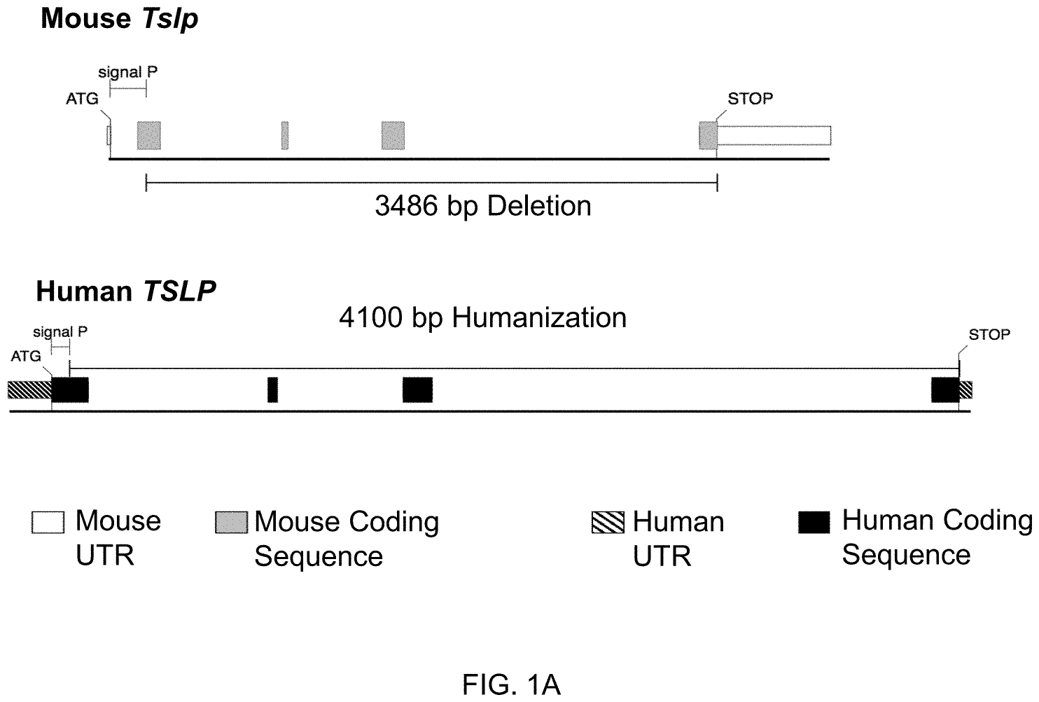

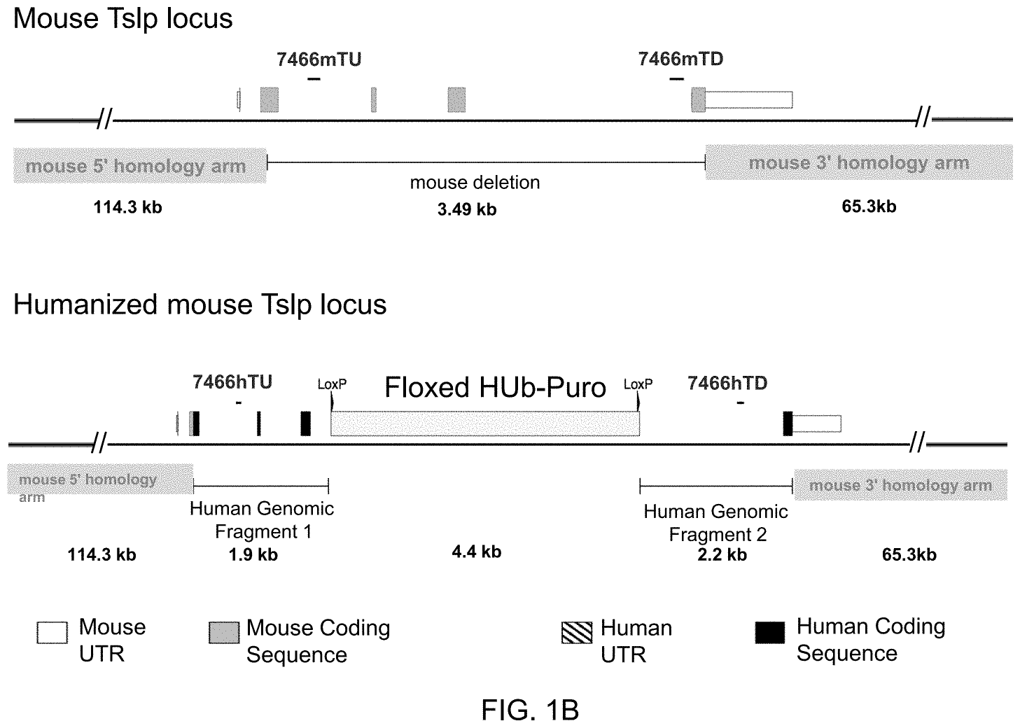

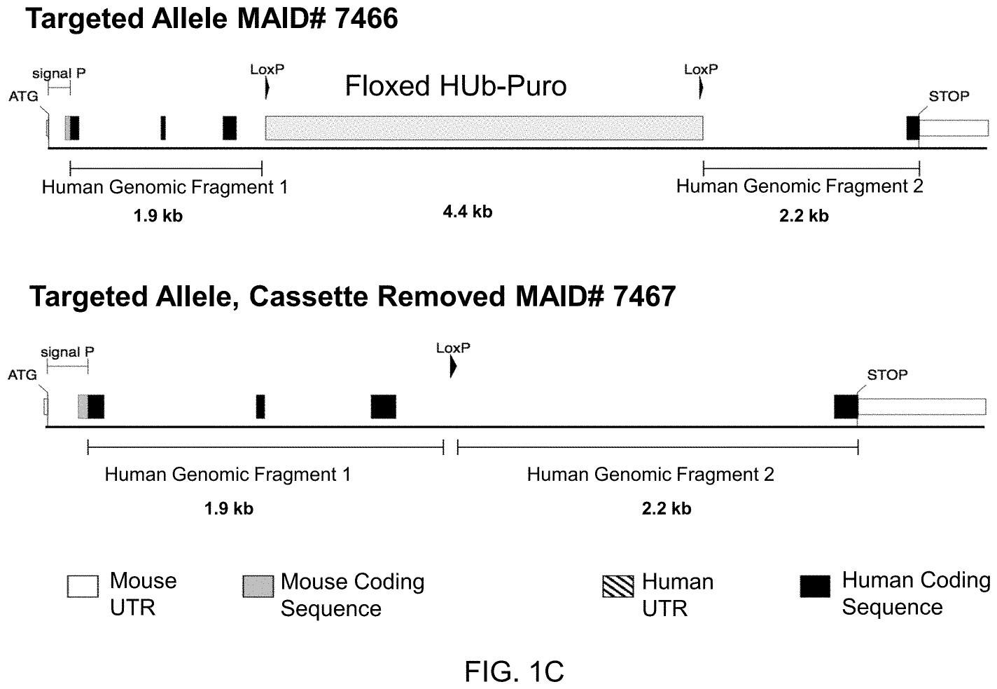

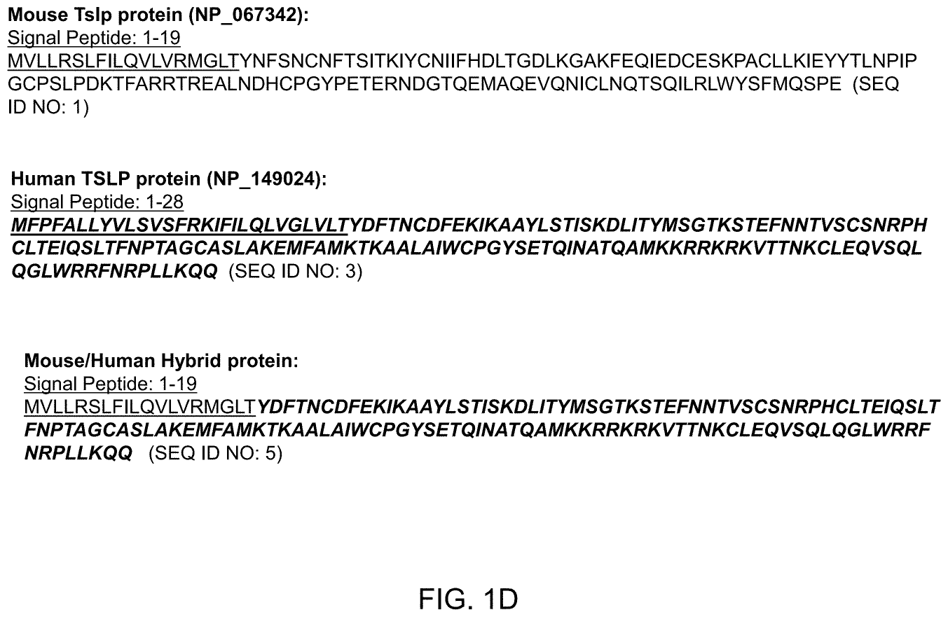

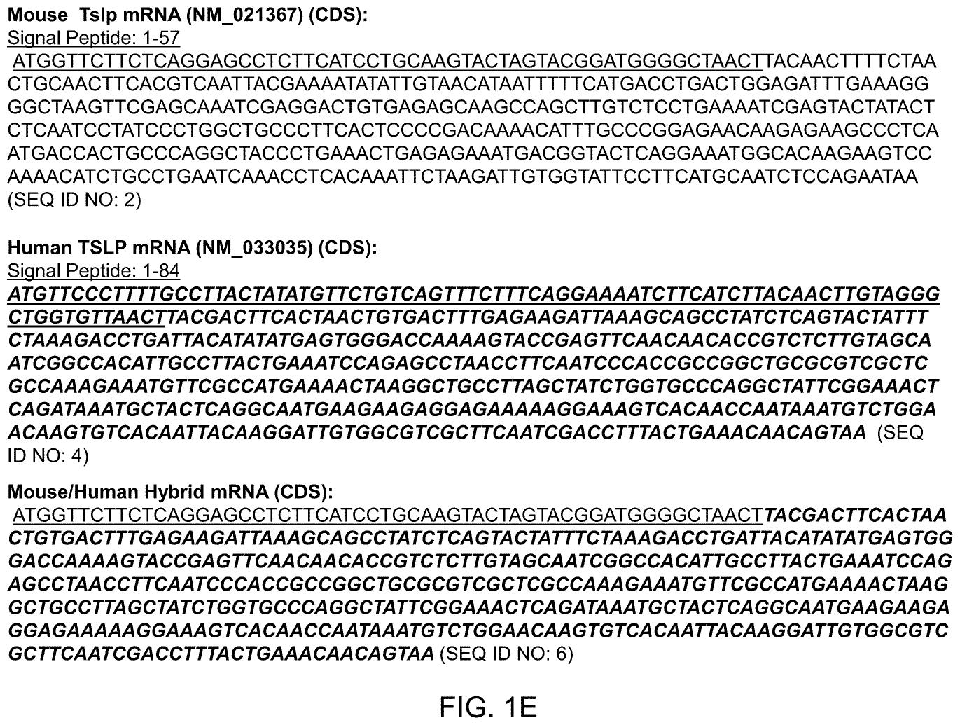

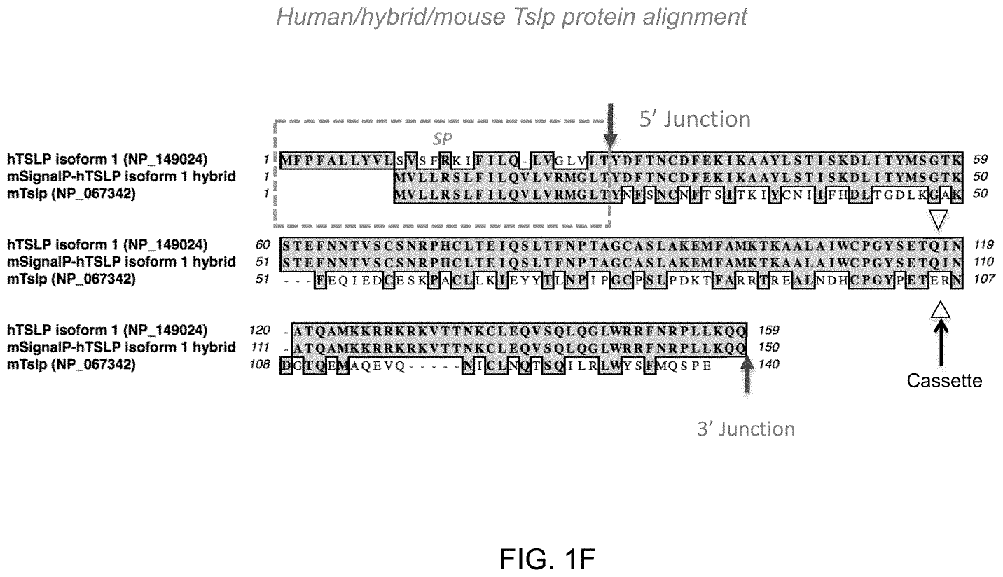

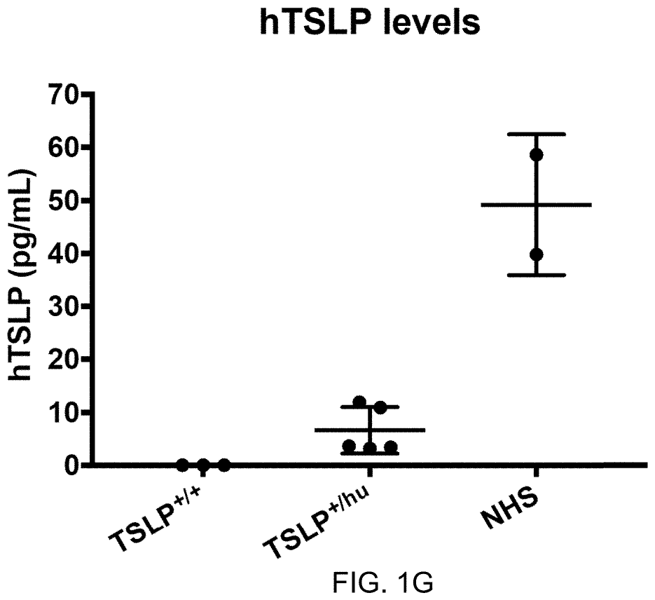

Exemplary sequences, including nucleic acid and protein sequences for human TSLP isoform 1, mouse Tslp, rat Tslp, and humanized Tslp are disclosed in the Sequence Listing and summarized in Table 1. Mouse and rat Tslp genes have a small coding exon 1 and a total of 5 exons, instead of 4 exons as in human TSLP gene. An alignment of human TSLP isoform 1, mouse Tslp, and humanized (hybrid) Tslp protein sequences is provided in FIG. 1F.

TABLE 1

| SEQ | | |
|---|---|---|
| ID NO | Description | Features |
| 1 | Mus musculus Tslp | Length: 140 aa |
| | Protein, NP_067342 | Signal peptide: 1-19 |
| | | Mature protein: 20-140 |
| 2 | Mus musculus Tslp mRNA | Length: 423 bp |
| | (CDS), NM_021367 | |
| 3 | Homo sapiens TSLP protein | Length: 159 aa |
| | isoform 1, NP_149024 | Signal peptide: aa 1-28 |
| | | Mature: aa 29-159 |
| 4 | Homo sapiens TSLP mRNA | Length: 480 bp |
| | (CDS), isoform 1, NM_033035 | |
| 5 | Humanized mouse/human | Length: 150 aa |
| | chimeric Tslp Protein | Signal peptide: 1-19 |
| | | (from mouse) |
| | | Mature protein: 20-150 |
| | | (from human) |
| 6 | Humanized mouse/human | Length: 453 bp |
| | chimeric Tslp CDS | |
| 7 | Rattus norvegicus Tslp | Length: 136 aa |
| | Protein, XP_008770274 | |
| 8 | Rattus norvegicus Tslp mRNA, | Length: 411 bp |
| | XM_008772052 | |

In some embodiments, the rodents disclosed herein comprise a humanized Tslp gene in the germline.

In some embodiments, a rodent disclosed herein comprises a humanized Tslp gene in its genome that includes a nucleotide sequence of a rodent Tslp gene and a nucleotide sequence of a human TSLP gene. As used herein, “a nucleotide sequence of a gene” includes a genomic sequence, an mRNA or cDNA sequence, in full or in part of the gene. For example, a nucleotide sequence of a human TSLP gene can be a genomic sequence, an mRNA sequence, or a cDNA sequence, in full or in part of the human TSLP gene; and a nucleotide sequence of a rodent Tslp gene can be a genomic sequence, an mRNA sequence, or a cDNA sequence, in full or in part of the rodent Tslp gene (e.g., an endogenous rodent Tslp gene). The nucleotide sequence of the rodent Tslp gene and the nucleotide sequence of the human TSLP gene are operably linked to each other such that the humanized Tslp gene in the rodent genome encodes a humanized Tslp protein that performs the functions of a Tslp protein, e.g., binding to a Tslp receptor (Tslpr).

“Human TSLP” gene and protein, as used herein, refers to TSLP gene and protein of the human origin.

In some embodiments, a human TSLP protein comprises the amino acid sequence of SEQ ID NO: 3. In some embodiments, a human TSLP protein comprises an amino acid sequence at least 95% identical to the the amino acid sequence of SEQ ID NO: 3. In some embodiments, a human TSLP protein comprises an amino acid sequence at least 98% identical to the the amino acid sequence of SEQ ID NO: 3. In some embodiments, a human TSLP protein comprises an amino acid sequence at least 99% identical to the the amino acid sequence of SEQ ID NO: 3.

“Rodent Tslp” gene and protein, as used herein, refers to Tslp gene and protein of a rodent (e.g., mouse or rat) origin.

In some embodiments, a mouse Tslp protein comprises the amino acid sequence of SEQ ID NO: 1. In some embodiments, a mouse Tslp protein comprises an amino acid sequence at least 95% identical to the the amino acid sequence of SEQ ID NO: 1. In some embodiments, a mouse Tslp protein comprises an amino acid sequence at least 98% identical to the the amino acid sequence of SEQ ID NO: 1. In some embodiments, a mouse Tslp protein comprises an amino acid sequence at least 99% identical to the the amino acid sequence of SEQ ID NO: 1.

In some embodiments, a rat Tslp protein comprises the amino acid sequence of SEQ ID NO: 7. In some embodiments, a rat Tslp protein comprises an amino acid sequence at least 95% identical to the the amino acid sequence of SEQ ID NO: 7. In some embodiments, a rat Tslp protein comprises an amino acid sequence at least 98% identical to the the amino acid sequence of SEQ ID NO: 7. In some embodiments, a rat Tslp protein comprises an amino acid sequence at least 99% identical to the the amino acid sequence of SEQ ID NO: 7.

In some embodiments, a genetically modified rodent comprises a humanized Tslp gene in its genome, wherein the humanized Tslp gene encodes a humanized Tslp protein that comprises a mature protein sequence that is substantially identical with the mature protein sequence of a human TSLP protein (such as the human TSLP protein as set forth in SEQ ID NO: 3).

A “mature protein” refers to the portion of a protein after an N-terminal signal peptide has been cleaved.

A mature protein sequence that is substantially identical with the mature protein sequence of a human TSLP protein can be (i) a polypeptide sequence that is at least 95% identical with the mature protein of a human TSLP protein, a polypeptide sequence that is at least 98% identical with the mature protein of a human TSLP protein, or a polypeptide sequence that is at least 99% identical with the mature protein of a human TSLP protein. A mature protein sequence that is substantially identical with the mature protein sequence of a human TSLP protein may be a polypeptide sequence identical with the mature protein sequence of a human TSLP protein. A mature protein sequence that is substantially identical with the mature protein sequence of a human TSLP protein may alternatively or additionally be (ii) a polypeptide sequence that differs from the mature protein sequence of a human TSLP protein by not more than 5 amino acids, a polypeptide sequence that differs from the mature protein sequence of a human TSLP protein by not more than 4 amino acids, a polypeptide sequence that differs from the mature protein sequence of a human TSLP protein by not more than 3 amino acids, a polypeptide sequence that differs from the mature protein sequence of a human TSLP protein by not more than 2 amino acids, or a polypeptide sequence that differs from the mature protein sequence of a human TSLP protein by not more than 1 amino acid. A mature protein sequence that is substantially identical with the mature protein sequence of a human TSLP protein may alternatively or additionally be (iii) a polypeptide that differs from the mature protein sequence of a human TSLP protein only at the N- or C-terminal portion of the domain, e.g., by having addition, deletion or substitution of amino acids (not more than 5 amino acids) at the N- or C-terminal portion of the mature protein; by “the N- or C-terminal portion of the mature protein” is meant within 5-10 amino acids from the N- or C-terminus of the mature protein. A mature protein sequence that is substantially identical with the mature protein sequence of a human TSLP protein may alternatively or additionally be (iv) a polypeptide having one or more features described in (i)-(iii) above, e.g., a polypeptide that is at least 95% identical with the mature protein of a human TSLP protein and differs from the mature protein sequence of a human TSLP protein only at the N- or C-terminal portion of the domain by not more than 5 amino acids, or a polypeptide that is at least 98% identical with the mature protein of a human TSLP protein and differs from the mature protein sequence of a human TSLP protein only at the N- or C-terminal portion of the domain by not more than 3 amino acids.

In some embodiments, a human TSLP protein is the human TSLP protein isoform 1 as set forth in SEQ ID NO: 3, with amino acids 29-159 constituting the mature protein sequence. Accordingly, in some embodiments, a genetically modified rodent comprises a humanized Tslp gene in its genome that encodes a humanized Tslp protein that comprises a mature protein sequence that is substantially identical with the amino acid sequence as set forth in amino acids 29-159 of SEQ ID NO: 3. In some embodiments, a humanized Tslp protein comprises a mature protein sequence which comprises amino acids 29-159 of SEQ ID NO: 3. In some embodiments, a humanized Tslp protein comprises a mature protein sequence which comprises amino acids 30-159 of SEQ ID NO: 3. In some embodiments, a humanized Tslp protein comprises a mature protein sequence which comprises amino acids 31-159 of SEQ ID NO: 3. In some embodiments, a humanized Tslp protein comprises a mature protein sequence which comprises amino acids 32-159 of SEQ ID NO: 3. In some embodiments, a humanized Tslp protein comprises a mature protein sequence which comprises amino acids 29-158 of SEQ ID NO: 3. In some embodiments, a humanized Tslp protein comprises a mature protein sequence which comprises amino acids 29-157 of SEQ ID NO: 3. In some embodiments, a humanized Tslp protein comprises a mature protein sequence which comprises amino acids 29-156 of SEQ ID NO: 3. In some embodiments, a humanized Tslp protein comprises a mature protein sequence that is as set forth in amino acids 29-159 of SEQ ID NO: 3.

In some embodiments, the humanized Tslp gene encodes a humanized Tslp protein that comprises a signal peptide that is substantially identical with the signal peptide of a human TSLP protein. In some embodiments, a signal peptide that is substantially identical with the signal peptide of a human TSLP protein is a signal peptide that is at least 95% identical in sequence with the signal peptide of a human TSLP protein. A signal peptide that is substantially identical with the signal peptide of a human TSLP protein may be a signal peptide identical with the signal peptide of a human TSLP protein. Additionally or alternatively, a signal peptide that is substantially identical with the signal peptide of a human TSLP protein may be a signal peptide that differs from the signal peptide of a human TSLP protein by not more than 3 amino acids, by not more than 2 amino acids, or by not more than 1 amino acid. In specific embodiments, the signal peptide of a human TSLP protein comprises the amino acid sequence as set forth in amino acids 1-28 of SEQ ID NO: 3.

In some embodiments, the humanized Tslp gene encodes a humanized Tslp protein that comprises a signal peptide that is substantially identical with the signal peptide of a rodent Tslp protein, such as an endogenous rodent Tslp protein. In some embodiments, a signal peptide that is substantially identical with the signal peptide of a rodent Tslp protein is a signal peptide that is at least 95% identical in sequence with the signal peptide of a rodent Tslp protein; in some embodiments, a signal peptide that is substantially identical with the signal peptide of a rodent Tslp protein is a signal peptide that is identical in sequence with the signal peptide of a rodent Tslp protein. In some embodiments, a signal peptide that is substantially identical with the signal peptide of a rodent Tslp protein is a signal peptide that differs from the signal peptide of a rodent Tslp protein protein by not more than 3 amino acids; in some embodiments, a signal peptide that is substantially identical with the signal peptide of a rodent Tslp protein is a signal peptide that differs from the signal peptide of a rodent Tslp protein protein by not more than 2 amino acids; in some embodiments, a signal peptide that is substantially identical with the signal peptide of a rodent Tslp protein is a signal peptide that differs from the signal peptide of a rodent Tslp protein protein by not more than 1 amino acid. In specific embodiments, a humanized Tslp protein comprises a signal peptide that is substantially identical with the signal peptide of a mouse Tslp protein, e.g., the signal peptide as set forth in amino acids 1-19 of SEQ ID NO: 1. In specific embodiments, a humanized Tslp protein comprises a signal peptide that is substantially identical with the signal peptide of a rat Tslp protein, e.g., the rat Tslp protein as set forth in SEQ ID NO: 7.

As described above, the humanized Tslp gene in the genome of a genetically modified rodent includes a nucleotide sequence of a human TSLP gene (“a human TSLP nucleotide sequence”) and a nucleotide sequence of a rodent Tslp gene (“a rodent Tslp nucleotide sequence”, e.g., an endogenous rodent Tslp nucleotide sequence).

In some embodiments, the human TSLP nucleotide sequence in a humanized Tslp gene encodes at least a substantial portion of the mature protein sequence of a human TSLP protein (e.g., a human TSLP protein isoform 1). A “substantial portion” of a mature Tslp protein sequence refers to a polypeptide that is nearly the full length of the mature protein sequence. In some embodiments, a substantial portion of a mature protein sequence refers to a polypeptide that is at least 95% of the full length mature protein sequence; in some embodiments, a substantial portion of a mature protein sequence refers to a polypeptide that is at least 98% of the full length mature protein sequence; in some embodiments, a substantial portion of a mature protein sequence refers to a polypeptide that is at least 99% of the full length mature protein sequence. In some embodiments, a substantial portion of a mature protein sequence refers to a polypeptide that differs from the mature protein sequence by lacking not more than 5 amino acids at the N- or C-terminus of the mature protein sequence; in some embodiments, a substantial portion of a mature protein sequence refers to a polypeptide that differs from the mature protein sequence by lacking not more than 4 amino acids at the N- or C-terminus of the mature protein sequence; in some embodiments, a substantial portion of a mature protein sequence refers to a polypeptide that differs from the mature protein sequence by lacking not more than 3 amino acids at the N- or C-terminus of the mature protein sequence; a substantial portion of a mature protein sequence refers to a polypeptide that differs from the mature protein sequence by lacking not more than 2 amino acids at the N- or C-terminus of the mature protein sequence; a substantial portion of a mature protein sequence refers to a polypeptide that differs from the mature protein sequence by lacking not more than 1 amino acid at the N- or C-terminus of the mature protein sequence. In some embodiments, the human TSLP nucleotide sequence in a humanized Tslp gene encodes the mature protein sequence of a human TSLP protein (e.g., a human TSLP protein isoform 1, such as the human TSLP protein isoform 1 as set forth in SEQ ID NO: 3). In some embodiments, the human TSLP nucleotide sequence in a humanized Tslp gene encodes amino acids 29-159 of SEQ ID NO: 3. In some embodiments, the human TSLP nucleotide sequence in a humanized Tslp gene encodes amino acids 30-159 of SEQ ID NO: 3. In some embodiments, the human TSLP nucleotide sequence in a humanized Tslp gene encodes amino acids 31-159 of SEQ ID NO: 3. In some embodiments, the human TSLP nucleotide sequence in a humanized Tslp gene encodes amino acids 32-159 of SEQ ID NO: 3. In some embodiments, the human TSLP nucleotide sequence in a humanized Tslp gene encodes amino acids 29-158 of SEQ ID NO: 3. In some embodiments, the human TSLP nucleotide sequence in a humanized Tslp gene encodes amino acids 29-157 of SEQ ID NO: 3. In some embodiments, the human TSLP nucleotide sequence in a humanized Tslp gene encodes amino acids 29-156 of SEQ ID NO: 3.

In some embodiments, the human TSLP nucleotide sequence in a humanized Tslp gene is a cDNA sequence. In some embodiments, the human TSLP nucleotide sequence is a genomic fragment of a human TSLP gene. In some embodiments, the human TSLP nucleotide sequence is a genomic fragment comprising exonic sequences that encode at least a substantial portion of the mature protein sequence of a human TSLP protein. In some embodiments, the human TSLP nucleotide sequence is a genomic fragment comprising exonic sequences that encode the mature protein sequence of a human TSLP protein (e.g., a human TSLP protein isoform 1, such as the human TSLP protein isoform 1 as set forth in SEQ ID NO: 3). In some embodiments, the human TSLP nucleotide sequence is a genomic fragment of a human TSLP gene, which fragment comprises the mature protein amino acids encoding portion of exon 1, exon 2, exon 3, and the coding portion of exon 4 (i.e., through the STOP codon in exon 4).

In some embodiments, the human TSLP nucleotide sequence is a genomic fragment of a human TSLP gene, which fragment also comprises the 3′ UTR of the human TSLP gene (a 3′ portion of human TSLP exon 4).

In some embodiments, the rodent Tslp nucleotide sequence in a humanized Tslp gene encodes a polypeptide substantially identical to the signal peptide of a rodent Tslp protein (e.g., an endogenous rodent Tslp protein). In some embodiments, a polypeptide substantially identical to the signal peptide of a rodent Tslp protein includes a polypeptide that is at least 95% identical in sequence with the signal peptide of a rodent Tslp protein. In some embodiments, a polypeptide substantially identical to the signal peptide of a rodent Tslp protein includes a polypeptide that differs from the signal peptide of a rodent Tslp protein protein by not more than 3 amino acids. In some embodiments, a polypeptide substantially identical to the signal peptide of a rodent Tslp protein includes a polypeptide that differs from the signal peptide of a rodent Tslp protein protein by not more than 2 amino acids. In some embodiments, a polypeptide substantially identical to the signal peptide of a rodent Tslp protein includes a polypeptide that differs from the signal peptide of a rodent Tslp protein protein by not more than 1 amino acid. In some embodiments, the rodent Tslp nucleotide sequence in a humanized Tslp gene encodes the signal peptide of a rodent Tslp protein (e.g., an endogenous rodent Tslp protein, e.g., mouse or rat Tslp protein). In some embodiments, the rodent Tslp nucleotide sequence comprises exonic sequences of a rodent Tslp gene that encode the signal peptide of the rodent Tslp protein. In some embodiments, the rodent Tslp nucleotide sequence is a mouse Tslp nucleotide sequence, and in some such embodiments, the mouse Tslp nucleotide sequence comprises exon 1 and the signal peptide amino acid-coding portion of exon 2 of a mouse Tslp gene (e.g., an endogenous mouse Tslp gene). In some embodiments, the rodent Tslp nucleotide sequence in a humanized Tslp gene comprises the 5′ UTR of a rodent Tslp gene (e.g., a mouse or rat Tslp gene, such as an endogenous mouse or rat Tslp gene).

In some embodiments, the humanized Tslp gene is operably linked to rodent Tslp regulatory sequences, e.g., 5′ transcriptional regulatory sequence(s) such as promoter and/or enhancer of a rodent Tslp gene, such as endogenous rodent 5′ transcriptional regulatory sequence(s) at an endogenous rodent Tslp locus, such that expression of the humanized Tslp gene is under control of the rodent Tslp 5′ regulatory sequence(s).

In some embodiments, the humanized Tslp gene is at an endogenous rodent Tslp locus. In some embodiments, the humanized Tslp gene is at a locus other than an endogenous rodent Tslp locus; e.g., as a result of random integration. In some embodiments where the humanized Tslp gene is at a locus other than an endogenous rodent Tslp locus, the rodents are incapable of expressing a rodent Tslp protein, e.g., as a result of inactivation (e.g., deletion in full or in part) of the endogenous rodent Tslp gene.

In some embodiments where a humanized Tslp gene is at an endogenous rodent Tslp locus, the humanized Tslp gene may result from a replacement of a nucleotide sequence of an endogenous rodent Tslp gene at the endogenous rodent Tslp locus with a nucleotide sequence of a human TSLP gene.

In some embodiments, the nucleotide sequence of a human TSLP gene that replaces a genomic fragment of a rodent Tslp gene at an endogenous rodent Tslp locus is a cDNA sequence. In some embodiments, the human TSLP nucleotide sequence that replaces a genomic fragment of a rodent Tslp gene at an endogenous rodent Tslp locus is a genomic fragment of a human TSLP gene. In some embodiments, the human TSLP nucleotide sequence encode at least a substantial portion of the mature protein sequence of the human TSLP protein. In some embodiments, the human TSLP nucleotide sequence is a genomic fragment of a human TSLP gene that includes includes exons, in full or in part, of a human TSLP gene, that encode at least a substantial portion of the mature protein sequence of the human TSLP protein. In some embodiments, the human genomic fragment comprises the mature protein amino acids encoding portion of exon 1, exon 2, exon 3, and the coding portion of exon 4 of a human TSLP gene. In some embodiments, the human genomic fragment can further comprise the 3′ UTR portion of exon 4 of a human TSLP gene.

In some embodiments, the human nucleotide sequence integrated at an endogenous rodent Tslp locus is operably linked to a rodent Tslp nucleotide sequence that encodes a polypeptide substantially identical with the signal peptide of a rodent Tslp protein. In some embodiments, the human nucleotide sequence integrated at an endogenous rodent Tslp locus is operably linked to an endogenous rodent Tslp genomic sequence that encodes substantially the signal peptide of the endogenous rodent Tslp protein. In some embodiments, the human nucleotide sequence is integrated at an endogenous mouse Tslp locus and is operably linked to a mouse Tslp nucleotide sequence that encodes a polypeptide substantially identical with the signal peptide of a mouse Tslp protein; and in some such embodiments, the mouse Tslp nucleotide sequence comprises exon 1 and the signal peptide amino acid-coding portion of exon 2 of a mouse Tslp gene (e.g., the endogenous mouse Tslp gene).

In some embodiments, a genomic fragment comprising exonic sequences coding for the mature protein sequence of an endogenous rodent Tslp protein at an endogenous rodent Tslp locus (e.g., a mouse genomic fragment that begins from the codon in exon 2 coding for the first amino acid of the mature mouse Tslp protein through the STOP codon) has been replaced with a genomic fragment of a human TSLP gene comprising exonic sequences coding for the mature protein sequence of the human TSLP protein (e.g., a genomic fragment that begins from the codon in exon 1 coding for the first amino acid of the mature human TSLP protein through the STOP codon in exon 4). As a result, a humanized Tslp gene is formed at the endogenous rodent Tslp locus. In some embodiments, a humanized Tslp gene is formed at an endogenous mouse Tslp locus and comprises mouse Tslp exon 1, the signal peptide amino acid coding portion of mouse Tslp exon 2, the codon in human TSLP exon 1 coding for the first amino acid of the mature human TSLP protein through the STOP codon in human TSLP exon 4, and the mouse 3′ UTR in mouse Tslp exon 5. Such humanized Tslp gene encodes a humanized Tslp protein that comprises a mouse Tslp signal peptide and a mature human TSLP polypeptide.

In some embodiments, a rodent provided herein is heterozygous for a humanized Tslp gene in its genome. In some embodiments, a rodent provided herein is homozygous for a humanized Tslp gene in its genome.

In some embodiments, a humanized Tslp gene results in an expression of the encoded humanized Tslp protein in a rodent, e.g., in the serum of the rodent. In some embodiments, a humanized Tslp protein is expressed in cells and tissues in which a counterpart rodent Tslp protein is expressed in a control rodent (e.g., a rodent without the humanized Tslp gene); e.g., epithelial cells and keratinocytes in the skin, gut, lungs and ocular tissue, as well as dendritic cells, mast cells and basophils. See, e.g., Tsilingiri et al. (2017), supra.

In some embodiments, rodents disclosed herein are incapable of expressing a rodent Tslp protein, e.g., as a result of inactivation (e.g., deletion in full or in part) or replacement (in full or in part) of the endogenous rodent Tslp gene.

### TSLPR Humanization

TSLP acts through a heterodimer composed of a chain specific for TSLP (referred to as “TSLPR” or “Tslpr”) and the IL7 receptor a chain. TSLPR contains a signal peptide, an extracellular domain (“ECD” or “ectodomain”), a transmembrane domain and an intracellular (cytoplasmic) domain.

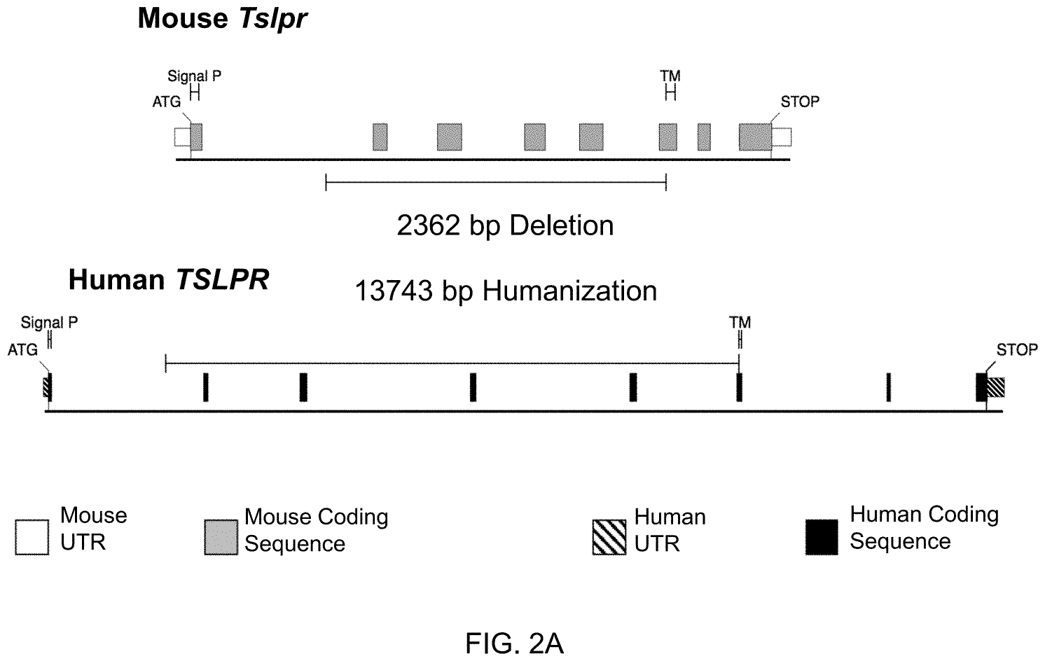

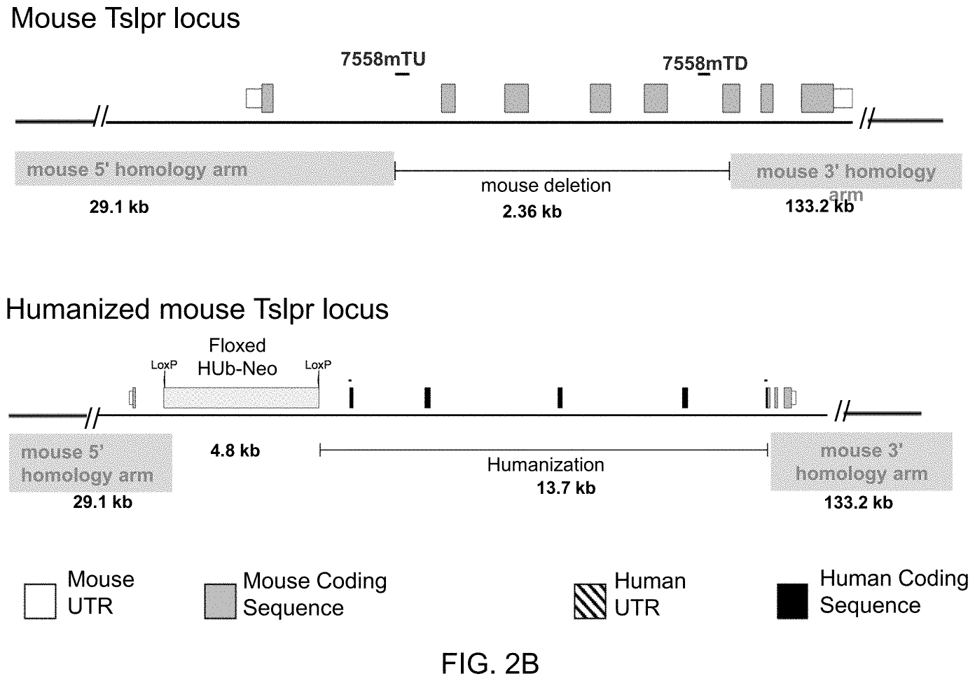

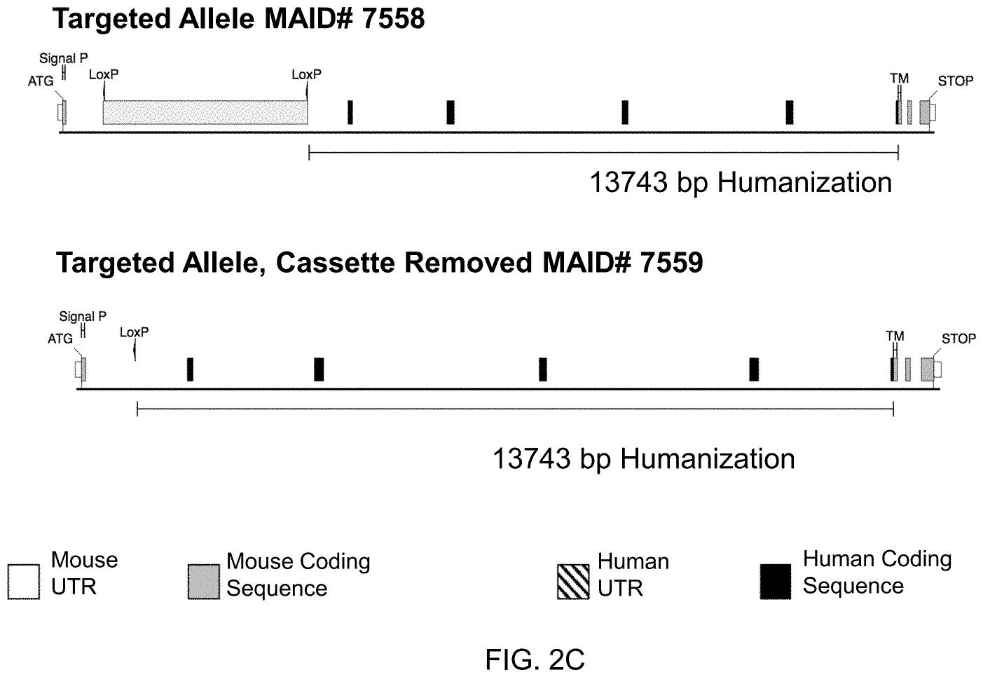

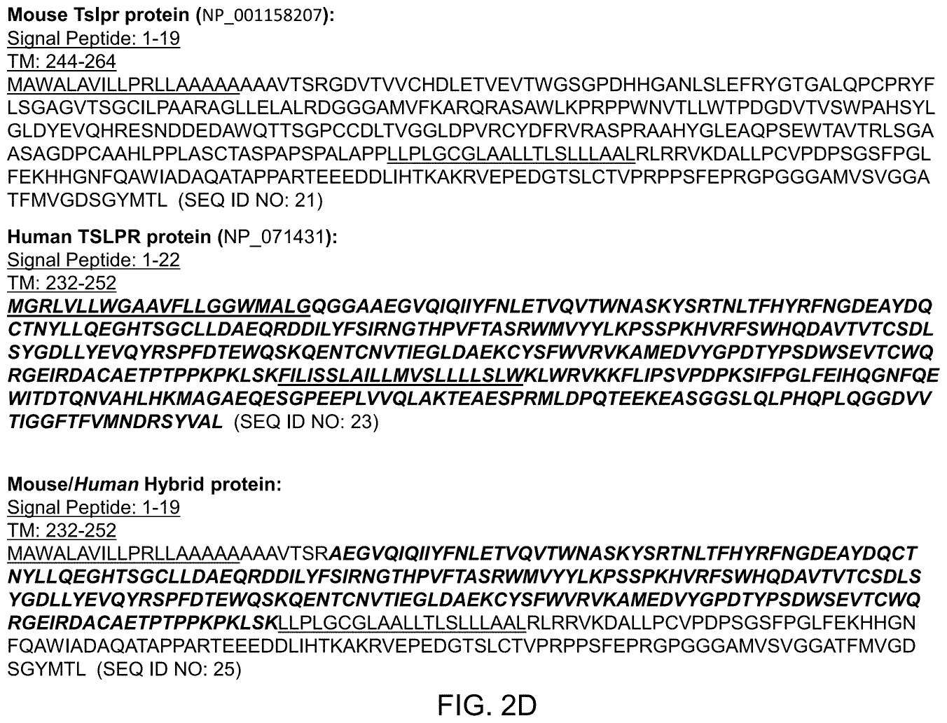

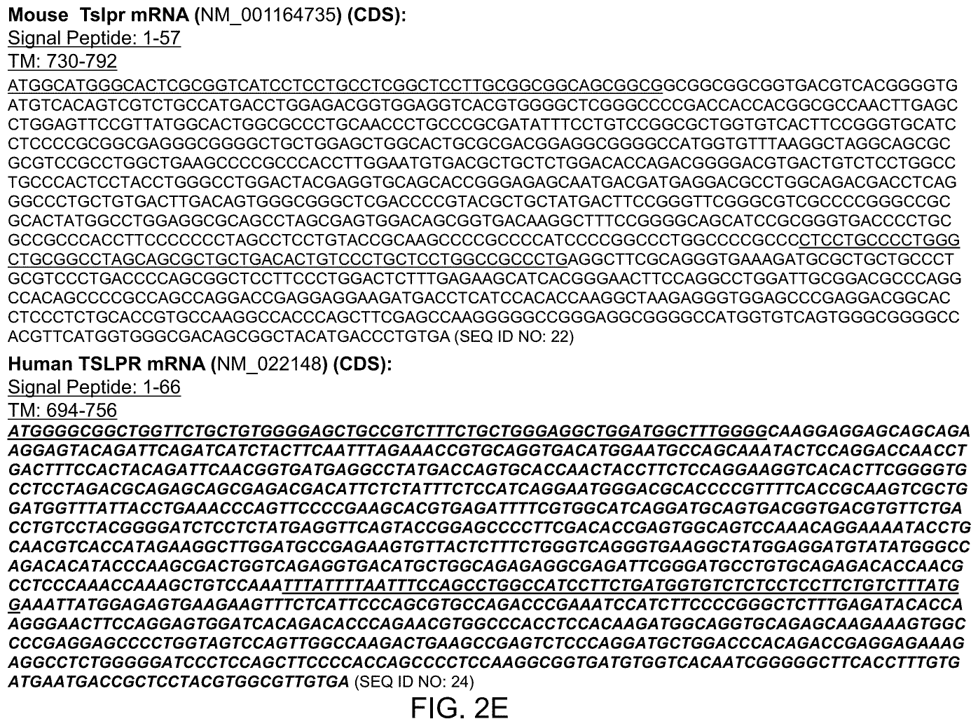

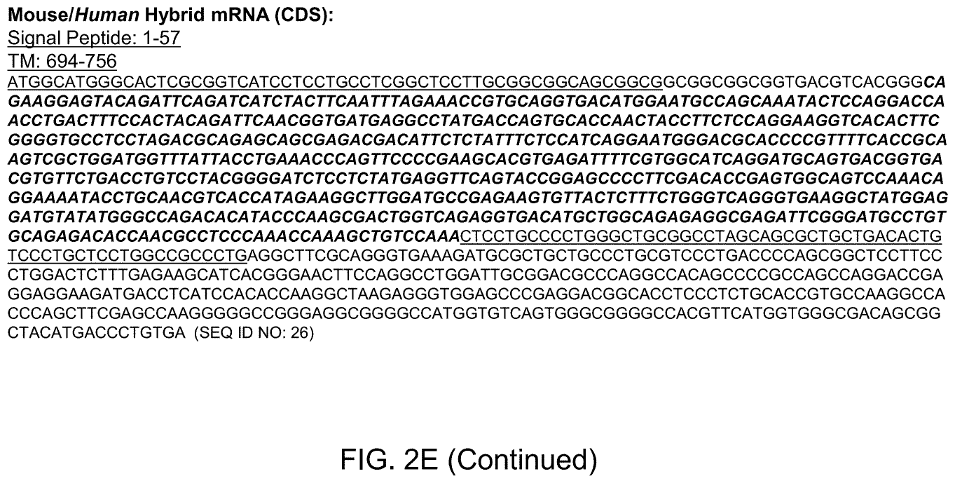

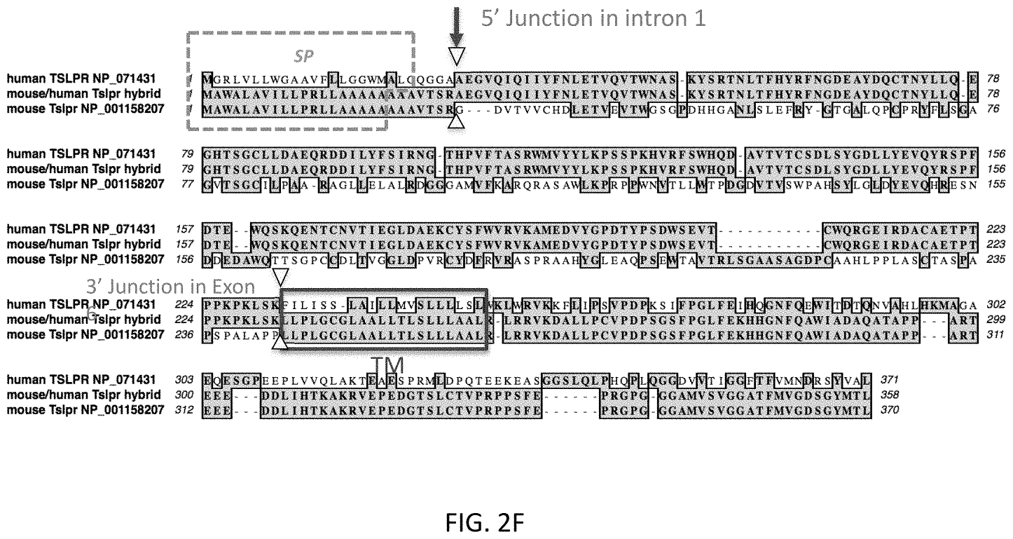

Exemplary sequences, including nucleic acid and protein sequences for human TSLPR isoform 1, mouse Tslpr isoform 1, rat Tslpr isoform 1, and humanized (mouse-human hybrid) Tslpr, are disclosed in the Sequence Listing and summarized in Table 2. An alignment of human TSLPR isoform 1, mouse Tslpr isoform 1, and humanized (mouse-human hybrid) Tslpr protein sequences is provided in FIG. 2F. Unless specifically indicated, the exon numberings of human and mouse genes are based on exons encoding human and mouse protein isoform 1.

TABLE 2

| SEQ | | |
|---|---|---|
| ID NO | Description | Features |
| 21 | Mus musculus Tslpr isoform 1 | Length: 370 aa |
| | Protein, NP_001158207 | Signal peptide: aa 1-19 |
| | | Ectodomain: aa 20-243 |
| | | Transmembrane: aa 244-264 |
| | | Intracellular: aa 265-370 |
| 22 | Mus musculus Tslpr mRNA | Length: 1113 bp |
| | (CDS), isoform 1, | |
| | NM_001164735 | |
| 23 | Homo sapiens TSLPR protein | Length: 371 aa |
| | isoform 1, NP_071431 | Signal peptide: aa 1-22 |
| | | Ectodomain: aa 23-231 |
| | | Transmembrane: aa 232-252 |
| | | Intracellular: aa 253-371 |
| 24 | Homo sapiens TSLPR mRNA | Length: 1116 bp |
| | (CDS), isoform 1, NM_022148 | |
| 25 | Humanized mouse/human | Length: 358 aa |
| | chimeric Tslpr Protein | Signal peptide: 1-19 |
| | | (from mouse) |
| | | Ectodomain: aa 20-231 |
| | | (aa 20-26 from mouse and |
| | | aa 27-231 from human) |
| | | Transmembrane: aa 232-252 |
| | | (from mouse) |
| | | Intracellular: aa 253-358 |
| | | (from mouse) |
| 26 | Humanized mouse/human | Length: 1077 bp |
| | chimeric Tslpr mRNA (CDS) | |
| 27 | Rattus norvegicus Tslpr | Length: 360 aa |
| | Protein, AAL90454.1. | |
| 28 | Rattus norvegicus Tslpr mRNA, | Length: 1209 bp |
| | AF404510.1 | |

In some embodiments, the rodents disclosed herein comprise a humanized Tslpr gene in the germline.

In some embodiments, a rodent disclosed herein comprises a humanized Tslpr gene in its genome that includes a nucleotide sequence of a rodent Tslpr gene (e.g., an endogenous rodent Tslpr gene) and a nucleotide sequence of a human TSLPR gene. As used herein, “a nucleotide sequence of a gene” includes a genomic sequence, an mRNA or cDNA sequence, in full or in part of the gene. As a non-limiting example, a nucleotide sequence of a human TSLPR gene includes a genomic sequence, an mRNA or cDNA sequence, in full or in part of the human TSLPR gene. The nucleotide sequence of the rodent Tslpr gene and the nucleotide sequence of the human TSLPR gene are operably linked to each other such that the humanized Tslpr gene in the rodent genome encodes a humanized Tslpr protein that has a Tslpr protein structure (comprising an ectodomain, a transmembrane domain and a cytoplasmic domain) and performs Tslpr functions (e.g., binds a Tslp protein and forms a heterodimer with IL-7 receptor).

“Human TSLPR” gene and protein, as used herein, refers to TSLPR gene and protein of the human origin. In some embodiments, a human TSLPR protein comprises the the amino acid sequence of SEQ ID NO: 23. In some embodiments, a human TSLPR protein comprises an amino acid sequence at least 95% identical to the the amino acid sequence of SEQ ID NO: 23. In some embodiments, a human TSLPR protein comprises an amino acid sequence at least 98% identical to the the amino acid sequence of SEQ ID NO: 23. In some embodiments, a human TSLPR protein comprises an amino acid sequence at least 99% identical to the the amino acid sequence of SEQ ID NO: 23.

“Rodent Tslpr” gene and protein, as used herein, refers to Tslpr gene and protein of a rodent (e.g., mouse or rat) origin. In some embodiments, a mouse Tslpr protein comprises the the amino acid sequence of SEQ ID NO: 21. In some embodiments, a mouse Tslpr protein comprises an amino acid sequence at least 95% identical to the the amino acid sequence of SEQ ID NO: 21. In some embodiments, a mouse Tslpr protein comprises an amino acid sequence at least 98% identical to the the amino acid sequence of SEQ ID NO: 21. In some embodiments, a mouse Tslpr protein comprises an amino acid sequence at least 99% identical to the the amino acid sequence of SEQ ID NO: 21. In some embodiments, a rat Tslpr protein comprises the the amino acid sequence of SEQ ID NO: 27. In some embodiments, a rat Tslpr protein comprises an amino acid sequence at least 95% identical to the the amino acid sequence of SEQ ID NO: 27. In some embodiments, a rat Tslpr protein comprises an amino acid sequence at least 98% identical to the the amino acid sequence of SEQ ID NO: 27. In some embodiments, a rat Tslpr protein comprises an amino acid sequence at least 99% identical to the the amino acid sequence of SEQ ID NO: 27.

In some embodiments, a genetically modified rodent contains a humanized Tslpr gene in its genome, wherein the humanized Tslpr gene encodes a humanized Tslpr protein that contains an ectodomain that is substantially identical with the ectodomain of a human TSLPR protein. In some embodiments, an ectodomain that is substantially identical with the ectodomain of a human TSLPR protein exhibits the same functionality (e.g., ligand binding properties) as the ectodomain of a human TSLPR protein. An ectodomain or polypeptide that is “substantially identical with the ectodomain of a human TSLPR protein” can be (i) a polypeptide that is at least 95% identical in sequence with the ectodomain of a human TSLPR protein, a polypeptide that is at least 98% identical in sequence with the ectodomain of a human TSLPR protein, or a polypeptide that is at least 99%, identical in sequence with the ectodomain of a human TSLPR protein. An ectodomain or polypeptide that is “substantially identical with the ectodomain of a human TSLPR protein” can be a polypeptide that is 100% identical in sequence with the ectodomain of a human TSLPR protein. Alternatively or additionally, an ectodomain or polypeptide that is “substantially identical with the ectodomain of a human TSLPR protein” can be (ii) a polypeptide that differs from the ectodomain of a human TSLPR protein by not more than 10 amino acids, a polypeptide that differs from the ectodomain of a human TSLPR protein by not more than 7 amino acids, a polypeptide that differs from the ectodomain of a human TSLPR protein by not more than 5 amino acids, a polypeptide that differs from the ectodomain of a human TSLPR protein by not more than 4 amino acids, a polypeptide that differs from the ectodomain of a human TSLPR protein by not more than 3 amino acids, a polypeptide that differs from the ectodomain of a human TSLPR protein by not more than 2 amino acids, a polypeptide that differs from the ectodomain of a human TSLPR protein by not more than 1 amino acid. Alternatively or additionally, an ectodomain or polypeptide that is “substantially identical with the ectodomain of a human TSLPR protein” can be (iii) a polypeptide that differs from the ectodomain of a human TSLPR protein only at the N- or C-terminal portion of the ectodomain, e.g., by having addition, deletion and/or substitution of amino acids at the N- and/or C-terminal portion of the ectodomain (i.e., within 5-10 amino acids from the N or C terminus of the ectodomain). Alternatively or additionally, an ectodomain or polypeptide that is “substantially identical with the ectodomain of a human TSLPR protein” can be (iv) a polypeptide that has one or more of the features delineated in (i)-(iii) above, e.g., a polypeptide that is at least 95% identical in sequence with the ectodomain of a human TSLPR protein and differs from the ectodomain of the human TSLPR protein only at the N- or C-terminal portion of the ectodomain by not more than 10 amino acids, a polypeptide that is at least 95% identical in sequence with the ectodomain of a human TSLPR protein and differs from the ectodomain of the human TSLPR protein only at the N- or C-terminal portion of the ectodomain by not more than 5 amino acids, or a polypeptide that is at least 98% identical in sequence with the ectodomain of a human TSLPR protein and differs from the ectodomain of the human TSLPR protein only at the N- or C-terminal portion of the ectodomain by not more than 3 amino acids. In some embodiments, a human TSLPR protein comprises the amino acid sequence as set forth in SEQ ID NO: 23, and its ectodomain is composed of amino acids 23-231 of SEQ ID NO: 23. In some embodiments, the humanized Tslpr gene encodes a humanized Tslpr protein whose ectodomain is substantially identical with the ectodomain of the human TSLPR protein as set forth in SEQ ID NO: 23, i.e., substantially identical with amino acids 23-231 of SEQ ID NO: 23. For example, the humanized Tslpr gene encodes a humanized Tslpr protein having an ectodomain that comprises amino acids 23-231, 24-231, 25-231, 26-231, 27-231, 28-231, 23-230, 23-229, 23-228, 23-227 or 23-226 of SEQ ID NO: 23. In some embodiments, the humanized Tslpr gene encodes a humanized Tslpr protein having an ectodomain that comprises amino acids 23-231 of SEQ ID NO: 23. In some embodiments, the humanized Tslpr gene encodes a humanized Tslpr protein having an ectodomain that comprises amino acids 25-231 of SEQ ID NO: 23. In some embodiments, the humanized Tslpr gene encodes a humanized Tslpr protein having an ectodomain that comprises amino acids 27-231 of SEQ ID NO: 23. In some embodiments, the humanized Tslpr gene encodes a humanized Tslpr protein having an ectodomain that comprises amino acids 23-228 of SEQ ID NO: 23. In some embodiments, the humanized Tslpr gene encodes a humanized Tslpr protein having an ectodomain that comprises amino acids 23-226 of SEQ ID NO: 23.

In some embodiments, the humanized Tslpr gene encodes a humanized Tslpr protein having an ectodomain that comprises amino acids from the N-terminus of the ectodomain of a rodent Tslpr (e.g., an endogenous rodent Tslpr), followed by the ectodomain of a human TSLPR or a substantial portion thereof. A “substantial portion of the ectodomain” of a human TSLPR (or a rodent Tslpr) protein refers to a polypeptide that is nearly the full ectodomain of the protein. In some embodiments, a substantial portion of the ectodomain of a human TSLPR protein refers to a polypeptide that is at least 95% of the full length ectodomain of the human TSLPR protein. In some embodiments, a substantial portion of the ectodomain of a human TSLPR protein refers to a polypeptide that is at least 98% of the full length ectodomain of the human TSLPR protein. Sequence. In some embodiments, a substantial portion of the ectodomain of a human TSLPR protein refers to a polypeptide that differs from the full length ectodomain by lacking not more than 10 amino acids at the N- or C-terminus of the ectodomain. In some embodiments, a substantial portion of the ectodomain of a human TSLPR protein refers to a polypeptide that differs from the full length ectodomain by lacking not more than 5 amino acids at the N- or C-terminus of the ectodomain. In some embodiments, a substantial portion of the ectodomain of a human TSLPR protein refers to a polypeptide that differs from the full length ectodomain by lacking not more than 4 amino acids at the N- or C-terminus of the ectodomain. For example, the ectodomain of the human TSLPR protein as set forth in SEQ ID NO: 23 is defined by amino acids 23-231, and examples of a substantial portion of the ectodomain can include amino acids 25-231, 26-231, 27-231, 23-228, 23-227, or 23-226 of SEQ ID NO: 23. In some embodiments, a substantial portion of the ectodomain of a human TSLPR protein comprises amino acids 25-231 of SEQ ID NO: 23. In some embodiments, a substantial portion of the ectodomain of a human TSLPR protein comprises amino acids 27-231 of SEQ ID NO: 23. In some embodiments, a substantial portion of the ectodomain of a human TSLPR protein comprises amino acids 23-228 of SEQ ID NO: 23. In some embodiments, a substantial portion of the ectodomain of a human TSLPR protein comprises amino acids 23-226 of SEQ ID NO: 23. In some embodiments, the ectodomain of a humanized Tslpr protein comprises 6-8 amino acids from the N-terminus of the ectodomain of a rodent Tslpr (e.g., an endogenous rodent Tslpr), followed by a substantial portion of the ectodomain of a human TSLPR protein. In some embodiments, the ectodomain of a humanized Tslpr protein comprises 7 amino acids from the N-terminus of the ectodomain of a rodent Tslpr (e.g., an endogenous rodent Tslpr), followed by amino acids 27-231 of SEQ ID NO: 23. In some embodiments, the humanized Tslpr gene encodes a humanized Tslpr protein that contains an ectodomain as set forth in amino acids 20-231 of SEQ ID NO: 25—this ectodomain comprises 7 amino acids from the N-terminus of a mouse Tslpr ectodomain, followed by amino acids 27-231 of SEQ ID NO: 23 (human Tslpr); and differs from the ectodomain of the human TSLPR protein of SEQ ID NO: 23 in the N-terminus of the ectodomain (“AAAVTSR” (SEQ ID NO: 67) in humanized TSLPR of SEQ ID NO: 25, as opposed to “QGGA” (SEQ ID NO: 68) in human TSLPR of SEQ ID NO: 23) but is identical to the human TSLP ectodomain in the remaining ame 205 amino acids.

In some embodiments, the humanized Tslpr gene encodes a humanized Tslpr protein that contains a transmembrane-cytoplasmic sequence (i.e., a sequence that includes both the transmembrane domain and the cytoplasmic domain) that is substantially identical with the transmembrane-cytoplasmic sequence of a rodent Tslpr protein, e.g., an endogenous rodent Tslpr protein. In some embodiments, a transmembrane-cytoplasmic sequence that is substantially identical with the transmembrane-cytoplasmic sequence of an endogenous rodent Tslpr protein exhibits the same functionality (e.g., signal transduction and/or interaction with intracellular molecules) as the transmembrane-cytoplasmic sequence of a rodent Tslpr protein such as an endogenous rodent Tslpr protein. A transmembrane-cytoplasmic sequence or polypeptide that is “substantially identical with the transmembrane-cytoplasmic sequence of a rodent Tslpr protein” can be (i) a polypeptide that is at least 95% identical in sequence with the transmembrane-cytoplasmic sequence of a rodent Tslpr protein, or a polypeptide that is at least 98% identical in sequence with the transmembrane-cytoplasmic sequence of a rodent Tslpr protein. A transmembrane-cytoplasmic sequence or polypeptide that is “substantially identical with the transmembrane-cytoplasmic sequence of a rodent Tslpr protein” can be a polypeptide that is identical with the transmembrane-cytoplasmic sequence of a rodent Tslpr protein. Alternatively or additionally, a transmembrane-cytoplasmic sequence or polypeptide that is “substantially identical with the transmembrane-cytoplasmic sequence of a rodent Tslpr protein” can be (ii) a polypeptide that differs from the transmembrane-cytoplasmic sequence of a rodent Tslpr protein by not more than 5 amino acids, a polypeptide that differs from the transmembrane-cytoplasmic sequence of a rodent Tslpr protein by not more than 4 amino acids, a polypeptide that differs from the transmembrane-cytoplasmic sequence of a rodent Tslpr protein by not more than 3 amino acids, a polypeptide that differs from the transmembrane-cytoplasmic sequence of a rodent Tslpr protein by not more than 2 amino acids, or a polypeptide that differs from the transmembrane-cytoplasmic sequence of a rodent Tslpr protein by not more than 1 amino acid. Alternatively or additionally, a transmembrane-cytoplasmic sequence or polypeptide that is “substantially identical with the transmembrane-cytoplasmic sequence of a rodent Tslpr protein” can be (iii) in some embodiments, a polypeptide that differs from the transmembrane-cytoplasmic sequence of a rodent Tslpr protein only at the N- or C-terminus, e.g., by having addition, deletion or substitution of amino acids at the N- or C-terminal portion of the transmembrane-cytoplasmic sequence. Alternatively or additionally, a transmembrane-cytoplasmic sequence or polypeptide that is “substantially identical with the transmembrane-cytoplasmic sequence of a rodent Tslpr protein” can be; (iv) a polypeptide having one or more features delienated in (i)-(iii) above, e.g., a polypeptide that is at least 95% identical in sequence with the transmembrane-cytoplasmic sequence of a rodent Tslpr protein, and differs from the transmembrane-cytoplasmic sequence of a rodent Tslpr protein only at the N- or C-terminus by not more than 5 amino acids; or a polypeptide that is at least 95% identical in sequence with the transmembrane-cytoplasmic sequence of a rodent Tslpr protein, and differs from the transmembrane-cytoplasmic sequence of a rodent Tslpr protein only at the N- or C-terminus by not more than 3 amino acids. By “the N- or C-terminal portion of the transmembrane-cytoplasmic sequence” is meant within 5-10 amino acids from the N-terminus of the transmembrane domain or within 5-10 amino acids from the C-terminus of the cytoplasmic domain. In some embodiments, a humanized Tslpr protein contains a transmembrane-cytoplasmic sequence that is substantially identical with the transmembrane-cytoplasmic sequence of a mouse Tslpr protein (such as an endogenous mouse Tslpr protein). In some embodiments, a humanized Tslpr protein contains a transmembrane-cytoplasmic sequence that is substantially identical with the transmembrane-cytoplasmic sequence of a rat Tslpr protein (such as an endogenous rat Tslpr protein).

In some embodiments, the humanized Tslpr gene encodes a humanized Tslpr protein that contains a signal peptide that is substantially identical with the signal peptide of a rodent Tslpr protein (e.g., an endogenous rodent Tslpr protein). A signal peptide that is “substantially identical with the signal peptide of a rodent Tslpr protein”, can be (i) a polypeptide that is at least 95% identical in sequence with the signal peptide of a rodent Tslpr protein, or a polypeptide that is identical in sequence with the signal peptide of a rodent Tslpr protein. Alternatively or additionally, a signal peptide that is “substantially identical with the signal peptide of a rodent Tslpr protein” can be (ii) a polypeptide that differs from the signal peptide of an endogenous rodent Tslpr protein by not more than 3 amino acids, a polypeptide that differs from the signal peptide of an endogenous rodent Tslpr protein by not more than 2 amino acids, or a polypeptide that differs from the signal peptide of an endogenous rodent Tslpr protein by not more than 1 amino acids. Alternatively or additionally, a signal peptide that is “substantially identical with the signal peptide of a rodent Tslpr protein” can be a polypeptide that differs from the signal peptide of an endogenous rodent Tslpr protein only at the N- or C-terminus, e.g., by having addition, deletion or substitution of amino acids at the N- or C-terminal portion of the signal peptide. Alternatively or additionally, a signal peptide that is “substantially identical with the signal peptide of a rodent Tslpr protein” can be (iv) a polypeptide having one or more features delineated in (i)-(iii) above, e.g., a polypeptide at least 95% identical with the signal peptide of a rodent Tslpr protein and only differing from the signal peptide of a rodent Tslpr protein at the N- or C-terminus by not more than 3 amino acids. By “the N- or C-terminal portion of the signal peptide” is meant within 5 amino acids from the N- or C-terminus of the signal peptide. In some embodiments, a humanized Tslpr protein includes a signal peptide substantially identical with the signal peptide of a mouse Tslpr protein (such as an endogenous mouse Tslpr protein). In some embodiments, a humanized Tslpr protein includes a signal peptide substantially identical with the signal peptide of a rat Tslpr protein (such as an endogenous rat Tslpr protein).

In some embodiments, the humanized Tslpr gene in the genome of a genetically modified rodent includes a nucleotide sequence of a human TSLPR gene (“a human TSLPR nucleotide sequence”) and a nucleotide sequence of a rodent Tslpr gene (“a rodent Tslpr nucleotide sequence”, such as an endogenous roden Tslpr nucleotide sequence), wherein the human TSLPR nucleotide sequence encodes at least a substantial portion of the ectodomain of a human TSLPR protein. As described above, examples of a substantial portion of the ectodomain of a human TSLPR can include amino acids 25-231, 26-231, 27-231, 23-228, 23-227, or 23-226 of SEQ ID NO: 23. In some embodiments, a substantial portion of the ectodomain comprises amino acids 27-231 of SEQ ID NO: 23. In some embodiments, a substantial portion of the ectodomain comprises amino acids 25-231 of SEQ ID NO: 23. In some embodiments, a substantial portion of the ectodomain comprises amino acids 23-228 of SEQ ID NO: 23. In some embodiments, a substantial portion of the ectodomain comprises amino acids 23-226 of SEQ ID NO: 23. In some embodiments, the human TSLPR nucleotide sequence is a cDNA sequence. In some embodiments, the human TSLPR nucleotide sequence in a humanized Tslpr gene encodes the ectodomain of a human TSLPR protein (e.g., a human TSLPR protein isoform 1 as defined in SEQ ID NO: 23). In some embodiments, the human TSLPR nucleotide sequence is a genomic fragment of a human TSLPR gene. In some embodiments, the human TSLPR nucleotide sequence is a genomic fragment of a human TSLPR gene comprising exon 2 through the codon in exon 6 coding for the last amino acid of the ectodomain of the human TSLPR protein. In some embodiments, the human TSLPR genomic fragment encodes amino acids 27-231 of a human TSLPR isoform 1, absent the 4 amino acids at the N-terminus of the ectodomain of the human TSLPR isoform 1. In some embodiments, the human TSLPR genomic fragment further comprises a 3′ portion of intron 1, operably linked to exon 2 through the codon in exon 6 coding for the last amino acid of the ectodomain of the human TSLPR protein.

In some embodiments, the humanized Tslpr gene in the genome of a genetically modified rodent includes a rodent Tslpr nucleotide sequence and a human TSLPR nucleotide sequence, wherein the rodent Tslpr nucleotide sequence encodes a polypeptide substantially identical to the transmembrane-cytoplasmic sequence of a rodent Tslpr protein (e.g., an endogenous rodent Tslpr protein). In some embodiments, the rodent Tslpr nucleotide sequence present in a humanized Tslpr gene encodes the transmembrane-cytoplasmic sequence of an endogenous rodent Tslpr protein. In some embodiments, the rodent Tslpr nucleotide sequence present in a humanized Tslpr gene is a mouse Tslpr nucleotide sequence; and in some such embodiments, the mouse Tslpr nucleotide sequence comprises a portion of exon 6 (beginning from the codon coding for the first amino acid of the mouse Tslpr transmembrane domain) through exon 8 of a mouse Tslpr gene (e.g., an endogenous mouse Tslpr gene).

In some embodiments, the humanized Tslpr gene in the genome of a genetically modified rodent includes a rodent Tslpr nucleotide sequence upstream (5′) of a human TSLPR nucleotide sequence, wherein the rodent Tslpr nucleotide sequence encodes a polypeptide substantially identical to the signal peptide of a rodent Tslpr protein (e.g., an endogenous rodent Tslpr protein). In some embodiments, the rodent Tslpr nucleotide sequence encoding a polypeptide substantially identical to the signal peptide of a rodent Tslpr protein is a mouse Tslpr nucleotide sequence (e.g., an endogenous mouse Tslpr nucleotide sequence), or a rat Tslpr nucleotide sequence (e.g., an endogenous rat Tslpr nucleotide sequence). In some embodiments, the rodent Tslpr nucleotide sequence encoding a polypeptide that comprises the signal peptide sequence and amino acids (e.g., 6-8 amino acids) from the N-terminus of the ectodomain of a rodent Tslpr protein. In some embodiments, the rodent Tslpr nucleotide sequence encoding a polypeptide that comprises the signal peptide sequence and 7 amino acids from the N-terminus of the ectodomain of a rodent Tslpr protein. In some embodiments, the rodent Tslpr nucleotide sequence is a mouse Tslpr nucleotide sequence which comprises exon 1 of a mouse Tslpr gene (e.g., an endogenous mouse Tslpr gene); and in some such embodiments, the mouse Tslpr nucleotide sequence further comprises a 5′ portion of intron 1 of a mouse Tslpr gene.

In some embodiments, the humanized Tslpr gene is operably linked to rodent Tslpr 5′ regulatory sequences such as endogenous rodent Tslpr regulatory sequences, e.g., a 5′ transcriptional regulatory sequence(s) such as promoter and/or enhancers, such that expression of the humanized Tslpr gene is under control of the rodent Tslpr 5′ regulatory sequence(s).

In some embodiments, the humanized Tslpr gene is at an endogenous rodent Tslpr locus. In some embodiments, the humanized Tslpr gene is at a locus other than an endogenous rodent Tslpr locus; e.g., as a result of random integration. In some embodiments where the humanized Tslpr gene is at a locus other than an endogenous rodent Tslpr locus, the rodents are incapable of expressing a rodent Tslpr protein, e.g., as a result of inactivation (e.g., deletion in full or in part) of the endogenous rodent Tslpr gene.

In some embodiments where a humanized Tslpr gene is at an endogenous rodent Tslpr locus, the humanized Tslpr gene results from a replacement of a nucleotide sequence of an endogenous rodent Tslpr gene at the endogenous rodent Tslpr locus with a nucleotide sequence of a human TSLPR gene.

In some embodiments, the nucleotide sequence of an endogenous rodent Tslpr gene at an endogenous rodent Tslpr locus that is being replaced is a genomic fragment of an endogenous rodent Tslpr gene that encodes at least a substantial portion of the ectodomain of the rodent Tslpr protein. In some embodiments, the rodent is a mouse, and the mouse Tslpr genomic fragment being replaced encodes at least a substantial portion of the ectodomain of the endogenous mouse Tslpr protein. For example, the ectodomain of a mouse Tslpr of SEQ ID NO: 21 is defined by amino acids 20-243, examples of a substantial portion of the ectodomain can include amino acids 21-243, 22-243, 23-243, 24-243, 25-243, 26-243, 27-243, 20-241, 20-240, 20-239, and 20-238 of SEQ ID NO: 21. In some embodiments, a substantial portion of the ectodomain of a mouse Tslpr protein comprises amino acids 27-243 of SEQ ID: 21. In some embodiments, a substantial portion of the ectodomain of a mouse Tslpr protein comprises amino acids 25-243 of SEQ ID: 21. In some embodiments, a substantial portion of the ectodomain of a mouse Tslpr protein comprises amino acids 20-240 of SEQ ID: 21. In some embodiments, a substantial portion of the ectodomain of a mouse Tslpr protein comprises amino acids 20-238 of SEQ ID: 21. In some embodiments, the mouse Tslpr genomic fragment being replaced comprises exon 2 through the codon in exon 6 coding for the last amino acid of the ectodomain.

In some embodiments, the nucleotide sequence of a human TSLPR gene that replaces a genomic fragment of a rodent Tslpr gene at an endogenous rodent Tslpr locus is a cDNA sequence. In some embodiments, the human TSLPR nucleotide sequence that replaces a genomic fragment of a rodent Tslpr gene at an endogenous rodent Tslpr locus is a genomic fragment of a human TSLPR gene. In some embodiments, a genomic fragment of a human TSLPR gene that replaces a genomic fragment of a rodent Tslpr gene at an endogenous rodent Tslpr locus includes exons, in full or in part, of a human TSLPR gene, that encode at least a substantial portion of the ectodomain of the human TSLPR protein. Examples of a substantial portion of the ectodomain of a human TSLPR have been described above, e.g., amino acids 23-231, 24-231, 25-231, 26-231, 27-231, 28-231, 23-230, 23-229, 23-228, 23-227, or 23-226 of SEQ ID NO: 23. In some embodiments, a substantial portion of the ectodomain of a human TSLPR comprises amino acids 25-231 of SEQ ID NO: 23. In some embodiments, a substantial portion of the ectodomain of a human TSLPR comprises amino acids 27-231 of SEQ ID NO: 23. In some embodiments, a substantial portion of the ectodomain of a human TSLPR comprises amino acids 23-228 of SEQ ID NO: 23. In some embodiments, a substantial portion of the ectodomain of a human TSLPR comprises amino acids 23-226 of SEQ ID NO: 23. In some embodiments, the human genomic fragment comprises human TSLPR exon 2 through the codon in exon 6 coding for the last amino acid of the human TSLPR ectodomain.

In some embodiments, the human TSLPR nucleotide sequence inserted into an endogenous rodent Tslpr locus is operably linked to a genomic sequence of a rodent Tslpr gene that encodes a polypeptide substantially identical to the transmembrane-cytoplasmic sequence of a rodent Tslpr protein (such as an endogenous rodent, e.g., mouse or rat, Tslpr protein). In embodiments where the rodent is a mouse, the genomic sequence of a mouse Tslpr gene comprises, in some embodiments, exon 6 from the codon coding for the first amino acid of the transmembrane domain through exon 8 of a mouse Tslpr gene (e.g., an endogenous mouse Tslpr gene).

In some embodiments, the human TSLPR nucleotide sequence inserted into an endogenous rodent Tslpr locus is operably linked to a genomic sequence of a rodent Tslpr gene that encodes a polypeptide substantially identical to the signal peptide of a rodent Tslpr protein (such as an endogenous rodent, e.g., mouse or rat, Tslpr protein). In embodiments wherein the rodent is a mouse, the genomic sequence of a mouse Tslpr gene comprises, in some embodiments, exon 1, and optionally intron 1 in full or in part, of a mouse Tslpr gene (e.g., an endogenous mouse Tslpr gene).

In some embodiments, the rodent is a mouse, and a genomic fragment of an endogenous mouse Tslpr gene at an endogenous mouse Tslpr locus comprising exon 2 through the codon in exon 6 coding for the last amino acid of the mouse Tslpr ectodomain has been replaced with a genomic fragment of a human TSLPR gene comprising exon 2 through the codon in exon 6 coding for the last amino acid of the human TSLPR ectodomain. In some embodiments, a humanized Tslpr gene is formed at the endogenous rodent Tslpr locus and comprises exon 1 of a mouse Tslpr gene, exon 2 through the codon in exon 6 coding for the last amino acid of a human TSLPR gene, and exon 6 beginning from the codon coding for the first amino acid of a mouse Tslpr transmembrane domain through exon 8 of a mouse Tslpr gene.

In some embodiments, a rodent provided herein is heterozygous for a humanized Tslpr gene in its genome. In some embodiments, a rodent provided herein is homozygous for a humanized Tslpr gene in its genome.

In some embodiments, a humanized Tslpr gene results in an expression of the encoded humanized Tslpr protein in a rodent. In some embodiments, a humanized Tslpr protein is expressed in cells and tissues in which a counterpart rodent Tslpr protein in a control rodent (e.g., a rodent without the humanized Tslpr gene) is typically expressed, for example, on dendritic cells, CD4+ T cells and group 2 innate lymphoid cells, as well as on non-immune cell types like epithelial, endothelial and smooth muscle cells.

In some embodiments, rodents disclosed herein are incapable of expressing a rodent Tslpr protein, e.g., as a result of inactivation (e.g., deletion in full or in part) or replacement (in full or in part) of the endogenous rodent Tslpr gene.

### IL7RA Humanization

TSLP acts through a heterodimer composed of a chain specific for TSLP (referred to as “TSLPR” or “Tslpr”) and the IL7 receptor a chain (“IL7RA” for human and “Il7ra” for non-human or humanized molecule). IL7RA contains a signal peptide, an extracellular domain (“ECD” or “ectodomain”), a transmembrane domain and an intracellular (cytoplasmic) domain.

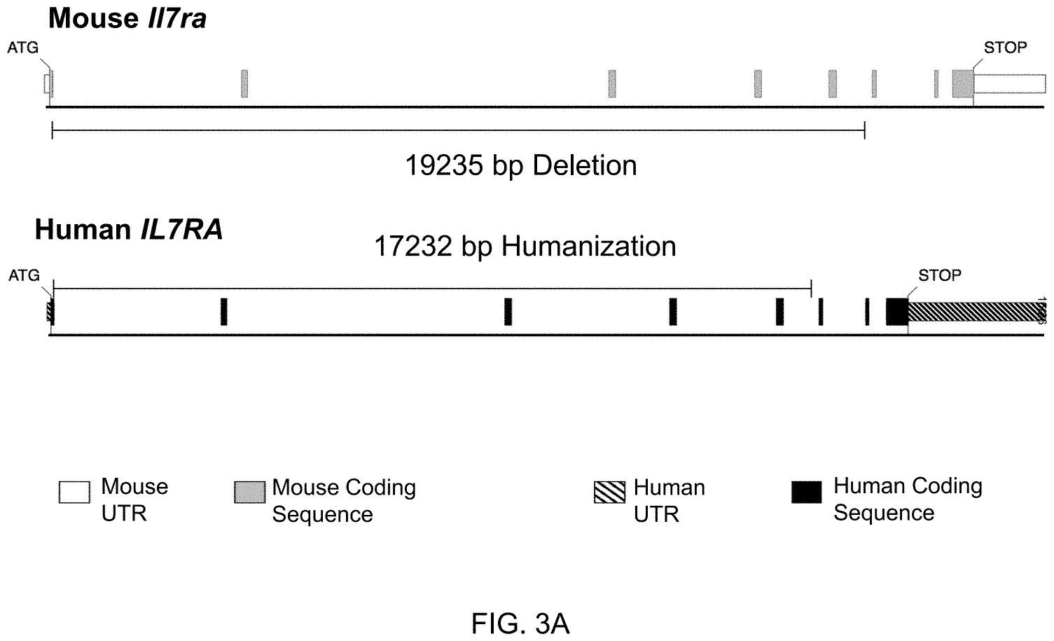

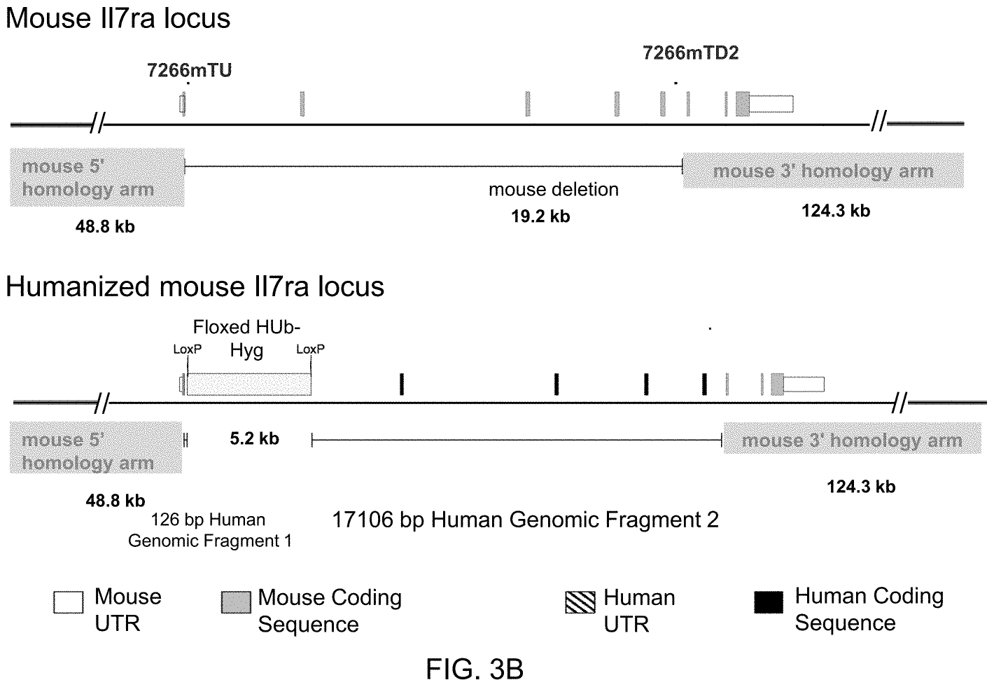

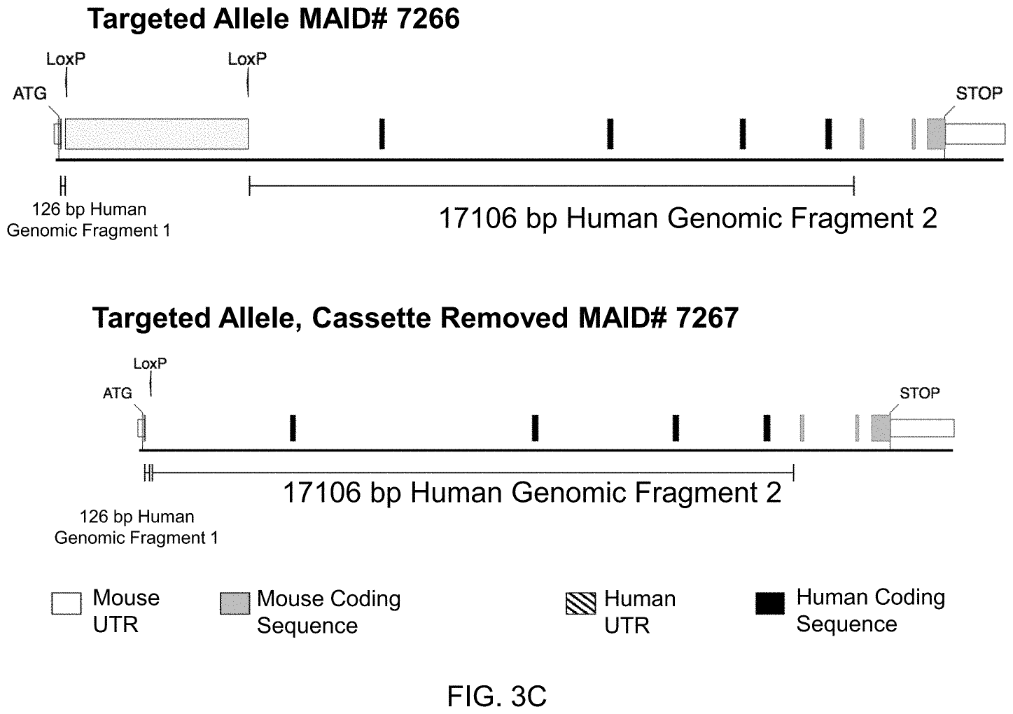

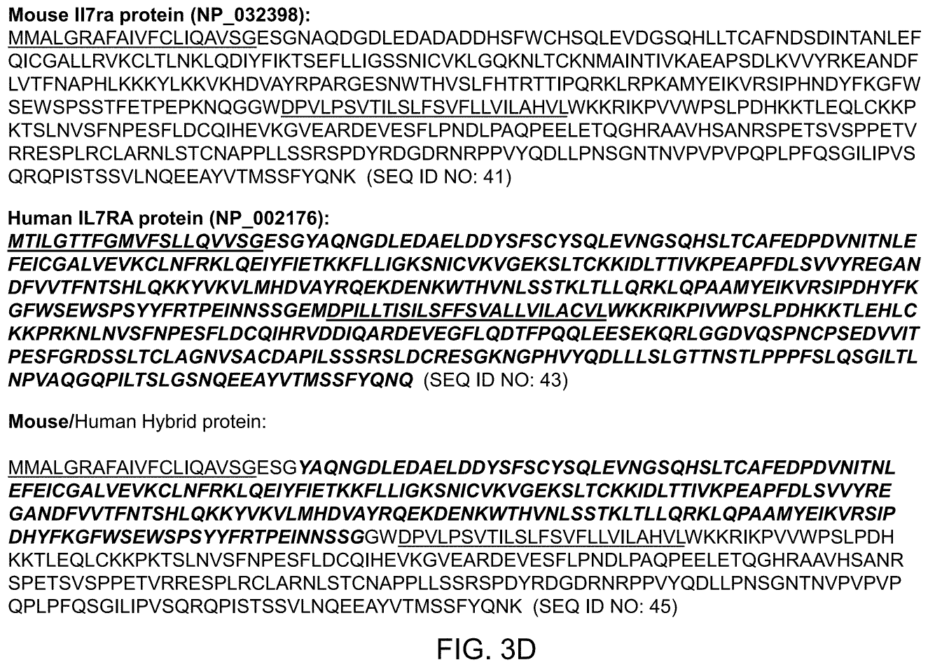

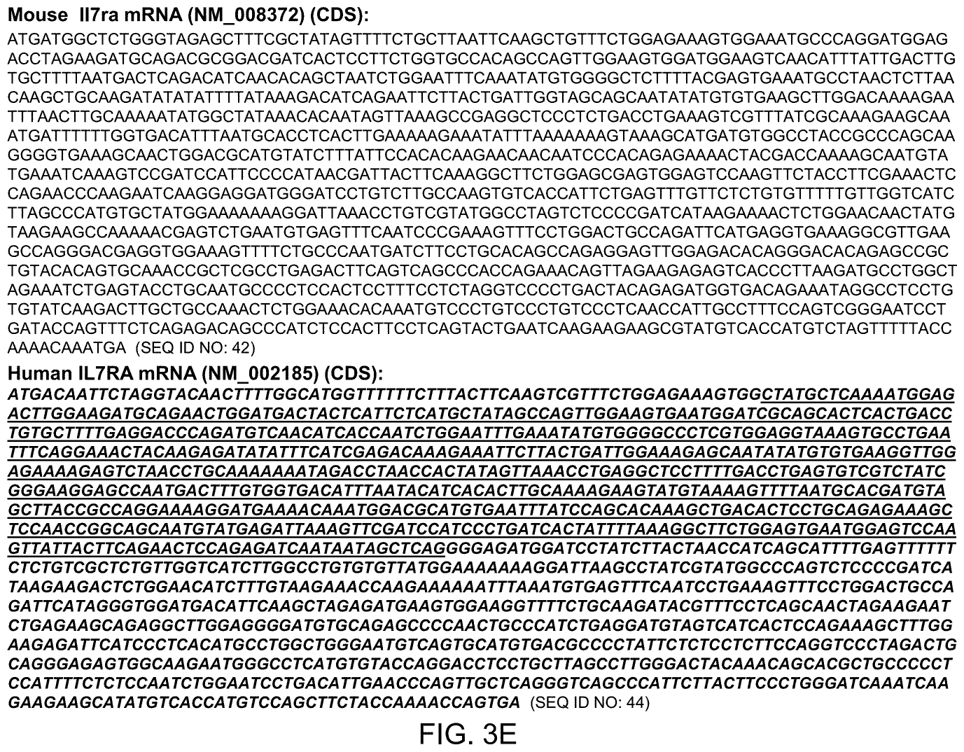

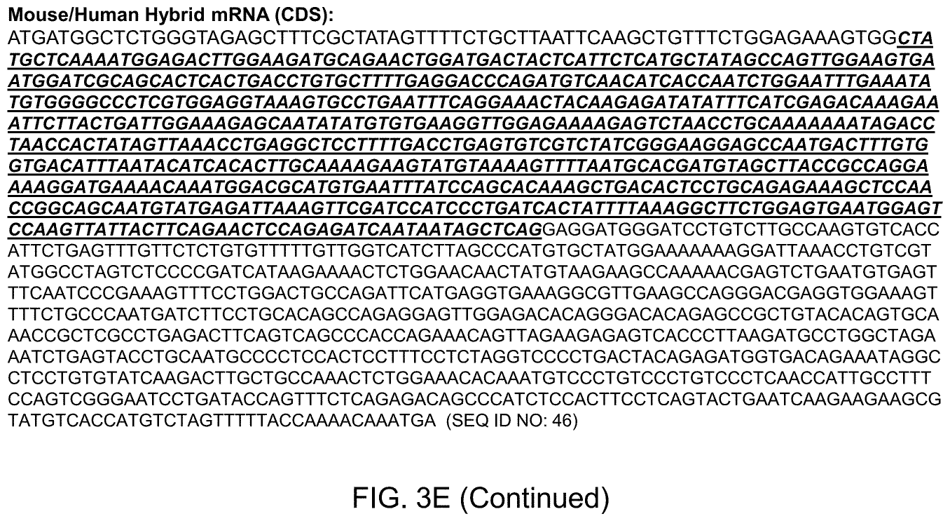

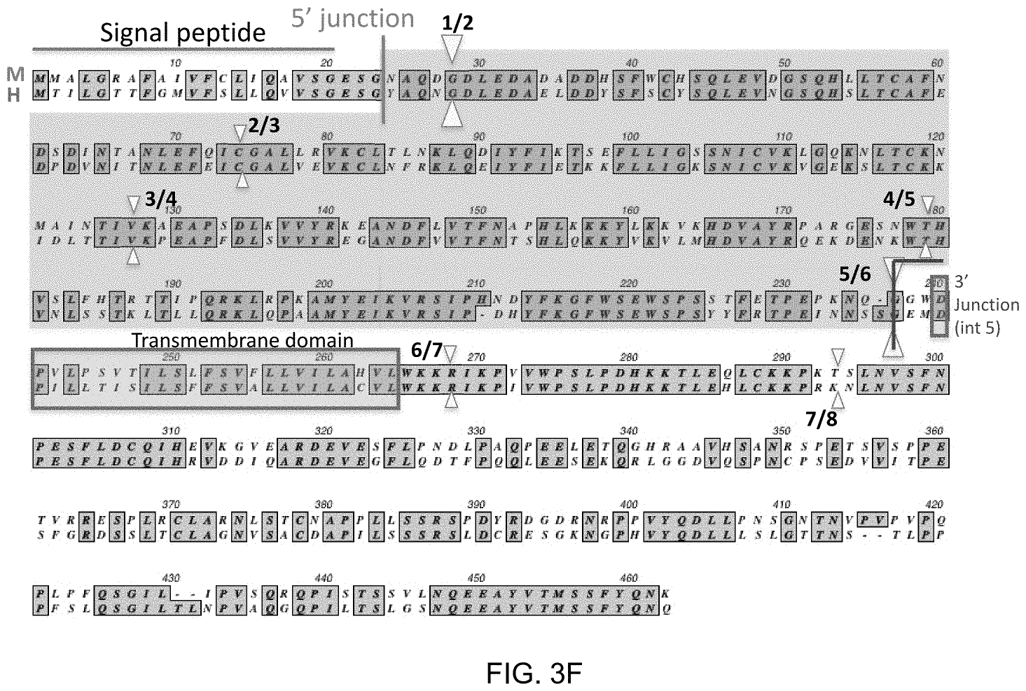

Exemplary sequences, including nucleic acid and protein sequences for human IL7RA, mouse Il7ra, rat Il7ra, and humanized (mouse-human hybrid) Il7ra, are disclosed in the Sequence Listing and summarized in Table 3. An alignment of human IL7RA and mouse Il7ra protein sequences is provided in FIG. 3F.

TABLE 3

| SEQ ID | | |
|---|---|---|
| NO | Description | Features |
| 41 | Mus musculus Il7ra Protein, | Length: 459 aa |
| | NP_032398 | Signal peptide: aa 1-20 |
| | | Ectodomain: aa 21-238 |
| | | Transmembrane: aa 239-263 |
| | | Intracellular: 264-459 |
| 42 | Mus musculus Il7ra mRNA | Length: 1380 bp |
| | (CDS), NM_008372 | |
| 43 | Homo sapiens IL7RA protein, | Length: 459 aa |
| | NP_002176 | Signal peptide: aa 1-20 |
| | | Ectodomain: aa 21-238 |
| | | Transmembrane: aa 239-263 |
| | | Intracellular: aa 264-459 |
| 44 | Homo sapiens IL7RA mRNA | Length: 1380 bp |
| | (CDS), NM_002185 | |
| 45 | Humanized mouse/human | Length: 459 aa |
| | chimeric Il7ra Protein | Signal peptide: 1-20 |
| | | (from mouse) |
| | | Ectodomain: aa 21-238 |
| | | (aa 21-236 from human |
| | | and aa 237-238 from mouse) |
| | | Transmembrane: aa 239-263 |
| | | (from mouse) |
| | | Intracellular: aa 264-459 |
| | | (from mouse) |
| 46 | Humanized mouse/human | Length: 1380 bp |
| | chimeric Il7ra mRNA (CDS) | |
| 47 | Rattus norvegicus Il7ra | Length: 457 aa |
| | Protein, NP_001099888 | |
| 48 | Rattus norvegicus Il7ra mRNA, | Length: 3124 bp |
| | NM_001106418.1 | |

In some embodiments, the rodents disclosed herein comprise a humanized Il7ra gene in the germline.

In some embodiments, a rodent disclosed herein comprises a humanized Il7ra gene in its genome that includes a nucleotide sequence of a rodent Il7ra gene (e.g., an endogenous rodent Il7ra gene) and a nucleotide sequence of a human IL7RA gene. As used herein, “a nucleotide sequence of a gene” includes a genomic sequence, an mRNA or cDNA sequence, in full or in part of the gene. As a non-limiting example, a nucleotide sequence of a human IL7RA gene includes a genomic sequence, an mRNA or cDNA sequence, in full or in part of the human IL7RA gene. The nucleotide sequence of the rodent Il7ra gene and the nucleotide sequence of the human IL7RA gene are operably linked to each other such that the humanized Il7ra gene in the rodent genome encodes a humanized Il7ra protein that has a Il7ra protein structure (comprising an ectodomain, a transmembrane domain and a cytoplasmic domain) and performs Il7ra functions (e.g., binds an 117 protein and forms a heterodimer with Tslpr which binds Tslp).

“Human IL7RA” gene and protein, as used herein, refers to IL7RA gene and protein of the human origin. In some embodiments, a human IL7RA protein comprises the the amino acid sequence of SEQ ID NO: 43. In some embodiments, a human IL7RA protein comprises an amino acid sequence at least 95% identical to the the amino acid sequence of SEQ ID NO: 43. In some embodiments, a human IL7RA protein comprises an amino acid sequence at least 98% identical to the the amino acid sequence of SEQ ID NO: 43. In some embodiments, a human IL7RA protein comprises an amino acid sequence at least 99% identical to the the amino acid sequence of SEQ ID NO: 43.

“Rodent Il7ra” gene and protein, as used herein, refers to Il7rar gene and protein of a rodent (e.g., mouse or rat) origin. In some embodiments, a mouse Il7ra protein comprises the the amino acid sequence of SEQ ID NO: 41. In some embodiments, a mouse Il7ra protein comprises an amino acid sequence at least 95% identical to the the amino acid sequence of SEQ ID NO: 41. In some embodiments, a mouse Il7rar protein comprises an amino acid sequence at least 98% identical to the the amino acid sequence of SEQ ID NO: 41. In some embodiments, a mouse Il7rar protein comprises an amino acid sequence at least 99% identical to the the amino acid sequence of SEQ ID NO: 41. In some embodiments, a rat Il7ra protein comprises the the amino acid sequence of SEQ ID NO: 47. In some embodiments, a rat Il7ra protein comprises an amino acid sequence at least 95% identical to the the amino acid sequence of SEQ ID NO: 47. In some embodiments, a rat Il7ra protein comprises an amino acid sequence at least 98% identical to the the amino acid sequence of SEQ ID NO: 47. In some embodiments, a rat Il7ra protein comprises an amino acid sequence at least 99% identical to the the amino acid sequence of SEQ ID NO: 47.

In some embodiments, a genetically modified rodent contains a humanized Il7ra gene in its genome, wherein the humanized Il7ra gene encodes a humanized Il7ra protein that contains an ectodomain that is substantially identical with the ectodomain of a human IL7RA protein. In some embodiments, an ectodomain that is substantially identical with the ectodomain of a human IL7RA protein exhibits the same functionality (e.g., ligand binding properties) as the ectodomain of a human IL7RA protein. An ectodomain or polypeptide that is “substantially identical with the ectodomain of a human IL7RA protein” can be a polypeptide that is at least 95% identical in sequence with the ectodomain of a human IL7RA protein; a polypeptide that is at least 98% identical in sequence with the ectodomain of a human IL7RA protein; or a polypeptide that is at least 99% identical in sequence with the ectodomain of a human IL7RA protein. An ectodomain or polypeptide that is “substantially identical with the ectodomain of a human IL7RA protein” may be a polypeptide that is 100% identical in sequence with the ectodomain of a human IL7RA protein. Alternatively or additionally, an ectodomain or polypeptide that is “substantially identical with the ectodomain of a human IL7RA protein” can be (ii) a polypeptide that differs from the ectodomain of a human IL7RA protein by not more than 10 amino acids, a polypeptide that differs from the ectodomain of a human IL7RA protein by not more than 7 amino acids, a polypeptide that differs from the ectodomain of a human IL7RA protein by not more than 5 amino acids, a polypeptide that differs from the ectodomain of a human IL7RA protein by not more than 4 amino acids, a polypeptide that differs from the ectodomain of a human TSLPR protein by not more than 3 amino, a polypeptide that differs from the ectodomain of a human TSLPR protein by not more than 2 amino acids, or a polypeptide that differs from the ectodomain of a human TSLPR protein by not more than 1 amino acid. Alternatively or additionally, an ectodomain or polypeptide that is “substantially identical with the ectodomain of a human IL7RA protein” can be (iii) a polypeptide that differs from the ectodomain of a human IL7RA protein only at the N- or C-terminal portion of the ectodomain, e.g., by having addition, deletion and/or substitution of amino acids at the N- and/or C-terminal portion of the ectodomain (i.e., within 5-10 amino acids from the N or C terminus of the ectodomain). Alternatively or additionally, an ectodomain or polypeptide that is “substantially identical with the ectodomain of a human IL7RA protein” can be (iv) a polypeptide that has one or more of the features delineated in (i)-(iii) above, e.g., a polypeptide that is at least 95% identical in sequence with the ectodomain of a human IL7RA protein and differs from the ectodomain of the human IL7RA protein only at the N- or C-terminal portion of the ectodomain by not more than 5 amino acids; or a polypeptide that is at least 98% identical in sequence with the ectodomain of a human IL7RA protein and differs from the ectodomain of the human IL7RA protein only at the N- or C-terminal portion of the ectodomain by not more than 3 amino acids. In some embodiments, a human IL7RA protein comprises the amino acid sequence as set forth in SEQ ID NO: 43, and its ectodomain is defined by amino acids 21-238 of SEQ ID NO: 43. In some embodiments, the humanized Il7ra gene encodes a humanized Il7ra protein whose ectodomain is substantially identical with the ectodomain of the human IL7RA protein as set forth in SEQ ID NO: 43, i.e., substantially identical with amino acids 21-238 of SEQ ID NO: 43. In some embodiments, the humanized Il7ra protein comprises an ectodomain that comprises amino acids 21-238 of SEQ ID NO: 43. In some embodiments, the humanized Il7ra protein comprises an ectodomain that comprises amino acids 21-237 of SEQ ID NO: 43. In some embodiments, the humanized Il7ra protein comprises an ectodomain that comprises amino acids 21-236 of SEQ ID NO: 43. In some embodiments, the humanized Il7ra protein comprises an ectodomain that comprises amino acids 22-238 of SEQ ID NO: 43. In some embodiments, the humanized Il7ra protein comprises an ectodomain that comprises amino acids 24-238 of SEQ ID NO: 43.

In some embodiments, the humanized Il7ra gene encodes a humanized Il7ra protein having an ectodomain that comprises the ectodomain of a human IL7RA or a substantial portion thereof, followed by amino acids from the C-terminus of the ectodomain of a rodent Il7ra (e.g., an endogenous rodent Il7ra). A “substantial portion of the ectodomain” of a human IL7RA (or a rodent IL7ra) protein refers to a polypeptide that is nearly the full ectodomain of a human IL7RA (or a rodent IL7ra) protein. In some embodiments, a substantial proportion of an ectodomain includes at least 95% of the full length ectodomain sequence. In some embodiments, a substantial proportion of an ectodomain includes at least 98% of the full length ectodomain sequence. In some embodiments, a substantial proportion of an ectodomain differs from the ectodomain by lacking not more than 10 amino acids at the N- or C-terminus of the ectodomain. In some embodiments, a substantial proportion of an ectodomain differs from the ectodomain by lacking not more than 7 amino acids at the N- or C-terminus of the ectodomain. In some embodiments, a substantial proportion of an ectodomain differs from the ectodomain by lacking not more than 5 amino acids at the N- or C-terminus of the ectodomain. In some embodiments, a substantial proportion of an ectodomain differs from the ectodomain by lacking not more than 3 amino acids at the N- or C-terminus of the ectodomain. For example, the ectodomain of the human IL7RA protein as set forth in SEQ ID NO: 43 is defined by amino acids 21-238, and examples of a substantial portion of the ectodomain can include amino acids 22-238, 23-238, 24-238, 21-237, 21-236, 21-235, of the human IL7RA protein as set forth in SEQ ID NO: 43. In some embodiments, a substantial portion of the ectodomain of human IL7RA comprises amino acids 21-236 of SEQ ID NO: 43. In some embodiments, a substantial portion of the ectodomain of human IL7RA comprises amino acids 21-237 of SEQ ID NO: 43. In some embodiments, a substantial portion of the ectodomain of human IL7RA comprises amino acids 22-238 of SEQ ID NO: 43. In some embodiments, a substantial portion of the ectodomain of human IL7RA comprises amino acids 23-238 of SEQ ID NO: 43. In some embodiments, the ectodomain of a humanized Il7ra protein comprises a substantial portion of the ectodomain of a human IL7RA, followed by 1-3 amino acids from the C-terminus of the ectodomain of a rodent Il7ra (e.g., an endogenous rodent Il7ra). In some embodiments, the ectodomain of a humanized Il7ra protein comprises amino acids 21-236 of SEQ ID NO: 43, followed by 2 amino acids from the C-terminus of the ectodomain of a rodent Il7ra. In some embodiments, the humanized Il7ra gene encodes a humanized Il7ra protein that contains an ectodomain comprising amino acids 21-238 of SEQ ID NO: 45—this ectodomain comprises amino acids 21-236 of SEQ ID NO: 43 (human IL7RA) and the last 2 amino acids (“GW”) from the C-terminus of the ectodomain of mouse Il7ra, and differs from the ectodomain of the human IL7RA protein of SEQ ID NO: 43 in the two amino acids at the C-terminus of the ectodomain (with “GW” in humanized Il7ra of SEQ ID NO: 45, as opposed to “EM” in human IL7RA of SEQ ID NO: 43) and is otherwise identical to the human IL7RA ectodomain.

In some embodiments, the humanized Il7ra gene encodes a humanized Il7ra protein that contains a transmembrane-cytoplasmic sequence (i.e., a sequence that includes both the transmembrane domain and the cytoplasmic domain) that is substantially identical with the transmembrane-cytoplasmic sequence of a rodent Il7ra protein, e.g., an endogenous rodent Il7ra protein. In some embodiments, a transmembrane-cytoplasmic sequence that is substantially identical with the transmembrane-cytoplasmic sequence of an endogenous rodent Il7ra protein exhibits the same functionality (e.g., signal transduction and/or interaction with intracellular molecules) as the transmembrane-cytoplasmic sequence of a rodent Il7ra protein such as an endogenous rodent Il7ra protein. A transmembrane-cytoplasmic sequence or polypeptide that is “substantially identical with the transmembrane-cytoplasmic sequence of a rodent Tslpr protein” can be (i) a polypeptide that is at least 95% identical in sequence with the transmembrane-cytoplasmic sequence of a rodent Il7ra protein, or a polypeptide that is at least 98% identical in sequence with the transmembrane-cytoplasmic sequence of a rodent Il7ra protein; in some embodiments. A transmembrane-cytoplasmic sequence or polypeptide that is “substantially identical with the transmembrane-cytoplasmic sequence of a rodent Tslpr protein” can be a polypeptide that is identical in sequence with the transmembrane-cytoplasmic sequence of a rodent Il7ra protein. Alternatively or additionally, a transmembrane-cytoplasmic sequence or polypeptide that is “substantially identical with the transmembrane-cytoplasmic sequence of a rodent Tslpr protein” can be (ii) a polypeptide that differs from the transmembrane-cytoplasmic sequence of a rodent Il7ra protein by not more than 5 amino acids, a polypeptide that differs from the transmembrane-cytoplasmic sequence of a rodent Il7ra protein by not more than 4 amino acids, a polypeptide that differs from the transmembrane-cytoplasmic sequence of a rodent Il7ra protein by not more than 3 amino acids, a polypeptide that differs from the transmembrane-cytoplasmic sequence of a rodent Il7ra protein by not more than 2 amino acids, a polypeptide that differs from the transmembrane-cytoplasmic sequence of a rodent Il7ra protein by not more than 1 amino acid. Alternatively or additionally, a transmembrane-cytoplasmic sequence or polypeptide that is “substantially identical with the transmembrane-cytoplasmic sequence of a rodent Tslpr protein” can be (iii), a polypeptide that differs from the transmembrane-cytoplasmic sequence of a rodent Il7ra protein only at the N- or C-terminus, e.g., by having addition, deletion or substitution of amino acids at the N- or C-terminal portion of the transmembrane-cytoplasmic sequence. Alternatively or additionally, a transmembrane-cytoplasmic sequence or polypeptide that is “substantially identical with the transmembrane-cytoplasmic sequence of a rodent Tslpr protein” can be (iv) a polypeptide having one or more features delienated in (i)-(iii) above, e.g., a polypeptide that is at least 95% identical in sequence with the transmembrane-cytoplasmic sequence of a rodent Il7ra protein, and differs from the transmembrane-cytoplasmic sequence of a rodent Il7ra protein only at the N- or C-terminus by not more than 3 amino acids. By “the N- or C-terminal portion of the transmembrane-cytoplasmic sequence” is meant within 5-10 amino acids from the N-terminus of the transmembrane domain or within 5-10 amino acids from the C-terminus of the cytoplasmic domain. In some embodiments, a humanized Il7ra protein contains a transmembrane-cytoplasmic sequence that is substantially identical with the transmembrane-cytoplasmic sequence of a mouse Il7ra protein (such as an endogenous mouse Il7ra protein), or with the transmembrane-cytoplasmic sequence of a rat Il7ra protein (such as an endogenous rat Il7ra protein).

In some embodiments, the humanized Il7ra gene encodes a humanized Il7ra protein that contains a signal peptide that is substantially identical with the signal peptide of a rodent Il7ra protein (e.g., an endogenous rodent Il7ra protein). In some embodiments, the humanized Il7ra gene encodes a humanized Il7ra protein that contains a signal peptide that is substantially identical with the signal peptide of a human IL7RA protein (e.g., the human IL7RA protein as set forth in SEQ ID NO: 43). A signal peptide that is “substantially identical” with the signal peptide of a reference protein (either a human IL7RA protein or a rodent Il7ra protein), can be (i) a polypeptide that is at least 95% identical in sequence with the signal peptide of the reference protein, or a polypeptide that is identical in sequence with the signal peptide of the reference protein. Alternatively or additionally, a signal peptide that is “substantially identical” with the signal peptide of a reference protein can be (ii) a polypeptide that differs from the signal peptide of the reference protein by not more than 3 amino acids, a polypeptide that differs from the signal peptide of the reference protein by not more than 2 amino acids; in some embodiments, or a polypeptide that differs from the signal peptide of the reference protein by not more than 1 amino acid. Alternatively or additionally, a signal peptide that is “substantially identical” with the signal peptide of a reference protein can be (iii) a polypeptide that differs from the signal peptide of the reference protein only at the N- or C-terminus, e.g., by having addition, deletion or substitution of amino acids at the N- or C-terminal portion of the signal peptide; or (iv) a polypeptide having one or more features delineated in (i)-(iii) above, e.g., a polypeptide that is at least 95% identical with the signal peptide of a reference protein and differs from the signal peptide of a reference protein only at the N- or C-terminus by not more than 3 amino acids. By “the N- or C-terminal portion of the signal peptide” is meant within 5 amino acids from the N- or C-terminus of the signal peptide. In some embodiments, a humanized Il7ra protein includes a signal peptide substantially identical with the signal peptide of a mouse Il7ra protein (such as an endogenous mouse Il7ra protein). In some embodiments, a humanized Il7ra protein includes a signal peptide substantially identical with the signal peptide of a rat Il7ra protein (such as an endogenous rat Il7ra protein). In some embodiments, a humanized Il7ra protein includes a signal peptide substantially identical with the signal peptide of a human IL7RA protein (e.g., the human IL7RA protein as set forth in SEQ ID NO: 43).

In some embodiments, the humanized Il7ra gene in the genome of a genetically modified rodent includes a nucleotide sequence of a human IL7RA gene (“a human IL7RA nucleotide sequence”) and a nucleotide sequence of a rodent Il7ra gene (“a rodent Il7ra nucleotide sequence”, such as an endogenous roden Il7ra nucleotide sequence), wherein the human IL7RA nucleotide sequence encodes at least a substantial portion of the ectodomain of a human IL7RA protein. In some embodiments, the human IL7RA nucleotide sequence is a cDNA sequence. In some embodiments, the human IL7RA nucleotide sequence in a humanized Il7ra gene encodes the ectodomain of a human IL7RA protein (e.g., the human IL7RA protein of SEQ ID NO: 43). In some embodiments, the human IL7RA nucleotide sequence is a genomic fragment of a human IL7RA gene. In some embodiments, the human IL7RA nucleotide sequence is a genomic fragment of a human IL7RA gene comprising from the codon in exon 2 that encodes the first amino acid in the mature protein sequence through exon 5 (i.e., through the codon that encodes the amino acid residue that is two amino acids before the start of the transmembrane segment of the human IL7RA protein. In some embodiments, the human IL7RA genomic fragment encodes amino acids 21-238 of a human IL7RA protein (e.g, the human IL7RA protein as set forth in SEQ ID NO: 43). In some embodiments, the human IL7RA genomic fragment encodes amino acids 21-237 of a human IL7RA protein (e.g, the human IL7RA protein as set forth in SEQ ID NO: 43). In some embodiments, the human IL7RA genomic fragment encodes amino acids 21-236 of a human IL7RA protein (e.g, the human IL7RA protein as set forth in SEQ ID NO: 43). In some embodiments, the human IL7RA genomic fragment encodes amino acids 21-235 of a human IL7RA protein (e.g, the human IL7RA protein as set forth in SEQ ID NO: 43). In some embodiments, the human IL7RA genomic fragment encodes amino acids 22-238 of a human IL7RA protein (e.g, the human IL7RA protein as set forth in SEQ ID NO: 43). In some embodiments, the human IL7RA genomic fragment encodes amino acids 24-238 of a human IL7RA protein (e.g, the human IL7RA protein as set forth in SEQ ID NO: 43). In some embodiments, the human IL7RA genomic fragment further comprises a 5′ portion of intron 5.

In some embodiments, the humanized Il7ra gene in the genome of a genetically modified rodent includes a rodent Il7ra nucleotide sequence and a human IL7RA nucleotide sequence, wherein the rodent Il7ra nucleotide sequence encodes a polypeptide substantially identical to the transmembrane-cytoplasmic sequence of a rodent Il7ra protein (e.g., an endogenous rodent Il7ra protein). In some embodiments, the rodent Il7ra nucleotide sequence present in a humanized Il7ra gene encodes the transmembrane-cytoplasmic sequence of an endogenous rodent Il7ra protein. In some embodiments, the rodent Il7ra nucleotide sequence present in a humanized Il7ra gene is a mouse Il7ra nucleotide sequence; and in some such embodiments, the mouse Il7ra nucleotide sequence comprises a 3′ portion of intron 5 and exon 6 through exon 8 of a mouse Il7ra gene (e.g., an endogenous mouse Il7ra gene).

In some embodiments, the humanized Il7ra gene in the genome of a genetically modified rodent includes a rodent Il7ra nucleotide sequence upstream (5′) of a human IL7RA nucleotide sequence, wherein the rodent Il7ra nucleotide sequence encodes a polypeptide substantially identical to the signal peptide of a rodent Il7ra protein (e.g., an endogenous rodent Il7ra protein). In some embodiments, the rodent Il7ra nucleotide sequence encoding a polypeptide substantially identical to the signal peptide of a rodent Il7ra protein is a mouse Il7ra nucleotide sequence (e.g., an endogenous mouse Il7ra nucleotide sequence), or a rat Il7ra nucleotide sequence (e.g., an endogenous rat Il7ra nucleotide sequence). In some embodiments, a mouse Il7ra nucleotide sequence in a humanized Il7ra gene comprises the portion of exon 1 of a mouse Il7ra gene (e.g., an endogenous mouse Il7ra gene) that codes for the signal peptide of the mouse Il7ra; in some embodiments, the mouse Il7ra nucleotide sequence also comprises the 5′ UTR of exon 1 of the mouse Il7ra gene.

In some embodiments, the humanized Il7ra gene is operably linked to rodent Il7ra 5′ regulatory sequences such as endogenous rodent Il7ra regulatory sequences, e.g., a 5′ transcriptional regulatory sequence(s) such as promoter and/or enhancers, such that expression of the humanized Il7ra gene is under control of the rodent Il7ra 5′ regulatory sequence(s).

In some embodiments, the humanized Il7ra gene is at an endogenous rodent Il7ra locus. In some embodiments, the humanized Il7ra gene is at a locus other than an endogenous rodent Il7ra locus; e.g., as a result of random integration. In some embodiments where the humanized Il7ra gene is at a locus other than an endogenous rodent Il7ra locus, the rodents are incapable of expressing a rodent Il7ra protein, e.g., as a result of inactivation (e.g., deletion in full or in part) of the endogenous rodent Il7ra gene.

In some embodiments where a humanized Il7ra gene is at an endogenous rodent Il7ra locus, the humanized Il7ra gene results from a replacement of a nucleotide sequence of an endogenous rodent Il7ra gene at the endogenous rodent Il7ra locus with a nucleotide sequence of a human IL7RA gene.

In some embodiments, the nucleotide sequence of an endogenous rodent Il7ra gene at an endogenous rodent Il7ra locus that is being replaced is a genomic fragment of an endogenous rodent Il7ra gene that encodes at least a substantial portion of the ectodomain of the rodent Il7ra protein. In some embodiments, the rodent is a mouse, and the mouse Il7ra genomic fragment being replaced encodes at least a substantial portion of the ectodomain of the endogenous mouse Il7ra protein. Examples of a substantial portion of the ectodomain of the endogenous mouse Il7ra protein include amino acids 22-238, 23-238, 24-238, 21-237, 21-236, or 21-235 of the endogenous mouse Il7ra protein (e.g., SEQ ID NO: 41). In some embodiments, the mouse Il7ra genomic fragment being replaced encodes amino acids 21-235 of of the endogenous mouse Il7ra protein (e.g., SEQ ID NO: 41). In some embodiments, the mouse Il7ra genomic fragment being replaced encodes amino acids 21-236 of of the endogenous mouse Il7ra protein (e.g., SEQ ID NO: 41). In some embodiments, the mouse Il7ra genomic fragment being replaced encodes amino acids 21-237 of of the endogenous mouse Il7ra protein (e.g., SEQ ID NO: 41). In some embodiments, the mouse Il7ra genomic fragment being replaced encodes amino acids 21-238 of of the endogenous mouse Il7ra protein (e.g., SEQ ID NO: 41). In some embodiments, the mouse Il7ra genomic fragment being replaced encodes amino acids 22-238 of of the endogenous mouse Il7ra protein (e.g., SEQ ID NO: 41). In some embodiments, the mouse Il7ra genomic fragment being replaced encodes amino acids 23-238 of of the endogenous mouse Il7ra protein (e.g., SEQ ID NO: 41). In some embodiments, the mouse Il7ra genomic fragment being replaced comprises from the codon in exon 1 that encodes the first amino acid of the mature Il7ra protein through exon 5, and in some embodiments, through a 5′ portion of intron 5, of the the mouse Il7ra gene.

In some embodiments, the nucleotide sequence of a human IL7RA gene that replaces a genomic fragment of a rodent Il7ra gene at an endogenous rodent Il7ra locus is a cDNA sequence. In some embodiments, the human IL7RA nucleotide sequence that replaces a genomic fragment of a rodent Il7ra gene at an endogenous rodent Il7ra locus is a genomic fragment of a human IL7RA gene. In some embodiments, a genomic fragment of a human IL7RA gene that replaces a genomic fragment of a rodent Il7ra gene at an endogenous rodent Il7ra locus includes exons, in full or in part, of a human IL7RA gene, that encode at least a substantial portion of the ectodomain of the human IL7RA protein. In some embodiments, the human IL7RA genomic fragment encodes amino acids 21-238 of a human IL7RA, e.g., the human IL7RA as set forth in SEQ ID NO: 43. In some embodiments, the human IL7RA genomic fragment encodes amino acids 21-237 of a human IL7RA, e.g., the human IL7RA as set forth in SEQ ID NO: 43. In some embodiments, the human IL7RA genomic fragment encodes amino acids 21-236 of a human IL7RA, e.g., the human IL7RA as set forth in SEQ ID NO: 43. In some embodiments, the human IL7RA genomic fragment encodes amino acids 21-235 of a human IL7RA, e.g., the human IL7RA as set forth in SEQ ID NO: 43. In some embodiments, the human IL7RA genomic fragment encodes amino acids 22-238 of a human IL7RA, e.g., the human IL7RA as set forth in SEQ ID NO: 43. In some embodiments, the human IL7RA genomic fragment encodes amino acids 24-238 of a human IL7RA, e.g., the human IL7RA as set forth in SEQ ID NO: 43. In some embodiments, the human genomic fragment comprises the codon in exon 1 that encodes the first amino acid of the mature human IL7RA protein through exon 5, and in some embodiments, through a 5′ portion of intron 5 of a human IL7RA gene.

In some embodiments, the human IL7RA nucleotide sequence inserted into an endogenous rodent Il7ra locus is operably linked to a genomic sequence of a rodent Il7ra gene that encodes a polypeptide substantially identical to the transmembrane-cytoplasmic sequence of a rodent Il7ra protein (such as an endogenous rodent, e.g., mouse or rat, Il7ra protein). In embodiments where the rodent is a mouse, the genomic sequence of a mouse Il7ra gene comprises, in some embodiments, exon 6 through exon 8 of a mouse Il7ra gene (e.g., an endogenous mouse Il7ra gene); in some embodiments, a 3′ portion of intron 5 through exon 8 of a mouse Il7ra gene (e.g., an endogenous mouse Il7ra gene).

In some embodiments, the human IL7RA nucleotide sequence inserted into an endogenous rodent Il7ra locus is operably linked to a genomic sequence of a rodent Il7ra gene that encodes a polypeptide substantially identical to the signal peptide of a rodent Il7ra protein (such as an endogenous rodent, e.g., mouse or rat, Il7ra protein). In embodiments wherein the rodent is a mouse, the genomic sequence of a mouse Il7ra gene comprises, in some embodiments, the portion of exon 1 of a mouse Il7ra gene (e.g., an endogenous mouse Il7ra gene) that codes for the signal peptide of mouse Il7ra; and in some embodiments, the 5′ UTR of exon 1 and the portion of exon 1 of a mouse Il7ra gene (e.g., an endogenous mouse Il7ra gene) that codes for the signal peptide of mouse Il7ra.

In some embodiments, the rodent is a mouse, and a genomic fragment of an endogenous mouse Il7ra gene at an endogenous mouse Il7ra locus comprising from the first codon in exon 1 coding for the first mature Il7ra amino acid through exon 5 (or in some embodiments, through a 5′ portion of intron 5) has been replaced with a genomic fragment of a human IL7RA gene comprising from the first codon in exon 1 coding for the first mature IL7RA amino acid through exon 5 (or in some embodiments, through a 5′ portion of intron 5). In some embodiments, a humanized Il7ra gene is formed at the endogenous rodent Il7ra locus and comprises 5′UTR and the signal-peptide coding portion of exon 1 of a mouse Il7ra gene, the mature amino acids coding portion of exon 1 through exon 5 of a human IL7RA gene, and exon 6 through exon 8 of a mouse Il7ra gene; and in some such embodiments, intron 5 of the humanized Il7ra gene includes a 5′ portion from human intron 5 and a 3′ portion from the endogenous mouse intron 5.

In some embodiments, the rodent is a mouse comprising a humanized Il7ra gene at the endogenous mouse Il7ra locus, wherein the humanized Il7ra gene encodes a humanized Il7ra protein that comprises a signal peptide at least substantially identical to the signal peptide of a human IL7RA protein, an ectodomain at least substantially identical to the ectodomain of the human IL7RA protein, and the transmembrane-cytoplasmic domains of the endogenous mouse Il7ra protein. In some embodiments, the ectodomain of a humanized Il7ra protein comprises the full length ectodomain of a human IL7RA protein. In some embodiments, the ectodomain of a humanized Il7ra protein comprises (i) nearly the full length ectodomain of a human IL7RA protein except for 2 amino acids at the C-terminus of the ectodomain of the human IL7RA protein, and (ii) 2 amino acids at the C-terminus of the ectodomain of the endogenous mouse Il7ra protein. In some embodiments, the rodent is a mouse comprising a humanized Il7ra gene at the endogenous mouse Il7ra locus as described in CN111808882A, incorporated herein by reference in its entirety.

In some embodiments, a rodent provided herein is heterozygous for a humanized Il7ra gene in its genome. In some embodiments, a rodent provided herein is homozygous for a humanized Il7ra gene in its genome.

In some embodiments, a humanized Il7ra gene results in an expression of the encoded humanized Il7ra protein in a rodent. In some embodiments, a humanized Il7ra protein is expressed in cells and tissues in which a counterpart rodent Il7ra protein in a control rodent (e.g., a rodent without the humanized Il7ra gene) is typically expressed, for example, on T-lymphocytes.

In some embodiments, rodents disclosed herein are incapable of expressing a rodent Il7ra protein, e.g., as a result of inactivation (e.g., deletion in full or in part) or replacement (in full or in part) of the endogenous rodent Il7ra gene.

### Additional Genetic Features

In some embodiments, rodents disclosed herein further comprise a humanized Sirpα gene in their genome. Humanization of a rodent Sirpα gene has been described in, e.g., WO 2015/042557 A1 (Regeneron Pharmaceuticals Inc.) and US20190373867A1 (Beijing Biocytogen), incorporated herein by reference in their entireties.

In some embodiments, the humanized Sirpα gene encodes a humanized Sirpα protein comprising the extracellular domain, in full or in part, of a human SIRPα protein. In some embodiments, the humanized Sirpα gene encodes a humanized Sirpα protein comprising an extracellular portion of a human SIRPα protein responsible for ligand binding (i.e., binding to CD47). In some embodiments, the humanized Sirpα gene encodes a humanized Sirpα protein comprising amino acid residues 28-362 of a human SIRPα protein, e.g., the human SIRPα protein as set forth in GenBank Accession No. NP 001035111.1. In some embodiments, the humanized Sirpα gene encodes a humanized Sirpα protein comprising the transmembrane and cytoplasmic domains of a rodent Sirpα protein (e.g., an endogenous rodent Sirpα protein). In some embodiments, a humanized Sirpα gene comprises exons 2, 3, and 4 of a human SIRPα gene. In some embodiments, a humanized Sirpα gene is located at an endogenous rodent Sirpα locus. In some embodiments, a humanized Sirpα gene is formed as a result of a replacement of exons 2-4 of an endogenous rodent Sirpα gene at an endogenous rodent Sirpα locus by exons 2-4 of a human SIRPα gene. In some embodiments, a humanized Sirpα gene is located at an endogenous rodent Sirpα locus and comprises exon 1 of the endogenous rodent Sirpα gene, exons 2-4 of a human SIRPα gene, and exons 5-8 of the endogenous rodent Sirpα gene, wherein the humanized Sirpα gene is operably linked to the rodent Sirpα promoter at the endogenous rodent Sirpα locus. In some embodiments, a rodent is heterozygous for a humanized Sirpα gene. In some embodiments, a rodent is homozygous for a humanized Sirpα gene. In some embodiments, a rodent comprising a humanized Sirpα gene expresses a humanized Sirpα protein, such as a protein comprising the ectodomain of a human SIRPα protein and the transmembrane-cytoplasmic domains of a rodent Sirpα protein. In some embodiments, rodents disclosed herein are incapable of expressing an endogenous rodent Sirpα protein (e.g., as a result of disruption or replacement of an endogenous rodent Sirpα gene).

In some embodiments, rodents disclosed herein further comprise in their genome a humanized Tpo (thrombopoietin) gene. Humanization of a rodent Tpo gene has been described in, e.g., U.S. Pat. No. 8,541,646 (Regeneron Pharmaceuticals Inc., Yale University, and Institute for Research in Biomedicine IRB), and Rongvaux et al. (Proc Natl Acad Sci USA. 2011; 108(6): 2378-2383), incorporated herein by reference in their entireties. In some embodiments, the humanization comprises replacement of an endogenous rodent Tpo gene with a human TPO gene. In some embodiments, a rodent expresses a human TPO protein from a humanized Tpo gene. In some embodiments, a rodent is heterozygous for a humanized Tpo gene. In some embodiments, a rodent is homozygous for a humanized Tpo gene. In some embodiments, a rodent comprising a humanized Tpo gene is incapable of expressing an endogenous rodent Tpo protein (e.g., as a result of disruption or replacement of an endogenous rodent Tpo gene).

In some embodiments, rodents disclosed herein further comprise in their genome a humanized GM-CSF/IL-3 locus, where an endogenous rodent GM-CSF gene is replaced with a human GM-CSF gene and an endogenous rodent IL-3 gene has been replaced with a human IL-3 gene. Humanization of a rodent GM-CSF/IL-3 locus has been described in, e.g., U.S. Pat. No. 8,541,646 (Regeneron Pharmaceuticals Inc., Yale University, and Institute for Research in Biomedicine IRB) and Willinger et al. (PNAS, 108(6):2390-2395, 2011), both incorporated herein in their entireties. In some embodiments, a rodent is heterozygous for a humanized GM-CSF/IL-3 locus. In some embodiments, a rodent is homozygous for a humanized GM-CSF/IL-3 locus. In some embodiments, a rodent expresses human GM-CSF and human IL-3 from a humanized GM-CSF/IL-3 locus. In some embodiments, rodents disclosed herein are incapable of expressing an endogenous rodent GM-CSF protein and incapable of expressing an endogenous rodent IL-3 protein (e.g., as a result of disruption or replacement of an endogenous rodent GM-CSF/IL-3 locus).

In some embodiments, rodents disclosed herein have their endogenous RAG2 gene disrupted; and in some embodiments, a rodent is homozygous for the disruption (RAG2−/− or RAG knockout) and is incapable of expressing an endogenous RAG2 protein. In some embodiments, rodents disclosed herein have their endogenous IL-2RG gene disrupted; and in some embodiments, a rodent is homozygous for the disruption (IL-2RG−/ or IL-2RG knockout) and is incapable of expressing an endogenous IL-2RG protein (also known as “γc”). RAG2 and IL-2RG double knock-out (DKO) rodents are known immunodeficient rodents (see, e.g., Traggiai E et al. (2004) Development of a human adaptive immune system in cord blood cell-transplanted mice, Science 304:104-107, incorporated herein by reference in its entirety) and readily available commercially (e.g., from Taconic Biosciences, Inc., New York).

In some embodiments, rodents disclosed herein comprise in their genome a humanized Tslp gene and a humanized Sirpα gene, and are homozygous null for both RAG2 and IL-2RG genes. In some such embodiments, a rodent further comprises in its genome a humanized Tpo gene, and/or a humanized GM-CSF/IL-3 locus. A rodent can be heterozygous or homozygous for a humanized gene.

In some embodiments, rodents disclosed herein comprise in their genome a humanized Tslp gene, a humanized Tslpr gene, and a humanized Sirpα gene, and are homozygous null for both RAG2 and IL-2RG genes. In some such embodiments, a rodent further comprises in its genome a humanized Tpo gene and/or a humanized GM-CSF/IL-3 locus. Rodents can be homozygous or heterozygous for a humanized gene.

In some embodiments, rodents disclosed herein comprise in their genome a humanized Tslp gene, a humanized Tslpr gene, a humanized Il7ra gene, and a humanized Sirpα gene, and are homozygous null for both RAG2 and IL-2RG genes. In some such embodiments, a rodent further comprises in its genome a humanized Tpo gene, and/or a humanized GM-CSF/IL-3 locus. Rodents can be homozygous or heterozygous for a humanized gene.

Rodent Species and Strains

In some embodiments, rodents of this disclosure include, as non-limiting examples, a mouse, a rat, and a hamster. In some embodiments, a rodent is selected from the superfamily Muroidea. In some embodiments, a rodent of this disclosure is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, Malagasy rats and mice), Platacanthomyidae (e.g., spiny dormice), and Spalacidae (e.g., mole rates, bamboo rats, and zokors). In some embodiments, a rodent of this disclosure is selected from a true mouse or rat (family Muridae), a gerbil, a spiny mouse, and a crested rat. In some embodiments, a mouse of this disclosure is from a member of the family Muridae.

In some embodiments, a rodent is a mouse. In some embodiments, the rodent is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, a rodent is a mouse of a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129/SvJae, 129S6 (129/SvEvTac), 129S7, 129S8, 129T1, 129T2 (see, e.g., Festing et al., 1999, Mammalian Genome 10:836; Auerbach et al., 2000, Biotechniques 29(5):1024-1028, 1030, 1032). In some embodiments, a rodent is a mouse that is a mix of a 129 strain and a C57BL/6 strain. In some embodiments, a rodent is a mouse that is a mix of aforementioned 129 strains, or a mix of aforementioned BL/6 strains. In some embodiments, a rodent is a mouse of a BALB strain, e.g., BALB/c strain. In some embodiments, a rodent is a mouse that is a mix of a BALB strain and another aforementioned strain.

In some embodiments, a rodent is a rat. In some certain embodiments, a rat is selected from a Wistar rat, an LEA strain, a Sprague Dawley strain, a Fischer strain, F344, F6, and Dark Agouti. In some embodiments, a rat strain as described herein is a mix of two or more strains selected from the group consisting of Wistar, LEA, Sprague Dawley, Fischer, F344, F6, and Dark Agouti.

### Tissues and Cells of Genetically Modified Rodents

In some embodiments, disclosed herein is an isolated rodent cell or tissue whose genome comprises a humanized Tslp gene, a humanized Tslpr gene, a humanized Il7ra gene, or a combination thereof. In some embodiments, an isolated rodent cell or tissue further comprises one or more of the additional genetic modifications described above (e.g., a humanized Sirpα gene, RAG2−/− and IL-2RG−/−, a humanized Tpo gene, or a humanized GM-CSF/IL-3 locus).

In some embodiments, a tissue is selected from adipose, bladder, brain, breast, bone marrow, eye, heart, intestine, kidney, liver, lung, lymph node, muscle, pancreas, plasma, serum, skin, spleen, stomach, thymus, testis, ovum, and a combination thereof.

In some embodiments, a cell is selected from an epithelial cell, a kerotinocyte, a dendritic cell, lymphocyte (e.g., a B or T cell), macrophage, mast cell, and basophil. In some embodiments, an isolated rodent cell is a rodent embryonic stem cell. In some embodiments, an isolated rodent cell is a rodent egg, or a rodent sperm.

### Compositions and Methods for Making Humanized Rodents

Disclosed herein is a targeting vector (or nucleic acid construct) comprising a human TSLP nucleotide sequence, a human TSLPR nucleotide sequence, or a human IL7RA nucleotide sequence, desired to be integrated into a rodent locus to form a humanized gene as described herein.

In some embodiments, a targeting vector comprises a human TSLP nucleotide sequence which encodes at least a substantial portion of the mature protein sequence of a human TSLP protein as described hereinabove. In some embodiments, the human TSLP nucleotide sequence encodes a polypeptide comprising amino acids 29-159 of SEQ ID NO: 3. In some embodiments, the human TSLP nucleotide sequence comprises exon 1 beginning from the codon for the first amino acid of the mature protein through the STOP codon in exon 4 of a human TSLP gene.

In some embodiments, a targeting vector comprises a human TSLPR nucleotide sequence which encodes at least a substantial portion of the ectodomain of a human TSLPR protein as described hereinabove. In some embodiments, the human TSLPR nucleotide sequence encodes a polypeptide comprising amino acids 27-231 of SEQ ID NO: 23. In some embodiments, the human TSLPR nucleotide sequence comprises exon 2, through the codon in exon 6 encoding the last ectodomain amino acid, of a human TSLPR gene.

In some embodiments, a targeting vector comprises a human IL7RA nucleotide sequence which encodes at least a substantial portion of the ectodomain of a human IL7RA protein as described hereinabove. In some embodiments, the human IL7RA nucleotide sequence encodes a polypeptide comprising amino acids 21-236 of SEQ ID NO: 43. In some embodiments, the human IL7RA nucleotide sequence comprises from the codon in exon 1 that encodes the first amino acid of the mature IL7RA protein, through exon 5 (and in some embodiments, through a 5′ portion of intron 5) of a human IL7RA gene.

The targeting vector also includes 5′ and 3′ rodent sequences flanking the human nucleotide sequence to be integrated, also known as 5′ and 3′ homology arms, that mediate homologous recombination and integration of the human nucleotide sequence into the target rodent locus (e.g., an endogenous rodent Tslp locus, an endogenous rodent Tslpr locus, or an endogenous rodent Il7ra locus), so as to form a humanized gene as described herein above. Typically, the 5′ and 3′ flanking rodent sequences are the nucleotide sequences that flank the corresponding rodent nucleotide sequence at the target rodent locus that is to be replaced by the human nucleotide sequence. In some embodiments, a targeting vector comprises a humanized gene as described herein above. In some embodiments, a targeting vector comprises a humanized Tslp gene comprising a human TSLP nucleotide sequence and a rodent Tslp nucleotide sequence, as described herein above. In some embodiments, a targeting vector comprises a humanized Tslpr gene comprising a human TSLPR nucleotide sequence and a rodent Tslpa nucleotide sequence, as described herein above. In some embodiments, a targeting vector comprises a humanized Il7ra gene comprising a human IL7RA nucleotide sequence and a rodent Il7ra nucleotide sequence, as described herein above.

In some embodiments, a targeting vector comprises a selection marker gene. The selection marker gene can be inserted in an intron of the human genomic sequence to be integrated. In some embodiments, a selection marker gene is provided as a self-deleting cassette which can be deleted after a successful integration of the human nucleotide sequence.

In an exemplary embodiment, a targeting vector is generated from a bacterial artificial chromosome (BAC) clone carrying a rodent Tslp, Tslpr, or 117ra genomic DNA using bacterial homologous recombination and VELOCIGENE® technology (see, e.g., U.S. Pat. No. 6,586,251 and Valenzuela et al. (2003) Nature Biotech. 21(6):652-659, incorporated herein by reference in their entireties). As a result of bacterial homologous recombination, a rodent genomic sequence is deleted from the BAC clone, and a human nucleotide sequence is inserted, resulting in a modified BAC clone carrying the human nucleotide sequence, flanked with 5′ and 3′ rodent homology arms. In some embodiments, the human nucleotide sequence can be a cDNA sequence or a human genomic DNA. The modified BAC clone, once linearized, can be introduced into rodent embryonic stem (ES) cells.

In some embodiments, the present invention provides use of a targeting vector as described herein to make a modified rodent embryonic stem (ES) cell. A targeting vector can be introduced into a rodent ES cell by, e.g., electroporation. Both mouse ES cells and rat ES cells have been described in the art. See, e.g., U.S. Pat. Nos. 7,576,259, 7,659,442, 7,294,754, and US 2008-0078000 A1 (all of which are incorporated herein by reference in their entireties) that describe mouse ES cells and the VELOCIMOUSE® method for making a genetically modified mouse; US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties) that describe rat ES cells and methods for making a genetically modified rat, which can be used to make a modified rodent embryo, which in turn can be used to make a rodent animal.

In some embodiments, ES cells having a desirable human nucleotide sequence (e.g., human TSLP, human TSLPR, or human IL7RA nucleotide sequence) integrated in the genome can be selected. In some embodiments, ES cells are selected based on loss of rodent allele and/or gain of human allele assays. In some embodiments, selected ES cells are then used as donor ES cells for injection into a pre-morula stage embryo (e.g., 8-cell stage embryo) by using the VELOCIMOUSE® method (see, e.g., U.S. Pat. Nos. 7,576,259, 7,659,442, 7,294,754, and US 2008-0078000 A1, all of which are incorporated by reference in their entireties), or methods described in US 2014/0235933 A1 and US 2014/0310828 A1, which are both incorporated by reference in their entireties. In some embodiments, an embryo comprising the donor ES cells is incubated and implanted into a surrogate mother to produce an F0 rodent. Rodent pups bearing a human nucleotide sequence can be identified by genotyping of DNA isolated from tail snips using loss of rodent allele and/or gain of human allele assays.

In some embodiments, rodents heterozygous for a humanized gene can be crossed to generate homozygous rodents.

A humanized rodent as described herein (i.e., a rodent comprising a humanized Tslp gene, a humanized Tslpr gene, a humanized Il7ra gene, or a combination thereof) can be bred or crossed with another rodent. Accordingly, methods of breeding as well as progenies obtained from such breeding are also embodiments of this disclosure.

In some embodiments, a method is provided which comprises breeding a first rodent as described hereinabove, e.g., a rodent whose genome comprises a humanized Tslp gene, a humanized Tslpr gene, a humanized Il7ra gene, or a combination thereof, with a second rodent, resulting in a progeny rodent whose genome comprises the humanized Tslp, Tslpr, and/or Il7ra gene(s). The progeny may possess other desirable phenotypes or genetic modifications inherited from the second rodent used in the breeding. In some embodiments, the progeny rodent is heterozygous for the humanized gene or genes from the first rodent. In some embodiments, the progeny rodent is homozygous for the humanized gene(s) from the first rodent. In some embodiments, the second rodent used in breeding comprises one or more of an additional genetic modifications such as a humanized Sirpα gene, RAG2−/− and IL-2RG−/−, a humanized Tpo gene, or a humanized GM-CSF/IL-3 locus.

In some embodiments, a progeny rodent is provided whose genome comprises a humanized Tslp gene, a humanized Tslpr gene, a humanized Il7ra gene, or a combination thereof, wherein the progeny rodent is produced by a method comprising breeding a first rodent whose genome comprises the humanized gene or genes, with a second rodent. In some embodiments, the progeny rodent is heterozygous for the humanized gene or genes from the first rodent. In some embodiments, the progeny rodent is homozygous for the humanized gene or genes from the first rodent.

In some embodiments, disclosed herein is an in vitro method for generating a genetically modified rodent cell, comprising introducing into a rodent cell a targeting vector comprising a human TSLP nucleic sequence that encodes at least a substantial portion of the mature protein sequence of a human TSLP protein, flanked by rodent homology arms that mediate integration of the human TSLP nucleotide sequence into an endogenous rodent Tslp locus, which results in replacement of a rodent Tslp genomic DNA with the human TSLP nucleic acid sequence to form a humanized Tslp gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

In some embodiments, disclosed herein is an in vitro method for generating a genetically modified rodent cell, comprising introducing into a rodent cell a targeting vector comprising a human TSLPR nucleic sequence that encodes at least a substantial portion of the ectodomain of a human TSLPR protein, flanked by rodent homology arms that mediate integration of the human TSLPR nucleotide sequence into an endogenous rodent Tslpr locus, which results in replacement of a rodent Tslpr genomic DNA with the human TSLPR nucleic acid sequence to form a humanized Tslpr gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

In some embodiments, disclosed herein is an in vitro method for generating a genetically modified rodent cell, comprising introducing into a rodent cell a targeting vector comprising a human IL7RA nucleic sequence that encodes at least a substantial portion of the ectodomain of a human IL7RA protein, flanked by rodent homology arms that mediate integration of the human IL7RA nucleotide sequence into an endogenous rodent Il7ra locus, which results in replacement of a rodent Il7ra genomic DNA with the human IL7RA nucleic acid sequence to form a humanized Il7ra gene as described herein, thereby generating a genetically modified rodent cell. In some embodiments, the rodent cell is mouse cell or a rat cell. In some embodiments, the rodent cell is a rodent ES cell, and the method generates a genetically modified rodent ES cell.

### Methods Employing the Humanized Rodents

Rodents disclosed herein provide a useful in vivo system and source of biological materials for identifying and testing compounds for their potential to treat human diseases, including particularly diseases associated with TSLP signalling, such as Th2-driven allergic diseases, asthma, and cancer.

In some embodiments, rodent animals disclosed herein are used to develop agents that target TSLP signaling through, e.g., targeting human TSLP, human TSLPR, or human IL7RA. In some embodiments, rodents disclosed herein are used to screen and develop candidate agents (e.g., antibodies) that specifically bind to human TSLP, human TSLPR, or human IL7RA. In some embodiments, rodent animals disclosed herein are used to determine the binding profile of an agent (e.g., an antibody). In some embodiments, rodent animals disclosed herein are used to measure the effect of blocking or modulating human TSLP, TSLPR, or IL7RA activity. In some embodiments, a rodent animal disclosed herein is exposed to a candidate agent that binds to and inhibits human TSLP, and is analyzed for effects on human TSLP-dependent processes.

In some embodiments, a genentically modified rodent described herein is used as a model of allergic diseases. In some embodiments, the allergic disease involves airway inflammation (e.g., asthma).

In some embodiments, the ova-alum model of lung inflammation is used to assess the Tslp signaling. The ova-alum model of lung inflammation has been well documented in the art (Al-Shami et al., JEM Vol. 202, No. 6, 829-839, 2005; Chu et al., J. Allergy Clin Immunol 2013; 131:187-200, incorporated herein by reference in their entireties). In some embodiments, OVA emulsified in aluminum hydroxide, or aluminum hydroxide alone (as control) is administered intraperitoneally to rodent animals (e.g., mice such as mice humanized for one or more of Tslp, Tslpr, and/or Il7ra as disclosed herein, or wild type mice without humanization). Mice are then challenged intranasally with OVA, and are subsequently analyzed for parameters indicate of lung inflammation, including, e.g., serum ova-specific IgE and ova-specific-IgG1, goblet cell metaplasia, and/or lung tissue eosinophilia. In some embodiments, lung expression of Muc5ac, a representative mucin gene overexpressed in airways of asthmatic lungs, is analyzed after the challenge and may serve as a surrogate endpoint for goblet cell metaplasia. In an exemplary protocol, 50 μg of OVA emulsified in 2 mg of aluminum hydroxide or 2 mg of aluminum hydroxide alone is administered intraperitoneally to rodent animals (e.g., mice such as mice doubly humanized for Tslp and Tslpr, mice triply humanized for Tslp, Tslpr, and Il7ra, as disclosed herein, or wild type mice without humanization, on days 1 and 14). Anesthetized mice are challenged intranasally with 150 μg of OVA in PBS for 4 days, starting on day 21. Mice are analyzed 24 hours after the last challenge for serum Ova-specific IgE and Ova-specific-IgG1, goblet cell metaplasia, and/or lung tissue eosinophilia. In some embodiments, lung expression of Muc5ac, a representative mucin gene overexpressed in airways of asthmatic lungs, is analyzed after the challenge and may serve as a surrogate endpoint for goblet cell metaplasia.

In some embodiments, airway inflammation can be induced in a rodent by intranasal administration of an allergen (e.g., house dust mite extract or “HDM” model) in one or more doses for a period of time, and airway inflammation can be measured based on mucus accumulation, eosinophilic infiltrating cells in bronchoalveolar lavage fluid, levels of total circulating IgE, and/or alteration in expression profile measurable by microarray expression analysis. The effect of a candidate therapeutic agent can be determined by measuring whether the extent of airway inflammation, either in the ova-alum model or the HDM model, is reduced as a result of the administration of the agent. The allergen used for inducing airway inflammation and the agent being tested can be administered simultaneously or at different times. In some embodiments, the allergen is given to the rodent in one or more doses, and the agent being tested is administered to the rodent after at least one dose of the allergen has been given to the rodent.

In some embodiments, the allergic disease involves skin inflammation or atopic dermatitis. Skin inflammation can be induced in a rodent by creating skin injury and exposing the injured skin to an allergen (e.g., bacterial toxin or house dust mite extract) in one or more doses for a period of time. The effect of an agent can be determined by measuring whether skin inflammation (as determined by assessing, e.g., IgE levels, pruritis, thickening of the epidermis, and other typical symptoms of atopic dermatitis) is reduced as a result of administration of the agent.

In some embodiments, rodent animals disclosed herein are used as a animal model for cancer such as a Th2-driven cancer, in order to, e.g., assess the efficacy of a therapeutic drug targeting human cancer cells. In various embodiments, a rodent animal disclosed herein is engrafted with human cancer cells, and a drug candidate targeting such human cancer cells is administered to the rodent animal. The therapeutic efficacy of the drug is then determined by monitoring the human cancer cells in the rodent animal after the administration of the drug, e.g., by assessing whether growth or metastasis of the human cancer cells in the rodent animal is inhibited as a result of the administration of the drug. Human cancer cells suitable for engraftment into a rodent animal include, e.g., breast cancer cells, lung cancer cells, pancreatic cancer cells, colon cancer cells, melanoma, among others. Drugs that can be tested in the non-human animals include both small molecule compounds, i.e., compounds of molecular weights of less than 1500 kD, 1200 kD, 1000 kD, or 800 dalton, and large molecular compounds (such as proteins, e.g., antibodies), which have intended therapeutic effects for the treatment of human diseases and conditions by targeting (e.g., binding to and/or acting on) human cells.

The present description is further illustrated by the following examples, which should not be construed as limiting in any way. The contents of all cited references (including literature references, issued patents, and published patent applications as cited throughout this application) are hereby expressly incorporated by reference in their entireties.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Example 1. Generation of Humanized Tslp Mice

The mouse Tslp locus was humanized by using VELOCIGENE® technology (see, e.g., U.S. Pat. No. 6,586,251 and Valenzuela et al. (2003) High-throughput engineering of the mouse genome couple with high-resolution expression analysis, Nat. Biotech. 21(6): 652-659, both incorporated herein by reference in their entireties). The resulting humanized Tslp locus included a mouse Tslp promoter, mouse Tslp exon 1, mouse Tslp exon 2 in part (from the 5′ end of exon 2 through the codon encoding the last amino acid of the mouse Tslp signal peptide), human TSLP exon 1 in part (from the codon encoding the first amino acid of the mature human TSLP protein to the 3′ end of exon 1) through the STOP codon in human TSLP exon 4, followed by a mouse Tslp 3′ UTR and downstream mouse genomic sequences. See FIGS. 1A-1C.

To humanize a mouse Tslp locus, a targeting nucleic acid construct was generated based on the following mouse and human sequences:

TABLE 4

| | NCBI | RefSeq | UniProt | Genomic | |
|---|---|---|---|---|---|
| | GeneID | mRNA ID | ID | Assembly | Location |
| Mouse | 53603 | NM_021367 | Q9JIE6 | GRCm38/mm10 | chr18: 32,815,383-32,819,799 (+) |
| Tslp | | | | | |
| Human | 85480 | NM_033035 | Q969D9 | GRCh38/hg38 | chr5: 111,070,062-111,078,026 (+) |
| TSLP | | | | | |

TABLE 5

| | Genome | | | Length |
|---|---|---|---|---|
| | Build | Start | End | (bp) |
| 5′ mouse | GRCm38/ | Chr18: 32701299 | Chr18: 32815620 | 114322 |
| Arm | mm10 | | | |
| Human | GRCh38/ | Chr5: 111071975 | Chr5: 111,073,902 | 1928 |
| Genomic | hg38 | | | |
| Fragment 1 | | | | |
| Human | GRCh38/ | chr5: 111,073,903 | Chr5: 111,076,074 | 2172 |
| Genomic | hg38 | | | |
| Fragment 2 | | | | |
| 3′ mouse | GRCm38/ | Chr18: 32819107 | Chr18: 32884365 | 65259 |
| Arm | mm10 | | | |

The targeting nucleic acid construct contained from 5′ to 3′:

(i) a 5′ mouse homology arm of 114.3 kb upstream of the codon in mouse Tslp exon 2 that encodes the first amino acid of the mature mouse Tslp protein; see FIG. 1B;

(ii) a human TSLP genomic sequence of 1.9 kb designated as “Human Genomic Fragment 1”, which begins from the codon in human TSLP exon 1 that encodes the first amino acid of the mature human TSLP protein (amino acid 29), and ends at 257 bp after the end of human TSLP exon 3; see FIG. 1B;

(iii) a selection cassette of 4.4 kb designated as “Floxed HUb-Puro” (a puromycin resistance gene operably linked to a human ubiquitin promoter, flanked by LoxP sites), inserted in intron 3 of human TSLP; see FIG. 1B;

(iv) a human TSLP genomic sequence of 2.2 kb designated as “Human Genomic Fragment 2”, which begins from 258 bp after the end of human TSLP exon 3 and ends at the STOP codon in human TSLP exon 4; see FIG. 1B; and

(v) a 3′ mouse homology arm of about 65.3 kb, which includes the 3′ UTR sequence of mouse Tslp exon 5 and the downstream mouse genomic sequence; see FIG. 1B.

The targeting nucleic acid construct was electroporated into F1H4 mouse embryonic stem (ES) cells. Successful integration was confirmed by a modification of allele (MOA) assay as described, e.g., in Valenzuela et al., supra. Primers and probes used for the MOA assay for detecting the presence of human TSLP sequences and confirms the loss and/or retention of mouse Tslp sequences are described in Table 6, and their locations are shown in FIG. 1B.

TABLE 6
7466hTU

| | |
|---|---|
| Fwd | CAGATGCGGACATCCAAAGGAT |
| | (SEQ ID NO: 9) |
| Probe (FAM) | TACTCACAAGCATAGTGCTATGTGCA |
| | (SEQ ID NO: 10) |
| Rev | CCCTTCCCTCAAGCCATAAC |
| | (SEQ ID NO: 11) |

7466hTD

| | |
|---|---|
| Fwd | GCCCAGTGTACTACTCAAAGGTA |
| | (SEQ ID NO: 12) |
| Probe (Cal) | TACTGCAATCCTCTTTAAAATAAGC |
| | (SEQ ID NO: 13) |
| Rev | CCCATTGTCTAGATGTGTCACAGA |
| | (SEQ ID NO: 14) |

7466mTU

| | |
|---|---|
| Fwd | GGCTGACAACAGATATGGATATTGG |
| | (SEQ ID NO: 15) |
| Probe (FAM) | ACTGCTTGGTACAGAATGGGAATCC |
| | (SEQ ID NO: 16) |
| Rev | CACGGCTTCATGTCTTAGCTG |
| | (SEQ ID NO: 17) |

7466mTD

| | |
|---|---|
| Fwd | GTGCTGAGAGACAGGGCATTC |
| | (SEQ ID NO: 18) |
| Probe (Cal) | TGGAGAAGCACATGCAATCATACCGT |
| | (SEQ ID NO: 19) |
| Rev | GGCTGAGTGGCACTATGTTTC |
| | (SEQ ID NO: 20) |

After a correctly targeted ES cell clone was selected, the puromycin selection cassette was excised. The coding sequence of the humanized Tslp gene and the encoded amino acid sequence are set forth in SEQ ID NO: 6 and SEQ ID NO: 5, respectively. An alignment of mouse Tslp (SEQ ID NO: 1), human TSLP (SEQ ID NO: 3), and humanized Tslp (SEQ ID NO: 5) protein sequences is provided in FIG. 1F.

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference in their entireties). The mouse embryo comprising the donor ES cells was incubated in vitro and then implanted into a surrogate mother to produce an F0 mouse fully derived from the donor ES cells. Mice bearing a humanized Tslp gene were identified by genotyping using the MOA assay described above. Mice heterozygous for the humanized Tslp gene were bred to homozygousity.

To determine whether mice homozygous for the Tslp humanization expressed the humanized Tslp protein, mice were euthanized and bled via cardiac puncture. Blood was collected into serum separator tubes and serum prepared. Human TSLP levels in the serum was determined using Human Quantikine TSLP ELISA (R&D systems; Cat #DTSLPO) according to manufacturer's instructions. Recombinant murine Tslp (R&D systems Cat #555-TS-010) at 1000 pg/mL was also used as a negative control to validate species specificity of the ELISA (data not shown) and normal human serum (NETS) was used as positive control. Mice heterozygous for the Tslp humanization as described above were found to express mature human TSLP in serum (FIG. 1G).

### Example 2. Generation of Humanized Tslpr Mice

The mouse Tslpr locus was humanized by using VELOCIGENE® technology (see, e.g., U.S. Pat. No. 6,586,251 and Valenzuela et al. (2003) High-throughput engineering of the mouse genome couple with high-resolution expression analysis, Nat. Biotech. 21(6): 652-659, both incorporated herein by reference in their entireties). The resulting humanized Tslpr locus included a mouse Tslpr promoter, mouse Tslpr exon 1 (including the 5′ UTR and sequence encoding the mouse Tslpr signal peptide and the first 7 amino acids of the mouse Tslpr mature protein), mouse Tslpr intron 1 in part (up to 328 bp before exon 2), human TSLPR intron 1 in part (beginning at 909 bp before exon 2), human TSLPR exon 2 through the first 47 bp of exon 6 (encoding substantially the human TSLPR ectodomain, i.e., from amino acid 27 to just before the transmembrane domain), mouse Tslpr exon 6 beginning from the 48th bp through exon 8 (encoding the mouse Tslpr transmembrane and intracellular domains, and including the mouse Tslpr 3′ UTR), followed by downstream mouse genomic sequences. See FIGS. 2A-2C.

To humanize the mouse Tslpr locus, a targeting nucleic acid construct was generated based on the following sequence information:

TABLE 7

| | NCBI | RefSeq | UniProt | Genomic | |
|---|---|---|---|---|---|
| | Gene ID | mRNA ID | ID | Assembly | Location |
| Mouse | 57914 | NM_001164735 | Q8CII9 | GRCm38/mm10 | chr5: 109,554,709-109,558,993 (−) |
| Tslpr | | | | | |
| Human | 64109 | NM_022148 | Q9HC73 | GRCh38/hg38 | Chr X: 1,187,549-1,212,750 (−) |
| TSLPR | | | | | |

TABLE 8

| | Genome | | | Length |
|---|---|---|---|---|
| | Build | Start | End | (bp) |
| 5′ mouse | GRCm38/ | Chr5: 109557943 | Chr5: 109586999 | 29057 |
| Arm | mm10 | | | |
| Human | GRCh37/ | ChrX: 1314968 | ChrX: 1328710 | 13743 |
| Insert | hg37 | | | |
| 3′ mouse | GRCm38/ | Chr5: 109422337 | Chr5: 109555580 | 133244 |
| Arm | mm10 | | | |

The targeting nucleic acid construct contained from 5′ to 3′:

(i) a 5′ mouse homology arm of about 29.1 kb up to 328 bp before mouse Tslpr exon 2; see FIG. 2B;

(ii) a self deleting selection cassette containing a neomycine resistance gene operably linked to a human ubiquitin promoter, flanked by LoxP sites (“Floxed HUb-Neo”), see FIG. 2B;

(iii) a human TSLPR nucleic acid sequence of 13743 bp, which begins from 909 bp before exon 2 through the first 47 bp in exon 6 of human TSLPR, which encodes substantially the human TSLPR ectodomain, i.e., from amino acid 27 to amino acid 231 (in human TSLPR: amino acids 1-22 constituting the signal peptide and amino acids 232 to 252 constituting the transmembrane domain), see FIG. 2B; and

(iv) a 3′ mouse homology arm of about 133.2 kb, which begins from 48th bp in exon 6 through exon 8, encodes the mouse Tslpr transmembrane domain and intracellular domain, and includes the mouse Tslpr 3′ UTR followed by downstream mouse genomic sequences; see FIG. 2B.

The targeting nucleic acid construct was electroporated into mouse embryonic stem (ES) cells. Successful integration was confirmed by a modification of allele (MOA) assay as described, e.g., in Valenzuela et al., supra. Primers and probes used for the MOA assay for detecting the presence of human TSLPR sequences and confirms the loss and/or retention of mouse Tslpr sequences are set forth in Table 9, and their locations are indicated in FIG. 2B. After a correctly targeted ES cell clone was selected, the neomycin selection cassette can be excised. The genomic sequences of the targeted (humanized) Tslpr allele with and without the cassette are set forth in SEQ ID NO: 63 and 64, respectively. The coding sequence of the humanized Tslpr gene and the encoded amino acid sequence are set forth in SEQ ID NO: 26 and SEQ ID NO: 25, respectively. An alignment of mouse Tslpr (SEQ ID NO: 21), human TSLPR (SEQ ID NO: 23), and humanized Tslpr (SEQ ID NO: 25) protein sequences is provided in FIG. 2F.

TABLE 9
7558hTU

| | |
|---|---|
| Fwd | TGCCTCACCGTGAACTTCATG |
| | (SEQ ID NO: 29) |
| Probe (FAM) | CGTCTCTCTGTGTCTAGCAGAAGGA |
| | (SEQ ID NO: 30) |
| Rev | TCACCTGCACGGTTTCTAAATTG |
| | (SEQ ID NO: 31) |

7558hTD

| | |
|---|---|
| Fwd | CAGCCGCACGTCATGTTG |
| | (SEQ ID NO: 32) |
| Probe (Cal) | TGACAGCCGCCTTTTCATTTTGTTTCA |
| | (SEQ ID NO: 33) |
| Rev | GGACAGCTTTGGTTTGGGA |
| | (SEQ ID NO: 34) |

7558mTU

| | |
|---|---|
| Fwd | GCTAGCTGCTCATTTGCATATTCG |
| | (SEQ ID NO: 35) |
| Probe (FAM) | AGAAGCGCTTTCCATATTCATGAGCCC |
| | (SEQ ID NO: 36) |
| Rev | GGGCGACACCTCATTTGCAT |
| | (SEQ ID NO: 37) |

7558mTD

| | |
|---|---|
| Fwd | GGGTCTGGGTAAGATGAACTCA |
| | (SEQ ID NO: 38) |
| Probe (Cal) | TCGGCTCCTGGATGCTTGACA |
| | (SEQ ID NO: 39) |
| Rev | CATCCGGGTCACCAATGATG |
| | (SEQ ID NO: 40) |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein in their entireties by reference). The mouse embryo comprising the donor ES cells was incubated in vitro and then implanted into a surrogate mother to produce an F0 mouse fully derived from the donor ES cells. Mice bearing a humanized Tslpr gene were identified by genotyping using the MOA assay described above. Mice heterozygous for the humanized Tslpr gene were bred to homozygousity.

### Example 3. Generation of Humanized Il7ra Mice

The mouse Il7ra locus was humanized by using VELOCIGENE® technology (see, e.g., U.S. Pat. No. 6,586,251 and Valenzuela et al. (2003) High-throughput engineering of the mouse genome couple with high-resolution expression analysis, Nat. Biotech. 21(6): 652-659, both incorporated herein by reference in their entireties). The resulting humanized Il7ra locus included a mouse Il7ra promoter, mouse Il7ra exon 1 in part (including the 5′ UTR and the first 68 bps beginning from the start codon, and encoding the mouse Il7ra signal peptide and the first 3 amino acids of the mouse Il7ra mature protein), human IL7RA exon 1 in part (the last 14 bps in exon 1), human IL7RA intron 1, human IL7RA exon 2 through exon 5, human IL7RA intron 5 in part, mouse Il7ra intron 5 in part, mouse Il7ra exon 6 through exon 8 (encoding the last two amino acids of the mouse Il7ra ectodomain, the transmembrane domain and intracellular domain of mouse Il7ra, and including the mouse Il7ra 3′ UTR). See FIGS. 3A-3F.

To humanize the mouse Il7ra locus, a targeting nucleic acid construct was generated based on the following sequence information:

TABLE 10

| | NCBI | RefSeq | UniProt | Genomic | |
|---|---|---|---|---|---|
| | Gene ID | mRNA ID | ID | Assembly | Location |
| Mouse | 16197 | NM_008372 | P16872 | GRCm38/mm10 | chr15: 9,505,788-9,530,176 (−) |
| Il7ra | | | | | |
| Human | 3575 | NM_002185 | P16871 | GRCh38/hg38 | chr5: 35,852,695-35,879,603 (+) |
| IL7RA | | | | | |

TABLE 11

| | Genome | | | Length |
|---|---|---|---|---|
| | Build | Start | End | (bp) |
| 5′ mouse | GRCm38/ | Chr15: 9529675 | Chr15: 9578484 | 48810 |
| Arm | mm10 | | | |
| Human | GRCh38/ | Chr5: 35857046 | Chr5: 35874277 | 17232 |
| Insert | hg38 | | | |
| 3′ mouse | GRCm38/ | Chr15: 9386119 | Chr15: 9510439 | 124321 |
| Arm | mm10 | | | |

The targeting nucleic acid construct contained from 5′ to 3′:

(i) a 5′ mouse homology arm of about 48.8 kb up (mouse Il7ra 5′ sequence through exon 1 in part, i.e., the 5′ UTR and the first 68 bps beginning from the start codon); see FIG. 3B and FIG. 3E;

(ii) a Human Genomic Fragment 1 of 126 bp (SEQ ID NO: 69) (which includes the last 14 bps of exon 1 and the first 112 bps of intron 1 of human IL7RA); see FIG. 3B and FIG. 3E;

(iii) a self deleting selection cassette of about 5.2 kb containing a hygromycin resistance gene operably linked to a human ubiquitin promoter, flanked by LoxP sites (“Floxed HUb-Hyg”); see FIG. 3B;

(iv) a Human Genomic Fragment 2 of 17106 bp, which includes a 3′ portion of intron 1, exon 2 through exon 5, and a 5′ portion of intron 5 of human IL7RA; see FIG. 3B; and

(v) a 3′ mouse homology of about 124.3 kb, which includes a 3′ portion of intron 5, exon 6 through exon 8 of mouse Il7ra (including the mouse Il7ra 3′ UTR), followed by downstream mouse genomic sequences; see FIGS. 3B and 3F. Exon 6 through exon 8 of mouse Il7ra encodes the last two amino acids (Gly-Trp) of the ectodomain, the transmembrane domain and intracellular domain of mouse Il7ra.

The targeting nucleic acid construct was electroporated into F1H4 mouse embryonic stem (ES) cells. Successful integration was confirmed by a modification of allele (MOA) assay as described, e.g., in Valenzuela et al., supra. Primers and probes used for the MOA assay for detecting the presence of human IL7RA sequences and confirms the loss and/or retention of mouse Il7ra sequences are set forth in Table 12, and their locations are indicated in FIG. 3B. After a correctly targeted ES cell clone was selected, the hygromycin selection cassette can be excised. The genomic sequences of the targeted (humanized) Il7ra allele with and without the cassette are set forth in SEQ ID NO: 65 and 66, respectively. The coding sequence of the humanized Il7ra gene and the encoded amino acid sequence are set forth in SEQ ID NO: 46 and SEQ ID NO: 45, respectively. An alignment of mouse Il7ra (SEQ ID NO: 41) and human IL7RA (SEQ ID NO: 43) protein sequences is provided in FIG. 3F.

TABLE 12
7266hTU2

| | |
|---|---|
| Fwd | GGGATCAATACTATGGGTGGTTTATAA |
| | (SEQ ID NO: 49) |
| Probe (FAM) | ACCTCAGTATTCTCAAGAAG |
| | (SEQ ID NO: 50) |
| Rev | CTACACTTGGGAGTGAAATGCATT |
| | (SEQ ID NO: 51) |

7266hTD

| | |
|---|---|
| Fwd | GGAGGGCACTCTTACACTTTC |
| | (SEQ ID NO: 52) |
| Probe (Cal) | TTGGAGAATGACTTGCCTGCTGTC |
| | (SEQ ID NO: 53) |
| Rev | CCTCTGCTTCCTTGTTCTTCACA |
| | (SEQ ID NO: 54) |

7266mTU

| | |
|---|---|
| Fwd | CAGGGCAAGCAAGAATTTAGCA |
| | (SEQ ID NO: 55) |
| Probe (FAM) | TGTGGGTATTAATCACCAGGACAGAGGG |
| | (SEQ ID NO: 56) |
| Rev | ACAAGCCATTTGCAGTATTGTCA |
| | (SEQ ID NO: 57) |

7266mTD2

| | |
|---|---|
| Fwd | TGGGTCAGTTTGGCTATCCAT |
| | (SEQ ID NO: 58) |
| Probe (Cal) | TCTTTTCCCAGAACAATGAAGATGCTATGG |
| | (SEQ ID NO: 59) |
| Rev | TGCTTTGGGTACTGTCCTGAAG |
| | (SEQ ID NO: 60) |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein in their entireties by reference). The mouse embryo comprising the donor ES cells was incubated in vitro and then implanted into a surrogate mother to produce an F0 mouse fully derived from the donor ES cells. Mice bearing a humanized Il7ra gene were identified by genotyping using the MOA assay described above. Mice heterozygous for the humanized Il7ra gene were bred to homozygosity.

### Example 4. Ova-Alum Induced Type 2 Driven Inflammation in Wild Type, Double Humanized Mice (Tslp^{hu/hu}/Tslpr^{hu/hu}) and Triple Humanized Mice (Tslp^{hu/hu}/Tslpr^{hu/hu}/Il7Ra^{hu/hu})

To confirm that the various humanized mice strains have comparable pathology in a model of type 2 driven inflammation, the ovalbumin (OVA)/alum-induced lung inflammation model was employed, wherein a role for TSLP has been previously reported (Chu, et al., J Allergy Clin Immunol 2013; 131:187-200.e1-8, the entire contents of which are incorporated herein by reference). Traditional type 2 inflammation endpoints such as circulating levels of antigen specific IgE and IgG1, lung tissue eosinophil infiltration and lung expression of Muc5ac, a representative mucin gene overexpressed in airways of asthmatic lungs, and a surrogate endpoint for goblet cell metaplasia (GCM) (Wills-Karp, et al., Science 1998; 282:2258-61, the entire contents of which are incorporated herein by reference) were assessed in three strains of mice: wild type mice (WT), double humanized mice (Tslp^{hu/hu}/Tslpr^{hu/hu}) and triple humanized mice (Tslp^{hu/hu}/Tslpr^{hu/hu}/IL7R^{hu/hu}). The humanization for each of the Tslp, Tslpr and IL7R molecules is as described in Example 1, 2 and 3, respectively.

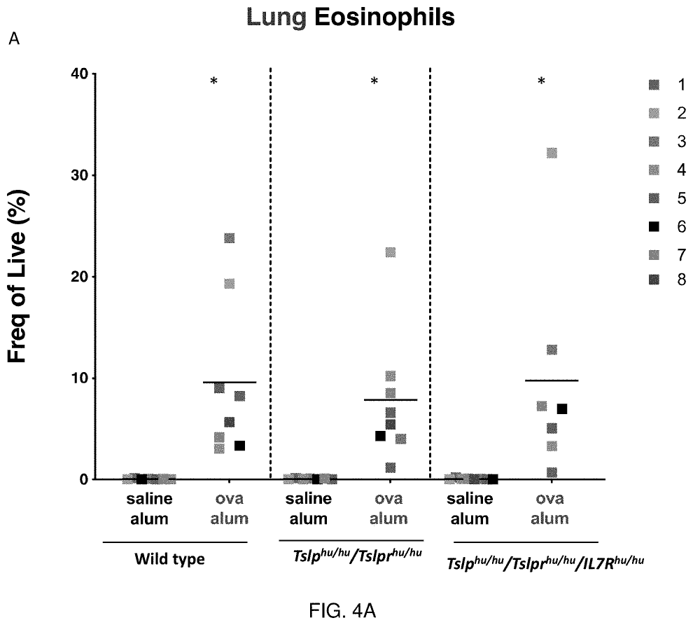

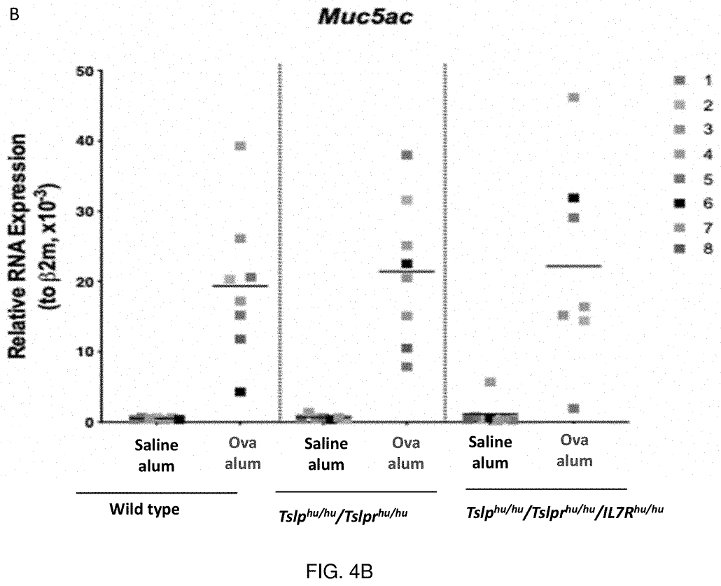

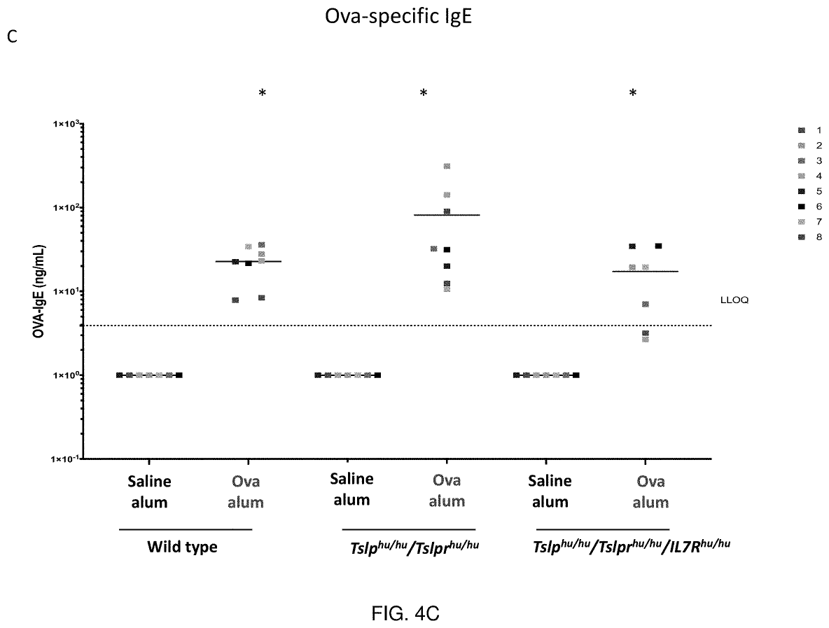

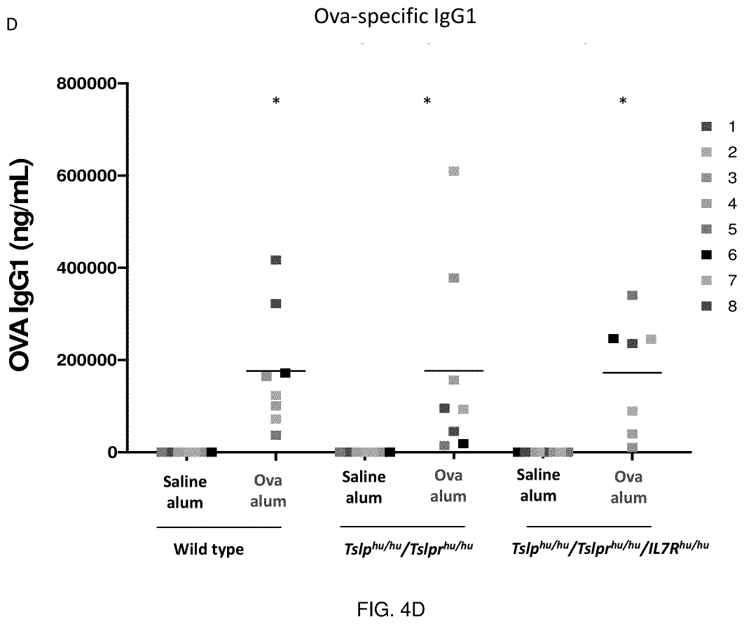

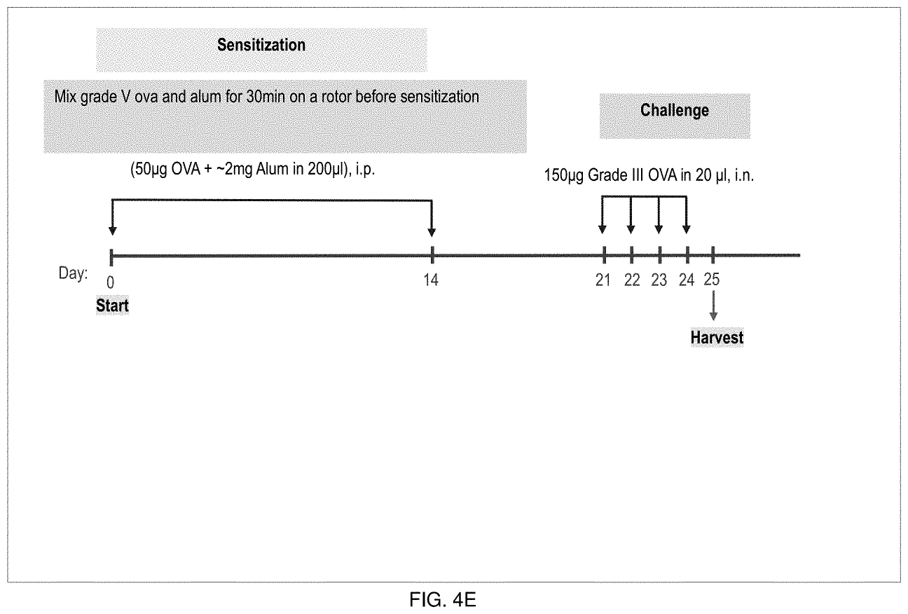

As the lung is a highly vascularized organ, cells infiltrating the lung (lung tissue) from those circulating in the lung vasculature (lung circulating) were distinguished using a CD45-based intravascular labeling technique (Anderson, et al., Nat Protoc 2014; 9:209-22, the entire contents of which are incorporated herein by reference) prior to gating on eosinophils. As expected, TSLP/OVA administration induced an increase in lung tissue eosinophils at comparable levels across mouse strains (FIG. 4A). In addition, TSLP/OVA induced comparable levels of lung expression of Muc5ac (FIG. 4B) as well as comparable levels of circulating levels of Ova specific IgE (FIG. 4C) and Ova specific IgG1 (FIG. 4D).

### Methods

Ova-alum-induced lung inflammation. 50 μg of OVA (grade V; Sigma Aldrich) emulsified in 2 mg of aluminum hydroxide (Sigma Aldrich) or 2 mg of aluminum hydroxide alone was administered intraperitoneally to WT Balb/c mice on days 1 and 14. Anesthetized mice were challenged intranasally with 150 μg of OVA (grade III; Sigma Aldrich) in 20 μL of PBS for 4 days, starting on day 21. Mice were analyzed 24 hours after the last challenge.

At the end of the study, mice were sacrificed and blood and lung collected for analysis of eosinophil infiltrates in lung tissue, lung gene expression by real-time qPCR and circulating serum immunoglobulin levels.

Flow cytometric analysis. To enable flow cytometric analysis of circulating versus tissue-infiltrating immune cells in the lung, mice were injected intravenously with an anti-CD45 BV650 antibody (BD Biosciences) 5 minutes prior to sacrifice to selectively label immune cells still in the vasculature while leaving cells that had infiltrated the lung parenchyma unlabeled. Mouse caudal lung lobes were digested to prepare a single-cell suspension using a solution of Liberase TH (Roche) and DNase I (Roche), followed by mechanical dissociation. Cells were then stained with LIVE/DEAD Fixable Dead Cell Stain (BD Biosciences) to allow exclusion of dead cells followed by antibodies against: CD45, CD26, Siglec-F, Ly6G, Ly6C, CD11b, CD19, SIRPα, CD23, CD127, Sca-1, CD44, CD4, CD8, TCRb, CD69 CD62L (BD Biosciences); CD64, XCR1, I-A/I-E, CD11c, CD301b (Biolegend); MerTK, ST2 (eBioscience). Samples were acquired on an LSR Fortessa X-20 or a FACSymphony cell analyzer using the HTS attachment (BD). Data analysis was performed using FlowJo v10 Software (BD).

Measurement of serum IgE and antigen-specific IgE or IgG1. Whole blood was collected into Microtainer SST serum tubes and pelleted by centrifuging at 15,000 g for 10 min at 4° C. Serum samples were used to determine concentrations: total IgE concentrations by IgE sandwich ELISA OptEIA kit (BD Biosciences); total anti-Ova IgE and anti-Ova IgG1 concentrations by indirect ELISA (Chondrex); total anti-HDM IgE by sandwich ELISA (Chondrex); and total anti-HDM IgG1 titers by in-house sandwich ELISA.

Manufacturer's instructions were followed for all ELISA kits.

Measurement of Muc5ac. Lung Muc5ac gene expression was detected in harvested lung tissue by real time qPCR and normalized to a housekeeping gene. Briefly, at the end of the study, mice were exsanguination and the accessory lobe of the right lung from each mouse was removed, placed into tubes containing 400 μL of RNA Later and stored at −20° C. All samples were homogenized in TRIzol and chloroform was used for phase separation. The aqueous phase, containing total RNA, was purified using MagMAX™-96 for Microarrays Total RNA Isolation Kit (Ambion by Life Technologies) according to manufacturer's specifications. Genomic DNA was removed using RNase-Free DNase Set (Qiagen). mRNA was reverse-transcribed into cDNA using SuperScript® VILO™ Master Mix (Invitrogen by Life Technologies). cDNA was diluted to 2 ng/uL and 10 ng cDNA was amplified with the SensiFAST Probe Hi-ROX (Meridian) using the ABI 7900HT Sequence Detection System (Applied Biosystems). An internal control housekeeping gene was used to normalize any cDNA input differences. Fold change relative to control mice of lung tissue mRNA expression levels were measured and expressed relative to housekeeping mRNA expression.

TABLE 13

| | | | | | # of mice |
|---|---|---|---|---|---|
| Group | MAID | Genotype | Sensitization i.p. | Challenge, i.n. | (initial/harvest) |
| A | 50500 | Wild Type | saline + alum | saline | 7/7 |
| | | (C57BL/6NTac(75%)/1 | | | |
| | | 29S6SvEvTac(25%)) | | | |
| B | 50500 | Wild Type | Ova(gV) + alum | Ova (gIII), | 8/8 |
| | | (C57BL/6NTac(75%)/1 | | 150 μg | |
| | | 29S6SvEvTac(25%)) | | | |
| C | 7467/7559 | Tslp ^{hu/hu}/Tslpr^{hu/hu} | saline + alum | saline | 7/7 |
| D | 7467/7559 | Tslp ^{hu/hu}/Tslpr^{hu/hu} | Ova(gV) + alum | Ova (gIII), | 8/8 |
| | | | | 150 μg | |
| E | 7267/7467/7559 | Tslp ^{hu/hu}/Tslpr ^{hu/hu}/ | saline + alum | saline | 8/8 |
| | | I17ra^{hu/hu} | | | |
| F | 7267/7467/7559 | Tslp ^{hu/hu}/Tslpr ^{hu/hu}/ | Ova(gV) + alum | Ova (gIII), | 7/7 |
| | | I17ra^{hu/hu} | | 150 μg | |

### CONCLUSIONS

Ova-alum model induced comparable levels of type 2 driven inflammation in double humanized (Tslp^{hu/hu}/Tslpr^{hu/hu}) and triple humanized (Tslp^{hu/hu}/Tslpr^{hu/hu}/Il7ra^{hu/hu}) mice, based on analysis of lung eosinophil infiltration, lung gene expression analysis and circulating antibody antibody levels.

1. A genetically modified rodent animal comprising in its genome:
a humanized Tslp gene comprising:
a rodent Tslp nucleic acid sequence, and
a human TSLP nucleic acid sequence,
wherein the humanized Tslp gene encodes a humanized Tslp polypeptide comprising a mature protein sequence having at least 95% identity to the mature protein sequence of a human TSLP protein, and wherein the rodent animal is a mouse or a rat.

2.-4. (canceled)

5. The genetically modified rodent animal of claim 1, wherein the humanized Tslp protein comprises a signal peptide having at least 95% identity to the signal peptide of a rodent Tslp protein.

6.-8. (canceled)

9. The genetically modified rodent animal of claim 1, wherein the human TSLP nucleic acid sequence comprises exon 1 from the codon for the first amino acid of the mature protein sequence, through the STOP codon in exon 4, of a human TSLP gene.

10.-13. (canceled)

14. The genetically modified rodent animal of claim 1, wherein the rodent animal is a mouse, and the humanized Tslp gene comprises (i) exon 1, and a 5′ portion of exon 2 coding for signal peptide amino acids, of a mouse Tslp gene, and (ii) exon 1 from the codon for the first amino acid of the mature protein sequence, through the STOP codon in exon 4, of a human TSLP gene.

15. The genetically modified rodent animal of claim 14, wherein the humanized Tslp gene further comprises the 3′ UTR of the mouse Tslp gene.

16. The genetically modified rodent animal of claim 1, wherein the humanized Tslp gene is operably linked to a rodent Tslp promoter.

17. The genetically modified rodent animal of claim 16, wherein the rodent Tslp promoter is the endogenous rodent Tslp promoter.

18. The genetically modified rodent animal of claim 1, wherein the humanized Tslp gene is located at an endogenous rodent Tslp locus.

19. The genetically modified rodent animal of claim 18, wherein the humanized Tslp gene is formed as a result of replacement of a rodent Tslp genomic DNA at an endogenous rodent Tslp locus with the human TSLP nucleic acid.

20.-22. (canceled)

23. The genetically modified rodent animal of claim 1, wherein the rodent is homozygous for the humanized Tslp gene.

24. The genetically modified rodent animal of claim 1, wherein the rodent is heterozygous for the humanized Tslp gene.

25. (canceled)

26. The genetically modified rodent animal of claim 1, whose genome further comprises a humanized Tslpr gene at an endogenous rodent Tslpr locus, a humanized Il7ra gene at an endogenous rodent Il7ra locus, or a combination thereof.

27. (canceled)

28. An isolated rodent tissue or cell, whose genome comprises a humanized Tslp gene comprising a rodent Tslp nucleic acid sequence and a human TSLP nucleic acid sequence, wherein the humanized Tslp gene encodes a humanized Tslp polypeptide comprising a mature protein sequence having at least 95% identity to a mature protein sequence of a human TSLP protein, wherein the rodent is a mouse or a rat.

29. (canceled)

30. The isolated rodent tissue or cell of claim 28 or 29, wherein the rodent cell is a rodent embryonic stem cell.

31. (canceled)

32. A rodent embryo, comprising the rodent embryonic stem cell of claim 30.

33. A method of making a genetically modified rodent wherein the rodent is a mouse or a rat, comprising:
modifying a rodent genome to comprise a humanized Tslp gene, wherein the humanized Tslp gene comprises a rodent Tslp nucleic acid sequence and a human TSLP nucleic acid sequence, and encodes a humanized Tslp polypeptide comprising a mature protein sequence having at least 95% identity with the mature protein sequence of a human TSLP protein; and
making a rodent comprising the modified rodent genome.

34. (canceled)

35. The method of claim 33, wherein said modifying comprises
introducing a nucleic acid molecule comprising the human TSLP nucleic acid sequence into the genome of a rodent embryonic stem (ES) cell,
obtaining a rodent ES cell in which the human TSLP nucleic acid sequence has been integrated into an endogenous Tslp locus to replace a rodent Tslp genomic DNA thereby forming the humanized Tslp gene, and
generating a rodent animal from the obtained rodent ES cell.

36. The method of claim 35, wherein the nucleic acid molecule further comprises a 5′ homology arm and a 3′ homology arm flanking the human TSLP nucleic acid sequence, and wherein the 5′ and 3′ homology arms are homologous to nucleic acid sequences at the endogenous rodent locus flanking the rodent Tslp genomic DNA to be replaced.

37. The method of claim 35, wherein the humanized Tslp gene is operably linked to the endogenous rodent Tslp promoter at the endogenous rodent Tslp locus.

38. The method of claim 33, wherein the human TSLP nucleic acid sequence encodes at least a substantial portion of the mature protein sequence of the human TSLP protein.

39. (canceled)

40. A targeting nucleic acid construct, comprising
a human TSLP nucleic acid sequence to be integrated into a rodent Tslp gene at an endogenous rodent Tslp locus, flanked by a 5′ nucleotide sequence and a 3′ nucleotide sequence that are homologous to nucleotide sequences at the rodent Tslp locus,
wherein integration of the human TSLP nucleic acid sequence into the rodent Tslp gene results in a replacement of a rodent Tslp genomic DNA with the human TSLP nucleic acid sequence thereby forming a humanized Tslp gene,
wherein the human TSLP nucleic acid sequence encodes at least a substantial portion of the mature protein sequence of a human TSLP protein, and
wherein the rodent is a mouse or a rat.

41.-124. (canceled)

125. A genetically modified rodent of claim 1, further comprising a humanized Sirpα gene, wherein the rodent is homozygous for RAG2−/− and IL2RG−/−.

126. A genetically modified rodent of claim 125, wherein the rodent further comprises a humanized Tpo gene, and/or a humanized GM-CSF/IL-3 locus.

127. (canceled)

128. A method of testing a candidate agent for treating an allergic condition, comprising
inducing an allergic condition in a genetically modified rodent animal as defined by claim 1;
administering a candidate agent to the genetically modified rodent animal; and
determining whether the candidate agent inhibits the allergic condition in the genetically modified rodent animal.

129. A method of testing a candidate agent for treating cancer, comprising
engrafting human cancer cells in a genetically modified rodent animal as defined by claim 1;
administering a candidate agent to the genetically modified rodent animal; and
determining whether the candidate agent inhibits the growth of the cancer cells in the genetically modified rodent animal.

130. The method of claim 128, wherein the candidate agent is a small molecule compound, a nucleic acid, or an antibody.

131.-136. (canceled)

137. The method of claim 129, wherein the candidate agent is a small molecule compound, a nucleic acid, or an antibody.
